SEQUENCE LISTING (59 sequences)

SEQ ID NO: 1 - protein, 444 aa, Homo sapiens
MVSQALRLLCLLLGLQGCLAAVFVTQEEAHGVLHRRRRANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP

SEQ ID NO: 2 - protein, 406 aa, Homo sapiens [sequence omitted]

SEQ ID NO: 3 - DNA, 2072 bp, Artificial Sequence
  Homo sapiens factor VII active site mutant immunoconjugate mRNA, complete CDS; NCBI Accession AF272774
aagctttgcagagatttcatcatggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggtgaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaagccccaagggcgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctcggggagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcgcgggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttcccggatccgcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataagcggccgc

SEQ ID NO: 4 - protein, 641 aa, Artificial Sequence
  Homo sapiens factor VII active site mutant immunoconjugate amino acid sequence
ANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCAGDSGGPHATHYRGTWYLTGIVSWGQGCATVGHFGVYTRVSQYIEWLQKLMRSEPRPGVLLRAPFPGSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 641 aa, Artificial Sequence
  Homo sapiens factor VII active site mutant immunoconjugate amino acid sequence, S344A and A341K (relative to SEQ ID NO4)
ANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDAGGPHATHYRGTWYLTGIVSWGQGCATVGHFGVYTRVSQYIEWLQKLMRSEPRPGVLLRAPFPGSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 2057 bp, Artificial Sequence
  FVII active site mutant coding sequence
ggatccaccatggtgtcacaagccctccgcctgctctgcctccttctcggacttcaaggatgtctggccgcagtgttcgtgactcaagaagaggcccacggggtgctgcaccggaggcgccgcgcgaacgcctttctggaggagctgcggccaggctcgctggaaagagagtgcaaagaggagcaatgctccttcgaggaggccagagaaattttcaaggacgccgaacggactaagctgttttggatctcatacagcgacggcgaccagtgtgccagcagcccgtgccagaacggcggttcgtgcaaggaccagctgcagtcctacatttgtttctgcctgccggcgttcgaaggaagaaactgtgaaacccacaaggatgaccagctgatctgcgtgaacgagaacggaggctgcgaacagtactgctccgaccacacggggactaagaggagctgcagatgccatgagggctatagcctgctggccgacggggtgtcctgcactccgactgtggaatacccgtgcggaaagatccccatcctggagaagcggaacgcatccaagccccaaggacgaattgtcggcggaaaggtctgccctaagggagagtgtccttggcaagtgctcctcttggtgaacggagcgcagctttgcggcggaactctgatcaacaccatctgggtggtgtcggcagctcactgctttgataagattaagaactggcggaacctgatcgcggtgcttggcgaacatgacctgtccgaacacgacggggacgagcagtcccggagagtggcccaggtcatcattccatccacctacgtgccggggaccacaaaccacgacatcgccttgctgcggctgcaccagccggtggtccttaccgatcacgtcgtgccgctctgcctgcccgagcgcacctttagcgagcgcaccctggccttcgtgcgcttctccctggtgtccggctggggacagctgctggacagaggcgccaccgccctggaactgatggtgctgaacgtgccacggctcatgacccaagattgtctgcagcagtcgcgcaaagtcggggattcaccgaacatcaccgagtacatgttctgcgccggttactccgatggatcgaaggactcgtgcgctggagactccggtggaccccatgctacccattatcgcggtacttggtacctgactggtattgtgtcctggggacagggctgcgccaccgtgggacatttcggcgtgtacacaagggtgtcacagtacatcgaatggttgcagaagctcatgcgcagcgaacctcgcccgggagtgctcctgagggcccctttccccggaagcgctgagcctaagagctgcgacaagacccacacttgtccgccatgccccgcgcccgaactgctgggtggcccatccgtgttcctgttcccgcctaagcccaaggacaccctgatgatttcgcggacccccgaagtgacttgcgtggtggtggacgtcagccacgaagatccggaagtcaagttcaattggtacgtggatggcgtcgaagtccataacgccaagaccaagccccgcgaggagcagtacaattccacttaccgggtggtgtccgtgttgaccgtgttgcaccaggactggctgaacggaaaggaatacaagtgcaaagtgtcgaataaggccctcccggccccgatcgagaaaacgatctccaaggccaagggccagcctcgggagccccaggtctacaccttgccgccgtcccgggatgagctgaccaagaaccaggtgtcactcacctgtctcgtcaaggggttctacccttccgacatcgccgtggaatgggagtcgaacgggcagccggaaaacaattacaagaccactcctcctgtcctggattccgacgggtcattcttcctgtactcaaagctgaccgtggacaagagcagatggcaacagggcaacgtgttcagctgctccgtgatgcacgaggccctgcacaaccactacacccagaagtccctgtccctctctcccggaaaatgagcggccgc

SEQ ID NO: 7 - DNA, 2057 bp, Artificial Sequence
  FVII active site mutant coding sequence
tctagaccaccatggtgtcacaagccctccgcctgctctgcctccttctcggacttcaaggatgtctggccgcagtgttcgtgactcaagaagaggcccacggggtgctgcaccggaggcgccgcgcgaacgcctttctggaggagctgcggccaggctcgctggaaagagagtgcaaagaggagcaatgctccttcgaggaggccagagaaattttcaaggacgccgaacggactaagctgttttggatctcatacagcgacggcgaccagtgtgccagcagcccgtgccagaacggcggttcgtgcaaggaccagctgcagtcctacatttgtttctgcctgccggcgttcgaaggaagaaactgtgaaacccacaaggatgaccagctgatctgcgtgaacgagaacggaggctgcgaacagtactgctccgaccacacggggactaagaggagctgcagatgccatgagggctatagcctgctggccgacggggtgtcctgcactccgactgtggaatacccgtgcggaaagatccccatcctggagaagcggaacgcatccaagccccaaggacgaattgtcggcggaaaggtctgccctaagggagagtgtccttggcaagtgctcctcttggtgaacggagcgcagctttgcggcggaactctgatcaacaccatctgggtggtgtcggcagctcactgctttgataagattaagaactggcggaacctgatcgcggtgcttggcgaacatgacctgtccgaacacgacggggacgagcagtcccggagagtggcccaggtcatcattccatccacctacgtgccggggaccacaaaccacgacatcgccttgctgcggctgcaccagccggtggtccttaccgatcacgtcgtgccgctctgcctgcccgagcgcacctttagcgagcgcaccctggccttcgtgcgcttctccctggtgtccggctggggacagctgctggacagaggcgccaccgccctggaactgatggtgctgaacgtgccacggctcatgacccaagattgtctgcagcagtcgcgcaaagtcggggattcaccgaacatcaccgagtacatgttctgcgccggttactccgatggatcgaaggactcgtgcgctggagactccggtggaccccatgctacccattatcgcggtacttggtacctgactggtattgtgtcctggggacagggctgcgccaccgtgggacatttcggcgtgtacacaagggtgtcacagtacatcgaatggttgcagaagctcatgcgcagcgaacctcgcccgggagtgctcctgagggcccctttccccggaagcgctgagcctaagagctgcgacaagacccacacttgtccgccatgccccgcgcccgaactgctgggtggcccatccgtgttcctgttcccgcctaagcccaaggacaccctgatgatttcgcggacccccgaagtgacttgcgtggtggtggacgtcagccacgaagatccggaagtcaagttcaattggtacgtggatggcgtcgaagtccataacgccaagaccaagccccgcgaggagcagtacaattccacttaccgggtggtgtccgtgttgaccgtgttgcaccaggactggctgaacggaaaggaatacaagtgcaaagtgtcgaataaggccctcccggccccgatcgagaaaacgatctccaaggccaagggccagcctcgggagccccaggtctacaccttgccgccgtcccgggatgagctgaccaagaaccaggtgtcactcacctgtctcgtcaaggggttctacccttccgacatcgccgtggaatgggagtcgaacgggcagccggaaaacaattacaagaccactcctcctgtcctggattccgacgggtcattcttcctgtactcaaagctgaccgtggacaagagcagatggcaacagggcaacgtgttcagctgctccgtgatgcacgaggccctgcacaaccactacacccagaagtccctgtccctctctcccggaaaatgaaccggt

SEQ ID NO: 8 - protein, 8 aa, Artificial Sequence
  Immunoconjugate linker - upper hinge region amino acid sequence
GSAEPKSC

SEQ ID NO: 9 - protein, 8 aa, Artificial Sequence
  Immunoconjugate mutant linker - upper hinge region amino acid sequence; C-terminal cysteine mutated to serine (relative to SEQ ID NO8)
GSAEPKSS

SEQ ID NO: 10 - protein, 8 aa, Artificial Sequence
  Immunoconjugate mutant linker - upper hinge region amino acid sequence; lysine at position 6 mutated to alanine (relative to SEQ ID NO9)
GSAEPASS

SEQ ID NO: 11 - protein, 4 aa, Artificial Sequence
  Immunoconjugate mutant linker - upper hinge region amino acid sequence; serine at position 2 mutated to glycine and residues 3-6 deleted (relative to SEQ ID NO9)
GGSS

SEQ ID NO: 12 - protein, 406 aa, Artificial Sequence
  Human factor VII active site mutant amino acid sequence
ANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCAGDSGGPHATHYRGTWYLTGIVSWGQGCATVGHFGVYTRVSQYIEWLQKLMRSEPRPGVLLRAPFP

SEQ ID NO: 13 - protein, 227 aa, Artificial Sequence
  IgG Fc amino acid sequence (with lower hinge)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 227 aa, Artificial Sequence
  IgG Fc amino acid sequence (with lower hinge) with protA mutation
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 15 - protein, 227 aa, Artificial Sequence
  IgG Fc amino acid sequence with T366Y knob mutation (relative to SEQ ID NO13)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 227 aa, Artificial Sequence
  IgG Fc amino acid sequence with T366Y knob mutation (relative to SEQ ID NO13) and protA mutation
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNRFTQKSLSLSPGK

SEQ ID NO: 17 - protein, 227 aa, Artificial Sequence
  IgG Fc amino acid sequence with Y407T hole mutation (relative to SEQ ID NO13)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 18 - protein, 227 aa, Artificial Sequence
  IgG Fc region with Y407T hole mutation (relative to SEQ ID NO13) and protA mutation
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNRFTQKSLSLSPGK

SEQ ID NO: 19 - protein, 5 aa, Artificial Sequence
  Upper hinge region amino acid sequence
EPKSC

SEQ ID NO: 20 - protein, 10 aa, Artificial Sequence
  Lower hinge region amino acid sequence
DKTHTCPPCP

SEQ ID NO: 21 - protein, 23 aa, Artificial Sequence
  IgG1 hinge amino acid sequence
EPKSCDKTHTCPPCPAPELLGGP

SEQ ID NO: 22 - protein, 18 aa, Artificial Sequence
  Truncated IgG1 hinge amino acid sequence
DKTHTCPPCPAPELLGGP

SEQ ID NO: 23 - protein, 23 aa, Artificial Sequence
  Mutated IgG1 hinge amino acid sequence - N-terminal cysteine mutated to serine (relative to SEQ ID NO21)
EPKSSDKTHTCPPCPAPELLGGP

SEQ ID NO: 24 - protein, 23 aa, Artificial Sequence
  Mutated IgG1 hinge amino acid sequence - N-terminal lysine mutated to alanine (relative to SEQ ID NO23)
EPASSDKTHTCPPCPAPELLGGP

SEQ ID NO: 25 - protein, 20 aa, Artificial Sequence
  IgG4 hinge amino acid sequence
ESKYGPPCPSCPAPEFLGGP

SEQ ID NO: 26 - protein, 232 aa, Artificial Sequence
  IgG1 Fc amino acid sequence comprising IgG1 hinge amino acid sequence
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 209 aa, Artificial Sequence
  IgG1 Fc amino acid sequence without hinge sequence
SVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 209 aa, Artificial Sequence
  IgG1 Fc amino acid sequence with protA mutation and without hinge sequence
SVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 29 - protein, 209 aa, Artificial Sequence
  IgG1 Fc amino acid sequence with T366Y knob mutation (relative to SEQ ID NO13) and without hinge sequence
SVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 209 aa, Artificial Sequence
  IgG1 Fc amino acid sequence with T366Y knob mutation (relative to SEQ ID NO13) with protA mutation and without hinge sequence
SVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNRFTQKSLSLSPGK

SEQ ID NO: 31 - protein, 209 aa, Artificial Sequence
  IgG1 Fc amino acid sequence with Y407T hole mutation (relative to SEQ ID NO13) and without hinge sequence
SVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 32 - protein, 209 aa, Artificial Sequence
  IgG1 Fc amino acid sequence with Y407T hole mutation (relative to SEQ ID NO13) with protA mutation and without hinge sequence
SVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNRFTQKSLSLSPGK

SEQ ID NO: 33 - protein, 406 aa, Artificial Sequence; identical to SEQ ID NO: 12
  Human factor VII active site mutant amino acid sequence

SEQ ID NO: 34 - protein, 406 aa, Artificial Sequence
  Human factor VII active site mutant amino acid sequence with mutations S344A and A341K relative to SEQ ID NO33
ANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDAGGPHATHYRGTWYLTGIVSWGQGCATVGHFGVYTRVSQYIEWLQKLMRSEPRPGVLLRAPFP

SEQ ID NO: 35 - DNA, 7304 bp, Artificial Sequence
  Nucleic acid sequence of pbudcd4.1 ICON Fc/knob and Fc/hole (construct JT075)
gcgcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttaccatggagtggtcctgggtgtttctgttcttcctgagcgtgaccaccggagtgcacagcgacaagacccacacctgtcccccttgtcctgcccccgaactgctgggaggcccttccgtgttcctgtttcctcccaagcccaaggacaccctgatgatcagcaggacccccgaggtgacctgtgtggtcgtggacgtgtcccacgaggaccctgaggtgaagttcaactggtacgtggacggcgtggaggtgcacaacgccaagacaaagccccgtgaggagcagtacaacagcacctacagggtggtgtccgtgctgacagtgctgcaccaggactggctgaatggcaaggagtacaagtgcaaagtgagcaacaaggccctgcccgcccccattgagaagaccatcagcaaggccaagggccagcccagagagcctcaggtgtacacactgcccccctccagggaggagatgaccaagaaccaggtgagcctgacctgtctggtgaagggcttctaccctagcgacatcgccgtggagtgggaaagcaacggccagcccgagaacaactacaagacaaccccccctgtgctggacagcgatggctccttcttcctgacaagcaagctgacagtggacaagagcaggtggcagcagggcaacgtcttctcctgcagcgtgatgcacgaagccctgcacaaccactacacccagaagagcctgagcctgagccccggctgatgatctagaggatccgaacaaaaactcatctcagaagaggatctgaatatgcataccggtcatcatcaccatcaccattgagtttgatccccgggaattcagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtttagttcctcaccttgtcgtattatactatgccgatatactatgccgatgattaattgtcaacacgtgctgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctcgtcgagctagcttcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctcgttggaatttgccctttttgagtttggatcttggttcattctcaagcctcagacagtggttcaaagtttttttcttccatttcaggtgtcgtgaacacgtggtcgcggccgcttcgaaggtaccaccatggtcagccaggccctcaggctgctctgtctcctgctgggcctccagggctgtctggctgccgtcttcgtgacccaggaggaggctcacggcgtgctccacaggaggagaagagctaacgctttcctcgaagagctgaggcccggcagcctggaaagggagtgcaaagaggagcagtgcagcttcgaagaagccagggaaattttcaaggacgccgagaggaccaagctgttctggatctcctatagcgacggcgatcagtgcgcttccagcccctgccagaacggaggctcctgcaaggatcagctgcagtcctacatttgcttctgtctgcccgcctttgagggcagaaactgtgagacccacaaggatgaccagctgatttgtgtgaatgagaacggcggatgcgaacagtactgctccgaccataccggaacaaagaggtcctgcagatgccatgagggctacagcctgctggccgatggagtgagctgcacccccaccgtggaatacccctgcggcaaaatccccatcctggagaagaggaatgccagcaaaccccagggcaggattgtgggcggaaaggtgtgccccaaaggagagtgtccttggcaggtgctgctgctcgtgaacggagcccagctgtgcggcggcacactcatcaataccatctgggtggtcagcgccgcccactgctttgacaagatcaagaactggaggaacctcatcgccgtgctgggagagcacgatctgagcgagcatgacggcgacgaacagtccaggagagtggcccaggtgatcatccctagcacatatgtccccggcaccaccaaccatgatatcgccctcctcaggctgcatcagcccgtggtgctgacagaccacgtggtgcctctctgcctgcccgagaggaccttcagcgagaggaccctggccttcgtgagattcagcctggtgagcggatggggccagctcctggatagaggcgccacagccctggagctgatggtgctgaacgtgcctagactgatgacccaagattgcctccagcagagcaggaaggtgggagactcccccaatatcacagagtatatgttttgcgccggctacagcgatggttctaaagattcctgtgctggcgacagcggaggcccccacgctacccactacagaggcacatggtatttaaccggaatcgtgtcctggggacaaggctgcgccaccgtgggccatttcggcgtgtacaccagggtcagccagtacatcgaatggctgcagaaactcatgaggtccgagcctagacccggcgtcctgctgagagcccctttccctgataaaacacacacctgtcccccttgtcctgctcctgagctcctgggcggaccttccgtgttcctgtttcctcccaaacccaaggacaccctgatgatctccaggacccccgaggtgacatgcgtcgtggtggacgtgagccacgaggaccccgaggtcaagttcaactggtacgtcgatggcgtggaggtccacaatgccaagaccaagcccagggaggagcaatataactccacctacagggtggtcagcgtgctcacagtgctgcaccaggattggctgaacggcaaagagtacaagtgcaaggtcagcaacaaagccctgcctgcccccatcgagaaaaccatctccaaggctaagggccagcccagggagcctcaggtgtacacactgcctcctagcagagaggagatgaccaagaaccaggtcagcctctactgcctggtcaaaggcttctacccctccgatattgctgtggaatgggagtccaacggccaacccgagaacaactacaagaccacaccccctgtgctggacagcgacggttcttttttcctgtacagcaagctgaccgtcgataagagcagatggcagcagggaaacgtgttcagctgttccgtcatgcacgaggccctgcacaaccactatacccagaaaagcctctccctgagccctggctgatgaagatctggccggctgggcccgtttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggcgccattcgccattcaggctgcgcaactgttgggaagggcgatcggtgcgggcctcttcgctattacgcca

SEQ ID NO: 36 - DNA, 3782 bp, Artificial Sequence
  5′ Vector of the construct of SEQ ID NO35
gcgcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttaccatggagtggtcctgggtgtttctgttcttcctgagcgtgaccaccggagtgcacagcgacaagacccacacctgtcccccttgtcctgcccccgaactgctgggaggcccttccgtgttcctgtttcctcccaagcccaaggacaccctgatgatcagcaggacccccgaggtgacctgtgtggtcgtggacgtgtcccacgaggaccctgaggtgaagttcaactggtacgtggacggcgtggaggtgcacaacgccaagacaaagccccgtgaggagcagtacaacagcacctacagggtggtgtccgtgctgacagtgctgcaccaggactggctgaatggcaaggagtacaagtgcaaagtgagcaacaaggccctgcccgcccccattgagaagaccatcagcaaggccaagggccagcccagagagcctcaggtgtacacactgcccccctccagggaggagatgaccaagaaccaggtgagcctgacctgtctggtgaagggcttctaccctagcgacatcgccgtggagtgggaaagcaacggccagcccgagaacaactacaagacaaccccccctgtgctggacagcgatggctccttcttcctgacaagcaagctgacagtggacaagagcaggtggcagcagggcaacgtcttctcctgcagcgtgatgcacgaagccctgcacaaccactacacccagaagagcctgagcctgagccccggctgatgatctagaggatccgaacaaaaactcatctcagaagaggatctgaatatgcataccggtcatcatcaccatcaccattgagtttgatccccgggaattcagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtttagttcctcaccttgtcgtattatactatgccgatatactatgccgatgattaattgtcaacacgtgctgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctcgtcgagctagcttcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctcgttggaatttgccctttttgagtttggatcttggttcattctcaagcctcagacagtggttcaaagtttttttcttccatttcaggtgtcgtgaacacgtggtcgcggccgcttcgaa

SEQ ID NO: 37 - DNA, 1491 bp, Artificial Sequence
  3′ Vector of the construct of SEQ ID NO35
ggccggctgggcccgtttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggcgccattcgccattcaggctgcgcaactgttgggaagggcgatcggtgcgggcctcttcgctattacgcca

SEQ ID NO: 38 - DNA, 2031 bp, Artificial Sequence
  Insert of the construct of SEQ ID NO35
ggtaccaccatggtcagccaggccctcaggctgctctgtctcctgctgggcctccagggctgtctggctgccgtcttcgtgacccaggaggaggctcacggcgtgctccacaggaggagaagagctaacgctttcctcgaagagctgaggcccggcagcctggaaagggagtgcaaagaggagcagtgcagcttcgaagaagccagggaaattttcaaggacgccgagaggaccaagctgttctggatctcctatagcgacggcgatcagtgcgcttccagcccctgccagaacggaggctcctgcaaggatcagctgcagtcctacatttgcttctgtctgcccgcctttgagggcagaaactgtgagacccacaaggatgaccagctgatttgtgtgaatgagaacggcggatgcgaacagtactgctccgaccataccggaacaaagaggtcctgcagatgccatgagggctacagcctgctggccgatggagtgagctgcacccccaccgtggaatacccctgcggcaaaatccccatcctggagaagaggaatgccagcaaaccccagggcaggattgtgggcggaaaggtgtgccccaaaggagagtgtccttggcaggtgctgctgctcgtgaacggagcccagctgtgcggcggcacactcatcaataccatctgggtggtcagcgccgcccactgctttgacaagatcaagaactggaggaacctcatcgccgtgctgggagagcacgatctgagcgagcatgacggcgacgaacagtccaggagagtggcccaggtgatcatccctagcacatatgtccccggcaccaccaaccatgatatcgccctcctcaggctgcatcagcccgtggtgctgacagaccacgtggtgcctctctgcctgcccgagaggaccttcagcgagaggaccctggccttcgtgagattcagcctggtgagcggatggggccagctcctggatagaggcgccacagccctggagctgatggtgctgaacgtgcctagactgatgacccaagattgcctccagcagagcaggaaggtgggagactcccccaatatcacagagtatatgttttgcgccggctacagcgatggttctaaagattcctgtgctggcgacagcggaggcccccacgctacccactacagaggcacatggtatttaaccggaatcgtgtcctggggacaaggctgcgccaccgtgggccatttcggcgtgtacaccagggtcagccagtacatcgaatggctgcagaaactcatgaggtccgagcctagacccggcgtcctgctgagagcccctttccctgataaaacacacacctgtcccccttgtcctgctcctgagctcctgggcggaccttccgtgttcctgtttcctcccaaacccaaggacaccctgatgatctccaggacccccgaggtgacatgcgtcgtggtggacgtgagccacgaggaccccgaggtcaagttcaactggtacgtcgatggcgtggaggtccacaatgccaagaccaagcccagggaggagcaatataactccacctacagggtggtcagcgtgctcacagtgctgcaccaggattggctgaacggcaaagagtacaagtgcaaggtcagcaacaaagccctgcctgcccccatcgagaaaaccatctccaaggctaagggccagcccagggagcctcaggtgtacacactgcctcctagcagagaggagatgaccaagaaccaggtcagcctctactgcctggtcaaaggcttctacccctccgatattgctgtggaatgggagtccaacggccaacccgagaacaactacaagaccacaccccctgtgctggacagcgacggttcttttttcctgtacagcaagctgaccgtcgataagagcagatggcagcagggaaacgtgttcagctgttccgtcatgcacgaggccctgcacaaccactatacccagaaaagcctctccctgagccctggctgatgaagatct

SEQ ID NO: 39 - protein, 670 aa, Artificial Sequence
  Translated insert of SEQ ID NO38
MVSQALRLLCLLLGLQGCLAAVFVTQEEAHGVLHRRRRANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCAGDSGGPHATHYRGTWYLTGIVSWGQGCATVGHFGVYTRVSQYIEWLQKLMRSEPRPGVLLRAPFPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - DNA, 8902 bp, Artificial Sequence
  Nucleic acid sequence of pVITRO2 ICON Fc/knob and Fc/hole (construct JT077)
cctgcaggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgatgatgcggttttggcagtacatcaatgggcgtggatagcggtttgactcacggggatttccaagtctccaccccattgacgtcaatgggagtttgttttgactagtcagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctaggcctccccgagcgccctgcctccgagggccggcgcaccataaaagaagccgccctagccacgtcccctcgcagttcggcggtcccgcgggtctgtctcaagcttgccgccagaacacaggtaagtgccgtgtgtggttcccgcgggcctggcctctttacgggttatggcccttgcgtgccttgaattacttccatgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgctaatctggtggcaccttcgcgcctgtctcgctgctttcgctaagtctctagccatttaaaatttttgataaccagctgcgacgctttttttctggcgagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaaactggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgcccgggagagcgggcgggtgagtcacccacacaaaggaaaagggcctttccttcctcatccgtcgcttcatgtgactccacggagtaccgggcgccgtccaggcacctcgattagttgtcgagcttttggagtacgtcgtctttaggttggggggaggggttttatgcgatggagtttccccacactgagtgggtggagactgaagagttaggccagcttggcacttgatgtaattctccttggaatttgccctttttgagtttggatcttgcctcattctcaagcctcagacagtggttcaaagtttttttcttccatttcaggtgtcgtgaaaactacccctaaaagccaccggcgatcgatgcggccgcaagcttaccatggagtggtcctgggtgtttctgttcttcctgagcgtgaccaccggagtgcacagcgacaagacccacacctgtcccccttgtcctgcccccgaactgctgggaggcccttccgtgttcctgtttcctcccaagcccaaggacaccctgatgatcagcaggacccccgaggtgacctgtgtggtcgtggacgtgtcccacgaggaccctgaggtgaagttcaactggtacgtggacggcgtggaggtgcacaacgccaagacaaagccccgtgaggagcagtacaacagcacctacagggtggtgtccgtgctgacagtgctgcaccaggactggctgaatggcaaggagtacaagtgcaaagtgagcaacaaggccctgcccgcccccattgagaagaccatcagcaaggccaagggccagcccagagagcctcaggtgtacacactgcccccctccagggaggagatgaccaagaaccaggtgagcctgacctgtctggtgaagggcttctaccctagcgacatcgccgtggagtgggaaagcaacggccagcccgagaacaactacaagacaaccccccctgtgctggacagcgatggctccttcttcctgacaagcaagctgacagtggacaagagcaggtggcagcagggcaacgtcttctcctgcagcgtgatgcacgaagccctgcacaaccactacacccagaagagcctgagcctgagccccggctgatgatctagactcgaggctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaatgttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttaattaacctgcagggcctgaaataacctctgaaagaggaacttggttaggtaccttctgaggctgaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagttccgccagagcgcgcgagggcctccagcggccgcccctcccccacagcaggggcggggtcccgcgcccaccggaaggagcgggctcggggcgggcggcgctgattggccggggcgggcctgacgccgacgcggctataagagaccacaagcgacccgcagggccagacgttcttcgccgaagcttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaagcaaccggtgtcgacggtaccaccatggtcagccaggccctcaggctgctctgtctcctgctgggcctccagggctgtctggctgccgtcttcgtgacccaggaggaggctcacggcgtgctccacaggaggagaagagctaacgctttcctcgaagagctgaggcccggcagcctggaaagggagtgcaaagaggagcagtgcagcttcgaagaagccagggaaattttcaaggacgccgagaggaccaagctgttctggatctcctatagcgacggcgatcagtgcgcttccagcccctgccagaacggaggctcctgcaaggatcagctgcagtcctacatttgcttctgtctgcccgcctttgagggcagaaactgtgagacccacaaggatgaccagctgatttgtgtgaatgagaacggcggatgcgaacagtactgctccgaccataccggaacaaagaggtcctgcagatgccatgagggctacagcctgctggccgatggagtgagctgcacccccaccgtggaatacccctgcggcaaaatccccatcctggagaagaggaatgccagcaaaccccagggcaggattgtgggcggaaaggtgtgccccaaaggagagtgtccttggcaggtgctgctgctcgtgaacggagcccagctgtgcggcggcacactcatcaataccatctgggtggtcagcgccgcccactgctttgacaagatcaagaactggaggaacctcatcgccgtgctgggagagcacgatctgagcgagcatgacggcgacgaacagtccaggagagtggcccaggtgatcatccctagcacatatgtccccggcaccaccaaccatgatatcgccctcctcaggctgcatcagcccgtggtgctgacagaccacgtggtgcctctctgcctgcccgagaggaccttcagcgagaggaccctggccttcgtgagattcagcctggtgagcggatggggccagctcctggatagaggcgccacagccctggagctgatggtgctgaacgtgcctagactgatgacccaagattgcctccagcagagcaggaaggtgggagactcccccaatatcacagagtatatgttttgcgccggctacagcgatggttctaaagattcctgtgctggcgacagcggaggcccccacgctacccactacagaggcacatggtatttaaccggaatcgtgtcctggggacaaggctgcgccaccgtgggccatttcggcgtgtacaccagggtcagccagtacatcgaatggctgcagaaactcatgaggtccgagcctagacccggcgtcctgctgagagcccctttccctgataaaacacacacctgtcccccttgtcctgctcctgagctcctgggcggaccttccgtgttcctgtttcctcccaaacccaaggacaccctgatgatctccaggacccccgaggtgacatgcgtcgtggtggacgtgagccacgaggaccccgaggtcaagttcaactggtacgtcgatggcgtggaggtccacaatgccaagaccaagcccagggaggagcaatataactccacctacagggtggtcagcgtgctcacagtgctgcaccaggattggctgaacggcaaagagtacaagtgcaaggtcagcaacaaagccctgcctgcccccatcgagaaaaccatctccaaggctaagggccagcccagggagcctcaggtgtacacactgcctcctagcagagaggagatgaccaagaaccaggtcagcctctactgcctggtcaaaggcttctacccctccgatattgctgtggaatgggagtccaacggccaacccgagaacaactacaagaccacaccccctgtgctggacagcgacggttcttttttcctgtacagcaagctgaccgtcgataagagcagatggcagcagggaaacgtgttcagctgttccgtcatgcacgaggccctgcacaaccactatacccagaaaagcctctccctgagccctggctgatgaagatctggatccacgcgtatcgattgtcgaccctaggagcaggtttccccaatgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccctatgaatacaactgactgtttgacaattaatcatcggcatagtatatcggcatagtataatacgactcactataggagggccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgaattcgctaggattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggttgctttgattacaacactggagagaaatgcagcatgttgctgattgcctgtcactaaaacaggccaaaaactgagtccttgggttgcatagaaagctg

SEQ ID NO: 41 - DNA, 4869 bp, Artificial Sequence
  5′ Vector of the construct of SEQ ID NO40
cctgcaggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgatgatgcggttttggcagtacatcaatgggcgtggatagcggtttgactcacggggatttccaagtctccaccccattgacgtcaatgggagtttgttttgactagtcagggccccaacccccccaagcccccatttcacaacacgctggcgctacaggcgcgtgacttccccttgctttggggcggggggctgagactcctatgtgctccggattggtcaggcacggccttcggccccgcctcctgccaccgcagattggccgctaggcctccccgagcgccctgcctccgagggccggcgcaccataaaagaagccgccctagccacgtcccctcgcagttcggcggtcccgcgggtctgtctcaagcttgccgccagaacacaggtaagtgccgtgtgtggttcccgcgggcctggcctctttacgggttatggcccttgcgtgccttgaattacttccatgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgctaatctggtggcaccttcgcgcctgtctcgctgctttcgctaagtctctagccatttaaaatttttgataaccagctgcgacgctttttttctggcgagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaaactggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgcccgggagagcgggcgggtgagtcacccacacaaaggaaaagggcctttccttcctcatccgtcgcttcatgtgactccacggagtaccgggcgccgtccaggcacctcgattagttgtcgagcttttggagtacgtcgtctttaggttggggggaggggttttatgcgatggagtttccccacactgagtgggtggagactgaagagttaggccagcttggcacttgatgtaattctccttggaatttgccctttttgagtttggatcttgcctcattctcaagcctcagacagtggttcaaagtttttttcttccatttcaggtgtcgtgaaaactacccctaaaagccaccggcgatcgatgcggccgcaagcttaccatggagtggtcctgggtgtttctgttcttcctgagcgtgaccaccggagtgcacagcgacaagacccacacctgtcccccttgtcctgcccccgaactgctgggaggcccttccgtgttcctgtttcctcccaagcccaaggacaccctgatgatcagcaggacccccgaggtgacctgtgtggtcgtggacgtgtcccacgaggaccctgaggtgaagttcaactggtacgtggacggcgtggaggtgcacaacgccaagacaaagccccgtgaggagcagtacaacagcacctacagggtggtgtccgtgctgacagtgctgcaccaggactggctgaatggcaaggagtacaagtgcaaagtgagcaacaaggccctgcccgcccccattgagaagaccatcagcaaggccaagggccagcccagagagcctcaggtgtacacactgcccccctccagggaggagatgaccaagaaccaggtgagcctgacctgtctggtgaagggcttctaccctagcgacatcgccgtggagtgggaaagcaacggccagcccgagaacaactacaagacaaccccccctgtgctggacagcgatggctccttcttcctgacaagcaagctgacagtggacaagagcaggtggcagcagggcaacgtcttctcctgcagcgtgatgcacgaagccctgcacaaccactacacccagaagagcctgagcctgagccccggctgatgatctagactcgaggctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaatgttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttaattaacctgcagggcctgaaataacctctgaaagaggaacttggttaggtaccttctgaggctgaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagttccgccagagcgcgcgagggcctccagcggccgcccctcccccacagcaggggcggggtcccgcgcccaccggaaggagcgggctcggggcgggcggcgctgattggccggggcgggcctgacgccgacgcggctataagagaccacaagcgacccgcagggccagacgttcttcgccgaagcttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaagca

SEQ ID NO: 42 - DNA, 1984 bp, Artificial Sequence
  3′ Vector of the construct of SEQ ID NO40
acgcgtatcgattgtcgaccctaggagcaggtttccccaatgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccctatgaatacaactgactgtttgacaattaatcatcggcatagtatatcggcatagtataatacgactcactataggagggccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgaattcgctaggattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggttgctttgattacaacactggagagaaatgcagcatgttgctgattgcctgtcactaaaacaggccaaaaactgagtccttgggttgcatagaaagctg

SEQ ID NO: 43 - DNA, 2049 bp, Artificial Sequence
  Insert of the construct of SEQ ID NO40
accggtgtcgacggtaccaccatggtcagccaggccctcaggctgctctgtctcctgctgggcctccagggctgtctggctgccgtcttcgtgacccaggaggaggctcacggcgtgctccacaggaggagaagagctaacgctttcctcgaagagctgaggcccggcagcctggaaagggagtgcaaagaggagcagtgcagcttcgaagaagccagggaaattttcaaggacgccgagaggaccaagctgttctggatctcctatagcgacggcgatcagtgcgcttccagcccctgccagaacggaggctcctgcaaggatcagctgcagtcctacatttgcttctgtctgcccgcctttgagggcagaaactgtgagacccacaaggatgaccagctgatttgtgtgaatgagaacggcggatgcgaacagtactgctccgaccataccggaacaaagaggtcctgcagatgccatgagggctacagcctgctggccgatggagtgagctgcacccccaccgtggaatacccctgcggcaaaatccccatcctggagaagaggaatgccagcaaaccccagggcaggattgtgggcggaaaggtgtgccccaaaggagagtgtccttggcaggtgctgctgctcgtgaacggagcccagctgtgcggcggcacactcatcaataccatctgggtggtcagcgccgcccactgctttgacaagatcaagaactggaggaacctcatcgccgtgctgggagagcacgatctgagcgagcatgacggcgacgaacagtccaggagagtggcccaggtgatcatccctagcacatatgtccccggcaccaccaaccatgatatcgccctcctcaggctgcatcagcccgtggtgctgacagaccacgtggtgcctctctgcctgcccgagaggaccttcagcgagaggaccctggccttcgtgagattcagcctggtgagcggatggggccagctcctggatagaggcgccacagccctggagctgatggtgctgaacgtgcctagactgatgacccaagattgcctccagcagagcaggaaggtgggagactcccccaatatcacagagtatatgttttgcgccggctacagcgatggttctaaagattcctgtgctggcgacagcggaggcccccacgctacccactacagaggcacatggtatttaaccggaatcgtgtcctggggacaaggctgcgccaccgtgggccatttcggcgtgtacaccagggtcagccagtacatcgaatggctgcagaaactcatgaggtccgagcctagacccggcgtcctgctgagagcccctttccctgataaaacacacacctgtcccccttgtcctgctcctgagctcctgggcggaccttccgtgttcctgtttcctcccaaacccaaggacaccctgatgatctccaggacccccgaggtgacatgcgtcgtggtggacgtgagccacgaggaccccgaggtcaagttcaactggtacgtcgatggcgtggaggtccacaatgccaagaccaagcccagggaggagcaatataactccacctacagggtggtcagcgtgctcacagtgctgcaccaggattggctgaacggcaaagagtacaagtgcaaggtcagcaacaaagccctgcctgcccccatcgagaaaaccatctccaaggctaagggccagcccagggagcctcaggtgtacacactgcctcctagcagagaggagatgaccaagaaccaggtcagcctctactgcctggtcaaaggcttctacccctccgatattgctgtggaatgggagtccaacggccaacccgagaacaactacaagaccacaccccctgtgctggacagcgacggttcttttttcctgtacagcaagctgaccgtcgataagagcagatggcagcagggaaacgtgttcagctgttccgtcatgcacgaggccctgcacaaccactatacccagaaaagcctctccctgagccctggctgatgaagatctggatcc

SEQ ID NO: 44 - protein, 670 aa, Artificial Sequence; identical to SEQ ID NO: 39
  Translated insert of SEQ ID NO43

SEQ ID NO: 45 - DNA, 4300 bp, Artificial Sequence
  Nucleic acid sequence of pSELECT-neo Fc/hole (construct JT090)
gcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgtgtcgacaccatggagtggagctgggtgtttctgttcttcctgagcgtgaccaccggcgttcattccgacaagacccacacatgtcccccctgccctgctcctgaactgctgggaggccccagcgtgttcctgtttcctcccaagcccaaggacaccctgatgatcagccggacccctgaagtgacctgcgtggtggtggacgtgtcccacgaggaccccgaggtgaagttcaattggtacgtggacggcgtggaagtccacaacgctaagaccaagccccgggaggaacagtacaacagcacctaccgggtggtgtccgtgctgaccgtgctgcaccaggactggctgaacggcaaggagtacaagtgcaaagtgtccaacaaggccctgcccgcccccatcgagaagaccatcagcaaggccaagggacagcctagggagcctcaggtctacaccctgcccccttccagggaggagatgaccaagaatcaggtgtccctgacctgcctggtgaagggcttctaccccagcgacatcgccgtggagtgggagagcaacggccagcctgagaacaactacaagaccacccctcccgtgctggatagcgacggctccttcttcctgaccagcaaactgaccgtggacaagagcaggtggcagcagggcaacgtgttctcctgctccgtgatgcacgaggctctgcacaaccggttcacccagaagagcctgtccctgagccctggatgatgagctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagctttagaagaactcatcaagaagtctgtagaaggcaattctctgggagtcaggggctgcaatgccatagagcactaggaacctgtctgcccactctccccctagctcttctgctatgtccctggttgctagggcaatgtcctggtacctgtcagccactcccagcctgccacagtctatgaagccagagaaccttccattttcaaccatgatgttgggaaggcaggcatccccatgagtcaccactaggtcctcaccatctggcatggatgccttgagcctggcaaatagttcagcaggggccaggccctggtgttcttcatccaagtcatcttggtccaccaggccagcctccatcctggttctggccctctctatcctgtgcttggcctggtggtcaaaggggcaggtggctgggtcaagggtgtggagtcttctcatggcatcagccatgattgacactttctcagctggagctaggtgagaggaaaggaggtcctgcccaggcacctcacctagtaggagccagtcccttccagcttctgtgaccacatcaaggacagctgcacaggggaccccagttgttgccaaccaggagagtctggcagcctcatcctggagctcattgagagccccactgaggtctgtctttacaaaaaggactggcctgccttgggctgaaagtctgaaaactgctgcatcagagcaaccaatggtctgctgtgcccagtcatagccaaacagtctctcaacccaggcagctggagaacctgcatgtaggccatcttgttcaatcatgatggccctcctatagtgagtcgtattatactatgccgatatactatgccgatgattaattgtcaaaacagcgtggatggcgtctccagcttatctgacggttcactaaacgagctctgcttatatagacctcccaccgtacacgcctaccgcccatttgcgtcaatggggcggagttgttacgacattttggaaagtcccgttgatttactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatca

SEQ ID NO: 46 - DNA, 725 bp, Artificial Sequence
  5′ Vector of the construct of SEQ ID NO45
gcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgt

SEQ ID NO: 47 - DNA, 2819 bp, Artificial Sequence
  3′ Vector of the construct of SEQ ID NO45
tggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagctttagaagaactcatcaagaagtctgtagaaggcaattctctgggagtcaggggctgcaatgccatagagcactaggaacctgtctgcccactctccccctagctcttctgctatgtccctggttgctagggcaatgtcctggtacctgtcagccactcccagcctgccacagtctatgaagccagagaaccttccattttcaaccatgatgttgggaaggcaggcatccccatgagtcaccactaggtcctcaccatctggcatggatgccttgagcctggcaaatagttcagcaggggccaggccctggtgttcttcatccaagtcatcttggtccaccaggccagcctccatcctggttctggccctctctatcctgtgcttggcctggtggtcaaaggggcaggtggctgggtcaagggtgtggagtcttctcatggcatcagccatgattgacactttctcagctggagctaggtgagaggaaaggaggtcctgcccaggcacctcacctagtaggagccagtcccttccagcttctgtgaccacatcaaggacagctgcacaggggaccccagttgttgccaaccaggagagtctggcagcctcatcctggagctcattgagagccccactgaggtctgtctttacaaaaaggactggcctgccttgggctgaaagtctgaaaactgctgcatcagagcaaccaatggtctgctgtgcccagtcatagccaaacagtctctcaacccaggcagctggagaacctgcatgtaggccatcttgttcaatcatgatggccctcctatagtgagtcgtattatactatgccgatatactatgccgatgattaattgtcaaaacagcgtggatggcgtctccagcttatctgacggttcactaaacgagctctgcttatatagacctcccaccgtacacgcctaccgcccatttgcgtcaatggggcggagttgttacgacattttggaaagtcccgttgatttactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatca

SEQ ID NO: 48 - DNA, 756 bp, Artificial Sequence
  Insert of the construct of SEQ ID NO45
gtcgacaccatggagtggagctgggtgtttctgttcttcctgagcgtgaccaccggcgttcattccgacaagacccacacatgtcccccctgccctgctcctgaactgctgggaggccccagcgtgttcctgtttcctcccaagcccaaggacaccctgatgatcagccggacccctgaagtgacctgcgtggtggtggacgtgtcccacgaggaccccgaggtgaagttcaattggtacgtggacggcgtggaagtccacaacgctaagaccaagccccgggaggaacagtacaacagcacctaccgggtggtgtccgtgctgaccgtgctgcaccaggactggctgaacggcaaggagtacaagtgcaaagtgtccaacaaggccctgcccgcccccatcgagaagaccatcagcaaggccaagggacagcctagggagcctcaggtctacaccctgcccccttccagggaggagatgaccaagaatcaggtgtccctgacctgcctggtgaagggcttctaccccagcgacatcgccgtggagtgggagagcaacggccagcctgagaacaactacaagaccacccctcccgtgctggatagcgacggctccttcttcctgaccagcaaactgaccgtggacaagagcaggtggcagcagggcaacgtgttctcctgctccgtgatgcacgaggctctgcacaaccggttcacccagaagagcctgtccctgagccctggatgatgagctagc

SEQ ID NO: 49 - protein, 245 aa, Artificial Sequence
  Translated insert of SEQ ID NO48
MEWSWVFLFFLSVTTGVHSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNRFTQKSLSLSPG

SEQ ID NO: 50 - DNA, 4110 bp, Artificial Sequence
  Nucleic acid sequence of pSELECT-puro Fc/hole (construct JT091)
gcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgtgtcgacaccatggagtggagctgggtgtttctgttcttcctgagcgtgaccaccggcgttcattccgacaagacccacacatgtcccccctgccctgctcctgaactgctgggaggccccagcgtgttcctgtttcctcccaagcccaaggacaccctgatgatcagccggacccctgaagtgacctgcgtggtggtggacgtgtcccacgaggaccccgaggtgaagttcaattggtacgtggacggcgtggaagtccacaacgctaagaccaagccccgggaggaacagtacaacagcacctaccgggtggtgtccgtgctgaccgtgctgcaccaggactggctgaacggcaaggagtacaagtgcaaagtgtccaacaaggccctgcccgcccccatcgagaagaccatcagcaaggccaagggacagcctagggagcctcaggtctacaccctgcccccttccagggaggagatgaccaagaatcaggtgtccctgacctgcctggtgaagggcttctaccccagcgacatcgccgtggagtgggagagcaacggccagcctgagaacaactacaagaccacccctcccgtgctggatagcgacggctccttcttcctgaccagcaaactgaccgtggacaagagcaggtggcagcagggcaacgtgttctcctgctccgtgatgcacgaggctctgcacaaccggttcacccagaagagcctgtccctgagccctggatgatgagctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagctcaggtttaagctccaggcttccttgtcatgcaccaagttcttgggccttctggaacctcaacatcagctgtcacagtgaatcccagtctttcataaaaaggcaggtttctgggagcagaagtttccagaaaggcaggaactccagccctttcagcagcttcaactccaggcagaacaacagcagatcccagaccctttccctggtggtcagggctcactccaacagttgccagaaaccaagctggctcttttggcctgtgtggtgccagcagaccttccatttgttgttgtgctgccagcctgcttccagagagctcagccattcttggtccaatttcagcaaaaacagcaccagcttcaacagactcaggtgttgtccaaactgcaacagcagctccatcatctgcaacccaaacttttccaatgtccagtcccactctggtgaggaagagttcttgcagttctgtcaccctctcaatgtgcctgtcagggtcaactgtgtgccttgttgcagggtagtctgcaaaagcagcagccagtgttctcacagctcttggaacatcatctctggttgccagcctcactgtgggtttgtactcagtcatggtggccctcctatagtgagtcgtattatactatgccgatatactatgccgatgattaattgtcaaaacagcgtggatggcgtctccagcttatctgacggttcactaaacgagctctgcttatatagacctcccaccgtacacgcctaccgcccatttgcgtcaatggggcggagttgttacgacattttggaaagtcccgttgatttactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatca

SEQ ID NO: 51 - DNA, 725 bp, Artificial Sequence; identical to SEQ ID NO: 46
  5′ Vector of the construct of SEQ ID NO50

SEQ ID NO: 52 - DNA, 2629 bp, Artificial Sequence
  3′ Vector of the construct of SEQ ID NO50
tggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagctcaggtttaagctccaggcttccttgtcatgcaccaagttcttgggccttctggaacctcaacatcagctgtcacagtgaatcccagtctttcataaaaaggcaggtttctgggagcagaagtttccagaaaggcaggaactccagccctttcagcagcttcaactccaggcagaacaacagcagatcccagaccctttccctggtggtcagggctcactccaacagttgccagaaaccaagctggctcttttggcctgtgtggtgccagcagaccttccatttgttgttgtgctgccagcctgcttccagagagctcagccattcttggtccaatttcagcaaaaacagcaccagcttcaacagactcaggtgttgtccaaactgcaacagcagctccatcatctgcaacccaaacttttccaatgtccagtcccactctggtgaggaagagttcttgcagttctgtcaccctctcaatgtgcctgtcagggtcaactgtgtgccttgttgcagggtagtctgcaaaagcagcagccagtgttctcacagctcttggaacatcatctctggttgccagcctcactgtgggtttgtactcagtcatggtggccctcctatagtgagtcgtattatactatgccgatatactatgccgatgattaattgtcaaaacagcgtggatggcgtctccagcttatctgacggttcactaaacgagctctgcttatatagacctcccaccgtacacgcctaccgcccatttgcgtcaatggggcggagttgttacgacattttggaaagtcccgttgatttactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatca

SEQ ID NO: 53 - DNA, 756 bp, Artificial Sequence; identical to SEQ ID NO: 48
  Insert of the construct of SEQ ID NO50

SEQ ID NO: 54 - protein, 245 aa, Artificial Sequence; identical to SEQ ID NO: 49
  Translated insert of SEQ ID NO53

SEQ ID NO: 55 - DNA, 5593 bp, Artificial Sequence
  Nucleic acid sequence of pSELECT-puro FVII-Fc/hole (construct JT092)
gcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgtgtcgacggtaccaccatggtcagccaggccctcaggctgctctgtctcctgctgggcctccagggctgtctggctgccgtcttcgtgacccaggaggaggctcacggcgtgctccacaggaggagaagagctaacgctttcctcgaagagctgaggcccggcagcctggaaagggagtgcaaagaggagcagtgcagcttcgaagaagccagggaaattttcaaggacgccgagaggaccaagctgttctggatctcctatagcgacggcgatcagtgcgcttccagcccctgccagaacggaggctcctgcaaggatcagctgcagtcctacatttgcttctgtctgcccgcctttgagggcagaaactgtgagacccacaaggatgaccagctgatttgtgtgaatgagaacggcggatgcgaacagtactgctccgaccataccggaacaaagaggtcctgcagatgccatgagggctacagcctgctggccgatggagtgagctgcacccccaccgtggaatacccctgcggcaaaatccccatcctggagaagaggaatgccagcaaaccccagggcaggattgtgggcggaaaggtgtgccccaaaggagagtgtccttggcaggtgctgctgctcgtgaacggagcccagctgtgcggcggcacactcatcaataccatctgggtggtcagcgccgcccactgctttgacaagatcaagaactggaggaacctcatcgccgtgctgggagagcacgatctgagcgagcatgacggcgacgaacagtccaggagagtggcccaggtgatcatccctagcacatatgtccccggcaccaccaaccatgatatcgccctcctcaggctgcatcagcccgtggtgctgacagaccacgtggtgcctctctgcctgcccgagaggaccttcagcgagaggaccctggccttcgtgagattcagcctggtgagcggatggggccagctcctggatagaggcgccacagccctggagctgatggtgctgaacgtgcctagactgatgacccaagattgcctccagcagagcaggaaggtgggagactcccccaatatcacagagtatatgttttgcgccggctacagcgatggttctaaagattcctgtgctggcgacagcggaggcccccacgctacccactacagaggcacatggtatttaaccggaatcgtgtcctggggacaaggctgcgccaccgtgggccatttcggcgtgtacaccagggtcagccagtacatcgaatggctgcagaaactcatgaggtccgagcctagacccggcgtcctgctgagagcccctttccctgataaaacacacacctgtcccccttgtcctgctcctgagctcctgggcggaccttccgtgttcctgtttcctcccaaacccaaggacaccctgatgatctccaggacccccgaggtgacatgcgtcgtggtggacgtgagccacgaggaccccgaggtcaagttcaactggtacgtcgatggcgtggaggtccacaatgccaagaccaagcccagggaggagcaatataactccacctacagggtggtcagcgtgctcacagtgctgcaccaggattggctgaacggcaaagagtacaagtgcaaggtcagcaacaaagccctgcctgcccccatcgagaaaaccatctccaaggctaagggccagcccagggagcctcaggtgtacacactgcctcctagcagagaggagatgaccaagaaccaggtcagcctctactgcctggtcaaaggcttctacccctccgatattgctgtggaatgggagtccaacggccaacccgagaacaactacaagaccacaccccctgtgctggacagcgacggttcttttttcctgtacagcaagctgaccgtcgataagagcagatggcagcagggaaacgtgttcagctgttccgtcatgcacgaggccctgcacaaccactatacccagaaaagcctctccctgagccctggctgatgaagatctggatccgctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagctttagaagaactcatcaagaagtctgtagaaggcaattctctgggagtcaggggctgcaatgccatagagcactaggaacctgtctgcccactctccccctagctcttctgctatgtccctggttgctagggcaatgtcctggtacctgtcagccactcccagcctgccacagtctatgaagccagagaaccttccattttcaaccatgatgttgggaaggcaggcatccccatgagtcaccactaggtcctcaccatctggcatggatgccttgagcctggcaaatagttcagcaggggccaggccctggtgttcttcatccaagtcatcttggtccaccaggccagcctccatcctggttctggccctctctatcctgtgcttggcctggtggtcaaaggggcaggtggctgggtcaagggtgtggagtcttctcatggcatcagccatgattgacactttctcagctggagctaggtgagaggaaaggaggtcctgcccaggcacctcacctagtaggagccagtcccttccagcttctgtgaccacatcaaggacagctgcacaggggaccccagttgttgccaaccaggagagtctggcagcctcatcctggagctcattgagagccccactgaggtctgtctttacaaaaaggactggcctgccttgggctgaaagtctgaaaactgctgcatcagagcaaccaatggtctgctgtgcccagtcatagccaaacagtctctcaacccaggcagctggagaacctgcatgtaggccatcttgttcaatcatgatggccctcctatagtgagtcgtattatactatgccgatatactatgccgatgattaattgtcaaaacagcgtggatggcgtctccagcttatctgacggttcactaaacgagctctgcttatatagacctcccaccgtacacgcctaccgcccatttgcgtcaatggggcggagttgttacgacattttggaaagtcccgttgatttactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatca

SEQ ID NO: 56 - DNA, 725 bp, Artificial Sequence; identical to SEQ ID NO: 46
  5′ Vector of the construct of SEQ ID NO55

SEQ ID NO: 57 - DNA, 2819 bp, Artificial Sequence; identical to SEQ ID NO: 47
  3′ Vector of the construct of SEQ ID NO55

SEQ ID NO: 58 - DNA, 2049 bp, Artificial Sequence
  Insert of the construct of SEQ ID NO55
gtcgacggtaccaccatggtcagccaggccctcaggctgctctgtctcctgctgggcctccagggctgtctggctgccgtcttcgtgacccaggaggaggctcacggcgtgctccacaggaggagaagagctaacgctttcctcgaagagctgaggcccggcagcctggaaagggagtgcaaagaggagcagtgcagcttcgaagaagccagggaaattttcaaggacgccgagaggaccaagctgttctggatctcctatagcgacggcgatcagtgcgcttccagcccctgccagaacggaggctcctgcaaggatcagctgcagtcctacatttgcttctgtctgcccgcctttgagggcagaaactgtgagacccacaaggatgaccagctgatttgtgtgaatgagaacggcggatgcgaacagtactgctccgaccataccggaacaaagaggtcctgcagatgccatgagggctacagcctgctggccgatggagtgagctgcacccccaccgtggaatacccctgcggcaaaatccccatcctggagaagaggaatgccagcaaaccccagggcaggattgtgggcggaaaggtgtgccccaaaggagagtgtccttggcaggtgctgctgctcgtgaacggagcccagctgtgcggcggcacactcatcaataccatctgggtggtcagcgccgcccactgctttgacaagatcaagaactggaggaacctcatcgccgtgctgggagagcacgatctgagcgagcatgacggcgacgaacagtccaggagagtggcccaggtgatcatccctagcacatatgtccccggcaccaccaaccatgatatcgccctcctcaggctgcatcagcccgtggtgctgacagaccacgtggtgcctctctgcctgcccgagaggaccttcagcgagaggaccctggccttcgtgagattcagcctggtgagcggatggggccagctcctggatagaggcgccacagccctggagctgatggtgctgaacgtgcctagactgatgacccaagattgcctccagcagagcaggaaggtgggagactcccccaatatcacagagtatatgttttgcgccggctacagcgatggttctaaagattcctgtgctggcgacagcggaggcccccacgctacccactacagaggcacatggtatttaaccggaatcgtgtcctggggacaaggctgcgccaccgtgggccatttcggcgtgtacaccagggtcagccagtacatcgaatggctgcagaaactcatgaggtccgagcctagacccggcgtcctgctgagagcccctttccctgataaaacacacacctgtcccccttgtcctgctcctgagctcctgggcggaccttccgtgttcctgtttcctcccaaacccaaggacaccctgatgatctccaggacccccgaggtgacatgcgtcgtggtggacgtgagccacgaggaccccgaggtcaagttcaactggtacgtcgatggcgtggaggtccacaatgccaagaccaagcccagggaggagcaatataactccacctacagggtggtcagcgtgctcacagtgctgcaccaggattggctgaacggcaaagagtacaagtgcaaggtcagcaacaaagccctgcctgcccccatcgagaaaaccatctccaaggctaagggccagcccagggagcctcaggtgtacacactgcctcctagcagagaggagatgaccaagaaccaggtcagcctctactgcctggtcaaaggcttctacccctccgatattgctgtggaatgggagtccaacggccaacccgagaacaactacaagaccacaccccctgtgctggacagcgacggttcttttttcctgtacagcaagctgaccgtcgataagagcagatggcagcagggaaacgtgttcagctgttccgtcatgcacgaggccctgcacaaccactatacccagaaaagcctctccctgagccctggctgatgaagatctggatccgctagc

SEQ ID NO: 59 - protein, 670 aa, Artificial Sequence; identical to SEQ ID NO: 39
  Translated insert of SEQ ID NO58

(19) United States
(12) Patent Application Publication
(10) Pub. No.: US 2019/0153119 A1
(43) Pub. Date: May 23, 2019

(54) COMPOSITIONS AND METHODS FOR TREATING DISORDERS ASSOCIATED WITH NEOVASCULARIZATION

(71) Applicant: Iconic Therapeutics, Inc., South San Francisco, CA (US)

(72) Inventors: Thi-Sau MIGONE, Burlingame, CA (US); Jan-Willem THEUNISSEN, Foster City, CA (US)

(21) Appl. No.: 16/093,837

(22) PCT Filed: Apr. 14, 2017
(86) PCT No.: PCT/US2017/027808

§ 371 Date: Oct. 15, 2018

Related U.S. Application Data
(60) Provisional application No. 62/322,540, filed on Apr. 14, 2016.

(51) Int. Cl. C07K 16/46; C07K 14/745; C12N 9/64; A61K 47/68; A61P 27/02; A61P 3/00; A61K 9/00

(52) U.S. Cl. CPC C07K 16/46; C07K 14/745; C12N 9/6437; A61K 47/68; A61K 38/00; A61P 3/00; A61K 9/0019; C12Y 304/21021; C07K 2319/30; A61P 27/02

(57) ABSTRACT

Provided herein are immunoconjugate fusion proteins for the treatment of disorders associated with neovascularization (e.g. tumor-associated neovascularization, e.g., ocular melanoma), and symptoms associated with the same. The methods comprise administering the patient in one or more dosing sessions, a composition comprising an effective amount of any one or more of the immunoconjugate proteins described herein, wherein each immunoconjugate comprises at least one mutated Factor VIIa (FVIIa) protein conjugated to an immunoglobulin Ig Fc dimer.

### CROSS REFERENCE TO RELATED APPLICATIONS

This application claims the benefit of priority to U.S. Provisional Application Ser. No. 62/322,540, filed on Apr. 14, 2016, and is herein incorporated by reference in its entirety for all purposes.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is ICTH_003_01WO_ST25.txt. The text file is 168 KB, was created on Apr. 13, 2017, and is being submitted electronically via EFS-Web.

### BACKGROUND OF THE INVENTION

Neovascularization (interchangeably referred to herein as angiogenesis) generally refers to the growth of existing blood vessels and the formation of new blood vessels, and is observed in a variety of diseases. Neovascularization can enable solid tumor growth and metastasis, cause visual malfunction in ocular disorders, promote leukocyte extravasation in inflammatory disorders, and/or influence the outcome of cardiovascular diseases such as atherosclerosis.

Neovascularization occurs only infrequently in healthy adults, primarily during wound healing and certain reproductive events. Neovascularization that promotes or causes disease can be referred to as pathological neovascularization and such neovasculature may be referred to as pathological neovasculature (PNV). PNV is intrinsic to several diseases and includes tumorigenic neovascularization that promotes the growth of solid cancers and melanomas, including ocular melanoma.

The survival and growth of a solid tumor depend critically on the development of a supporting neovasculature (Folkman, J. (1995) N. Engl. J. Med. 333, 1757-1763). Tumor-associated neovascularization is critical in supporting cancer progression as it supplies oxygen and nutrients. Neovascularization also plays a significant role in rheumatoid arthritis (Szekanez, Z., et al. (1998) J. Invest. Med. 46, 27-41). Neovascularization underlies the majority of eye diseases that result in catastrophic loss of vision (Friedlander, M., et al. (1996) Proc. Natl. Acad. Sci. USA 93, 9764-9769), for example in ocular melanoma and age-related macular degeneration (AMD).

Neovascularization is involved in ocular melanoma, which is a melanoma of the uveal tract, including the choroid, iris, and ciliary body. The standard of care for small- and medium-sized ocular tumors is typically radiation. Over 60% of patients receiving some form of radiation, either plaque radiotherapy (brachytherapy) or proton beam radiation. However, radiotherapies are highly invasive and can lead to complications such as retinopathy, cataracts, glaucoma, and significant vision loss. In the case of large tumors, surgical removal of the tumor or eye may be performed. None of the aforementioned treatments affect the rate at which metastatic disease occurs. While local recurrence in the eye is rare, nearly half of all uveal melanomas will develop distant metastasis, primarily in the liver.

Age-related macular degeneration (AMD) refers to the chronic, progressive degenerative pathology of the macula that results in loss of central vision. Neovascular AMD (also referred to as exudative or “wet” AMD) is the leading cause of severe vision loss and blindness in elderly patients over the age of 50 in the industrialized world.

Tissue factor (TF) is a cytokine receptor present on vascular endothelial cells. It is an integral membrane glycoprotein with an intracellular terminal domain, a transmembrane domain and an extracellular binding domain for Factor VII (FVII) and Factor VIIa (FVIIa activated Factor VII). TF acts as a cell-associated receptor for the activated form of coagulation Factor VII (FVIIa); the formation of this complex initiates blood coagulation and mediates cellular signaling. TF has been implicated in the process of neovascularization and the inflammatory cascade of cytokine release, both processes involved in PNV. In cancer, TF plays a significant role in multiple aspects of cancer growth in modulating tumor growth, tumor angiogenesis, pathogenic neovascularization, metastasis, and thrombosis. In the tumor microenvironment, relative to non-transformed cells, TF is over-expressed by tumor, vascular, stromal, and some inflammatory cells.

Factor VII (FVII) is a serine protease enzyme that causes the coagulation of blood in the coagulation cascade. During typical homeostasis the endothelium of the vasculature system separates TF from its circulating ligand, FVII. Breakage of the endothelial barrier leads to the exposure of extravascular TF to FVII, thus leading to the rapid activation of the coagulation cascade.

One approach to the treatment of disorders and diseases associated with pathological neovascularization, and particularly of cancer, has been to compromise the function or growth of the neovasculature. This may be achieved by inhibiting TF, but because TF exerts multiple biological activities crucial to homeostasis, simply inhibiting TF is not a viable pharmacological solution. It is known that knocking out the TF gene in mice is lethal, and the inhibition of TF can induce hemorrhaging (Chu. Int. J. Inflam. 2011. 2011:30).

Thus there is an unmet medical need for new therapeutic strategies for treating disorders associated with neovascularization (e.g. tumor-associated neovascularization) which are less invasive, provide a durable benefit, and slowing the onset or even prevent further disease progression (such as metastasis). The present invention addresses this and other needs.

Patents, patent applications, patent application publications, journal articles and protocols referenced herein are incorporated by reference in their entireties, for all purposes.

### SUMMARY OF THE INVENTION

In some embodiments, the disclosure is drawn to an immunoconjugate comprising two dimerized immunoglobulin (Ig) Fc monomers, and a mutated factor VII protein, wherein the mutated factor VII protein is fused to only one of the Fc monomers, and wherein the mutated factor VII protein exhibits a decreased coagulation response in a mammalian host, as compared to a wild-type factor VII protein. In further embodiments, the mutated factor VII protein exhibits no coagulation response in a mammalian host. In further embodiments, the immunoconjugate comprises a linker sequence between the Ig Fc monomer and the factor VII protein. In some embodiments, the Ig Fc monomers comprise a hinge sequence. In some embodiments, one or more of the Ig Fc monomers comprise a linker sequence and a hinge sequence. In some embodiments, the linker and/or hinge sequences comprise one or more cysteine amino acid residues. In some embodiments, the two dimerized Ig Fc monomers are linked together by one or more disulfide bonds. In some embodiments, the dimerized Ig Fc is a homodimer or a heterodimer. In some embodiments, the heterodimer comprises a Fc monomer with a T366Y mutation and a Fc monomer with a Y407T mutation or comprises a Fc monomer with a mutation corresponding to a T366Y mutation and a Fc monomer with a mutation corresponding to a Y407T mutation. In some embodiments, the one or more of the Ig Fc monomers consist of the amino acid sequence of SEQ ID NO:27.

In one embodiment, the presence of a linker results in an increase in the production yield of the immunoconjugate, as compared to the immunoconjugate lacking a linker. In one embodiment, the Ig Fc monomers are human IgG Fc monomers. In a further embodiment, the human IgG Fc monomers are selected from IgG, IgG2, IgG3, and IgG4. In one embodiment, the human IgG Fc monomers are those of IgG1. In one embodiment, the human IgG Fc monomers comprise the amino acid sequence selected from SEQ ID NO:13, 15, 17, 26, 27, 29, and 31. In one embodiment, the mutated human FVII protein comprises a single point mutation at Lys341 or Ser344. In one embodiment, the single point mutation is Lys341 to Ala341. In one embodiment, the single point mutation is Ser344 to Ala344. In one embodiment, the mutated human FVII protein comprises a point mutation at Lys341 and Ser344. In one embodiment, the mutated human FVII protein further comprises a Ser344 to Ala344 point mutation. In one embodiment, the mutated human FVII protein comprises the amino acid sequence of SEQ ID NO:12.

In one embodiment, the immunoconjugate is fucosylated, N-glycosylated, O-glycosylated, or afucosylated. In one embodiment, the hinge sequence comprises an amino acid sequence that shares at least 80% sequence identity with any one of SEQ ID NO:19-25. In one embodiment, the hinge sequence comprises an amino acid sequence with at least two conservative amino acid substitutions in any one of SEQ ID NO:19-25. In one embodiment, the linker comprises at least eight amino acid residues. In one embodiment, linker consists of GSA, GGG, or GGSS (SEQ ID NO:11) amino acid sequences. In one embodiment, the linker comprises one or more tandem repeats of GSA, GGG, or GGSS (SEQ ID NO:11) amino acid sequences. In one embodiment, the immunoconjugate lacks a linker sequence. In one embodiment, the immunoconjugate of any one of claims 1-29.

In some embodiments, the disclosure is further drawn to a method for decreasing cancer-related neovascularization in a patient in need thereof, comprising administering to the patient any one of the immunoconjugates or compositions of the disclosure. In one embodiment, a method for slowing the progression of cancer-related neovascularization in a patient in need thereof, comprising administering to the patient a composition of the disclosure.

In some embodiments, administration of the composition is a method for preventing new cancer-related neovascularization in a patient in need thereof, comprising administering to the patient the composition of claim 30. In one embodiment, the administration of the composition is a method for reversing cancer-related neovascularization in a patient in need thereof. In one embodiment, the administration of the composition is a method for treating wet age-related macular degeneration (AMD) in an eye of a patient in need thereof, comprising administering to the patient the composition of claim 30. In some embodiments, treating wet AMD comprises preventing, inhibiting or reversing choroidal neovascularization in the eye of the patient in need of treatment. In one embodiment, a method for preventing, inhibiting, or reversing ocular neovascularization in an eye of a patient in need thereof, comprising administering to the patient the composition. In one embodiment, the method for reversing tumor neovascularization in a patient in need thereof, comprising administering to the patient the composition. In one embodiment, neovascularization is associated with proliferative diabetic retinopathy, wet age-related macular degeneration (AMD), retinopathy of prematurity (ROP), or neovascular glaucoma.

In one embodiment, neovascularization is secondary to proliferative diabetic retinopathy, wet age-related macular degeneration (AMD), retinopathy of prematurity (ROP), or neovascular glaucoma. In one embodiment, neovascularization is choroidal neovascularization. In one embodiment of the methods, the patient has been previously diagnosed with wet age-related macular degeneration (AMD) in the eye. In one embodiment, the choroidal neovascularization is secondary to wet AMD. In one embodiment, the eye of the patient has not been previously treated for choroidal neovascularization or wet AMD. In one embodiment of the methods, the patient has previously been treated for choroidal vascularization with anti-vascular endothelial growth factor (VEGF) therapy, laser therapy or surgery.

In one embodiment, the method comprises intravitreal injection, suprachoroidal injection, or systemic administration. In some embodiments, administration comprises multiple dosing sessions. In one embodiment, the multiple dosing sessions comprise two or more, three or more, four or more, or five or more dosing sessions. In one embodiment, each dosing session is spaced apart by from about 20 days to about 50 days, or from about 20 days to about 40 days, or from about 20 days to about 30 days. In one embodiment, the multiple dosing sessions comprise 12 to 24 dosing sessions. In one embodiment, the administering of the composition comprises intravitreal injection of the composition into the eye of the patient once every 28 days, once every 30 days or once every 35 days. In one embodiment, administering comprises intravenous administration. In one embodiment, administering the composition comprises intratumoral injection.

In some embodiments, administration of the composition results in the patient substantially maintains his or her vision subsequent to the administering step, as measured by losing fewer than 15 letters in a best-corrected visual acuity (BCVA) measurement, compared to the patient's BCVA measurement prior to administering step. In one embodiment, the patient experiences an improvement in vision subsequent to the administering step, as measured by gaining 15 letters in a best-corrected visual acuity (BCVA) measurement, compared to the patient's BCVA prior to the administering step. In one embodiment, the subsequent to the administering step, as measured by fluorescein angiography or optical coherence tomography, the CNV area is reduced in the eye of the patient, as compared to the CNV area prior to the administering step. In one embodiment, the CNV area is reduced by at least about 10%, at least about 20%, at least about 30%, at least about 40%, or at least about 50%. In one embodiment, subsequent to the administering step, the retinal thickness of the eye of the patient is reduced in the eye of the patient, as compared to the retinal thickness of the eye prior to the initiation of treatment.

In one embodiment, administration of the composition results in reductions in retinal thickness by at least about 50 μm, at least about 100 μm, at least about 150 μm, at least about 175 μm, at least about 200 μm, at least about 225 μm or at least about 250 μm. In one embodiment, the retinal thickness is reduced by at least about 10%, at least about 20%, at least about 30%, at least about 40% or at least about 50%. In one embodiment, the decreased retinal thickness is decreased central retinal subfield thickness (CST), decreased center point thickness (CPT), or decreased central foveal thickness (CFT). In one embodiment, the method is further comprising measuring the intraocular pressure (IOP) in the eye of the patient prior to each intravitreal or suprachoroidal injection. In one embodiment, the method is further comprising measuring the IOP in the eye of the patient about 20 minutes, about 30 minutes, about 40 minutes, about 50 minutes or about 1 hour after each intravitreal or suprachoroidal injection. In one embodiment, the method further comprises measuring the IOP in the eye of the patient about 20 minutes, about 30 minutes, about 40 minutes, about 50 minutes or about 1 hour prior to each intravitreal or suprachoroidal injection. In one embodiment, IOP is measured via tonometry.

In one embodiment, the method of administering the immunoconjugate further comprises administering an effective amount of a neovascularization inhibitor to the patient. In one embodiment, the neovascularization inhibitor is present in the same composition as the effective amount of the immunoconjugate. In one embodiment, the neovascularization inhibitor is present in a different composition than the effective amount of the immunoconjugate. In one embodiment, the wherein the neovascularization inhibitor is a vascular endothelial growth factor (VEGF) inhibitor, a VEGF receptor inhibitor, a platelet derived growth factor (PDGF) inhibitor or a PDGF receptor inhibitor. In one embodiment, the neovascularization inhibitor is ranibizumab. In one embodiment, the dosage of ranibizumab is from about 0.2 mg to about 1 mg. In one embodiment, the dosage of ranibizumab is 0.3 mg or 0.5 mg. In one embodiment, ranibizumab is administered to the eye of the patient via an intravitreal injection. In one embodiment, the composition comprising the effective amount of the neovascularization inhibitor is administered to the eye of the patient via an intravitreal injection. In one embodiment, the composition comprising the effective amount of the neovascularization inhibitor is administered at each of the multiple dosing sessions.

In one embodiment, the immunoconjugate composition comprises a mixture of both one-armed and two-armed immunoconjugates, wherein the one-armed and two-armed immunoconjugates are present in a ratio of: 1:1, 1:5, 1:10, 1:25, 1:50, 1:100, 100:1, 50:1, 25:1, 10:1, or 5:1. In one embodiment, the immunoconjugate factor VII protein is a human factor VII protein. In some embodiments, the disclosure is drawn to a formulation comprising an immunoconjugate further comprising a pharmaceutically acceptable excipient. In one embodiment, the formulation further comprises ranibizumab. In one embodiment, the formulation further comprises an arginine solution. In one embodiment, the formulation further comprises one or more of the following: HEPES solution, sodium chloride, calcium chloride, polysorbate-80, and arginine solution.

In some embodiments, cancer-related neovascularization is associated with: melanoma, renal cancer, prostate cancer, breast cancer, ovarian cancer, brain cancer, neuroblastoma, pancreatic cancer, bladder cancer, liver cancer, ocular melanoma, lung cancer, endometrial cancer, stomach cancer, and lymphatic cancer. In one embodiment, the neovascularization inhibitor is administered simultaneously. In one embodiment, the neovascularization inhibitor is administered serially. In one embodiment, the neovascularization inhibitor is aflibercept.

In some embodiments, the compositions of the disclosure result in decreases in pro-inflammatory cytokine signaling. In one embodiment, the composition decreases pro-inflammatory cytokine signaling at least 1.5 fold greater than a two-armed immunoconjugate dimer. In one embodiment, the pro-inflammatory cytokine is IL-8 or GM-CSF.

In some embodiments, the administration of compositions of the disclosure results in decreases in pro-inflammatory cytokine signaling. In some embodiments, at least 1.5 fold greater than a two-armed immunoconjugate dimer. In some embodiments, the pro-inflammatory cytokine is IL-8 or GM-CSF. In one embodiment, the production of one-armed immunoconjugate results in a composition substantially free of the two-armed immunoconjugate. A composition comprising a one-armed immunoconjugate, wherein the composition is substantially free of a two-armed immunoconjugate.

### BRIEF DESCRIPTION OF THE FIGURES

FIG. 1 is a non-limiting graphical representation of one embodiment of a one-armed ICON-1.5 immunoconjugate of the present invention, and one embodiment of a two-armed ICON-1.0 immunoconjugate of the present invention.

FIG. 2A and FIG. 2B are non-limiting graphical representations of multiple embodiments of immunoconjugates of the present disclosure. FIG. 2A presents two-armed FVII-Fc fusion protein variants, and FIG. 2B presents one-armed FVII-Fc fusion protein variants.

FIG. 3A and FIG. 3B are visual representations of data from the characterization of ICON-1.5 one-armed FVH-Fc variant with regard to cell-based binding affinity (FIG. 3A), and antibody-dependent cell-mediated cytotoxicity (ADCC) (FIG. 3B).

FIG. 4 is shows the difference in production yield between ICON-1 and ICON-1.5.

FIG. 5 is a diagram of an exemplary immunoconjugate embodiment of this invention, wherein the Fc dimer comprises the knob-hole mutations.

FIG. 6A and FIG. 6B are visual representations of data from the characterization of CHO-S derived ICON-1 versus CHO-S derived ICON-1.5 with regard to cell-based binding affinity (FIG. 6A), and antibody-dependent cell-mediated cytotoxicity (ADCC) (FIG. 6B).

FIG. 7A and FIG. 7B are visual representations of data from the characterization of HEK293 derived ICON-1.5 missing the GGSS (SEQ ID NO:11) linker vs HEK-293 derived ICON-1.5 with the GGSS (SEQ ID NO:11) linker; with regard to cell-based binding affinity (FIG. 7A), and antibody-dependent cell-mediated cytotoxicity (ADCC) (FIG. 7B).

FIG. 8 is a visual representation of the constructs utilized in producing immunoconjugates of the present invention, which is accompanied by two replicate Western blot transfers of the supernatant collected from the cells comprising said constructs. The blot in the left of the figure utilized Anti-FVII antibody and the blot on the right of the figure utilized Anti-human IgG1 Fc. The legend recites the GGSS (SEQ ID NO:11) linker attached to the DKTHTCPPCP (SEQ ID NO:20) modified Fc hinge.

FIG. 9 is a size exclusion chromatogram of the proteins isolated from the cells expressing the constructs from the aforementioned figure. (see Example 1), specifically demonstrating that the major peak in the sample, labelled as “Monomer” comprises ICON-1.5 immunoconjugates, demonstrating effective isolation and purification of immunoconjugates of the present invention.

FIG. 10 depicts a binding assay and an antibody-dependent cell-mediated cytotoxicity (ADCC) reporter assay for CHO and 293-derived ICON-1.5 immunoconjugate molecules. The left panel is the binding assay, and the right panel is the ADCC reporter assay. The GGSS linker corresponds to SEQ ID NO:11.

FIG. 11 depicts a binding assay and an antibody-dependent cell-mediated cytotoxicity (ADCC) reporter assay for BHK and 293-derived ICON-1.5 immunoconjugate molecules. The left panel is the binding assay, and the right panel is the ADCC reporter assay.

FIG. 12 depicts a cell-based Factor Xa conversion assay utilizing an FXa fluorogenic substrate to determine whether ICON-1 and/or ICON-1.5 interfere with the coagulation response as a result of FVIIa-induced FX activation.

FIG. 13 is a table containing the data from a recombinant TF Factor Xase assay which indicates that ICON-1 and ICON-1.5 share similar inhibitory activities with regard to the ability of recombinant FVIIa to interact with three forms of TF in the in vitro TF FXase assay.

FIG. 14 depicts a secondary antibody-drug conjugate cytotoxicity (ADC) assay with ICON-1 from BHK and ICON-1.5 with GGSS linker (SEQ ID NO:11) from 293 cells. The left panel utilizes an anti-Fc Fab fragment coupled to the anti-tubulin agent MMAF as the secondary antibody. The right panel utilizes an anti-Fc Fab fragment coupled to the DNA intercalator PNU-159268.

FIG. 15 depicts ADC assays with ICON-1.5 from 293 and ICON-1 from BHK. The left panel utilizes the tubulin inhibitor MMAF in the epidermoid carcinoma cell line A431. The right panel utilizes the tubulin inhibitor MMAF in the pancreatic adenocarcinoma cell line BxPC3.

FIG. 16 depicts ADC assays with ICON-1.5 from 293 and ICON-1 from BHK, in which the secondary antibody is conjugated with the tubulin inhibitor MMAF in the triple negative breast carcinoma cell line MDA-MB-231.

FIG. 17 depicts the effect of ICON-1 produced in BHK and ICON-1.5 produced in 293 on FVIIa-induced cell-signaling. The left panel measures IL-8. The right panel measures GM-CSF.

FIG. 18 depicts the data from a xenograft study performed in female athymic nude mice (Crl:NU(NCr)-Foxn1nu, Charles River), in which the potential effect of agents directed against TF on in vivo tumor growth was evaluated.

### DETAILED DESCRIPTION OF THE INVENTION

The term “a” or “an” may refer to one or more of that entity, i.e. can refer to plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

Reference throughout this specification to “one embodiment”, “an embodiment”, “one aspect”, or “an aspect” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

As used herein, in particular embodiments, the terms “about” or “approximately” when preceding a numerical value indicates the value plus or minus a range of 10%.

As used herein, in particular embodiments, the term “substantially free” is to be understand to be 95% free or more. For example, if composition X is substantially free of molecule Y, then composition X is understood to be at least 95% free of molecule Y. Composition X contains less than 5% of molecule Y.

The abnormal growth of existing blood vessels and the creation of new blood vessels (referred to herein collectively as neovascularization), is observed in a variety of diseases, typically triggered by the release of specific growth factors for vascular endothelial cells. Neovascularization can enable solid tumor growth and metastasis, cause visual malfunction in ocular disorders, promote leukocyte extravasation in inflammatory disorders, and/or influence the outcome of cardiovascular diseases such as atherosclerosis.

Provided herein are immunoconjugate molecules comprising a targeting domain (mutated Factor VIIa (FVIIa) protein; referred to herein interchangeably as FVII) and an effector domain (Fc domain), wherein the targeting domain and the effector domains are conjugated. In some embodiments, the immunoconjugate is a one-armed variant (referred to herein interchangeably as ICON-1.5) and comprises a 2 Fc effector moieties of an immunoglobulin (e.g. IgG), wherein one of the Fc moieties is conjugated to a mutated FVIIa, and the two IgG Fc monomers are dimerized.

The immunoconjugate molecules provided herein target and bind to Tissue Factor (TF) in diseased tissue, tumors, and the supporting stroma (e.g. vasculature, infiltrating mononuclear cells) and also bind Fc receptors. In some embodiments, the immunoconjugate molecules comprise a mutated factor VII protein (interchangeably referred to herein as FVII domain FVII moiety) and two immunoglobulin Fc proteins (interchangeably referred to herein as Fc domains Fc moieties, and Fc effector moieties). In other embodiments, the immunoconjugate molecules comprise a dimer of a mutated factor VII protein and an immunoglobulin Fc protein. The VII protein is a targeting domain, and the Fc protein is the effector domain, wherein each VII protein is conjugated to the Fc protein. In the instances where only a single FVII protein is present, the FVII protein is conjugated only to one of the Fc proteins. The targeting domain of the immunoconjugate dimers comprise a mutated FVIIa protein. The effector domain of the immunoconjugate dimers comprise a Fc effector domain of an IgG1 immunoglobulin. Immunoconjugate molecules may comprise a single targeting domain conjugated to a dimerized effector domain, or may comprise two targeting domains conjugated to two effector domains.

As provided herein, the immunoconjugates bind to TF, but do not initiate or exhibit decreased initiation of the clotting cascade. The immunoconjugates comprising the mutated FVIIa protein are designed such that FVIIa's normal role to initiate the clotting cascade does not occur or is reduced.

Provided herein are methods of using the immunoconjugate molecules in treating a patient having a disease associated with pathological neovascularization including, but not limited to atherosclerosis, rheumatoid arthritis, ocular melanoma, BRAF-mutated melanoma, solid tumor, primary or metastatic solid tumors (including but not limited to melanoma, renal, prostate, breast, triple-negative breast, ovarian, brain, neuroblastoma, head and neck, pancreatic, bladder, endometrial and lung cancer), diabetic macular edema (DME), macular edema following retinal vein occlusion (RVO), proliferative diabetic retinopathy, wet age-related macular degeneration (AMD), retinopathy of prematurity (ROP), and neovascular glaucoma.

### Immunoconjugates

Provided herein are immunoconjugates that target and bind to TF in diseased tissue, tumors, and the supporting stroma (e.g. vasculature, infiltrating mononuclear cells), and also bind Fc receptors.

The immunoconjugates described herein comprise a targeting domain and an effector domain wherein the targeting domain and the effector domain are conjugated. In one embodiment, the effector and targeting domain are conjugated together by a hinge domain (interchangeably referred to herein as a hinge region, hinge moiety, or simply hinge). The hinges are provided in greater detail below. As presented herein, in some embodiments, the effector domain is inclusive of a hinge region. In other embodiments, the effector domain does not include a hinge region. In some embodiments, the conjugation further comprises the inclusion of a linker. The linker is provided in greater detail below. The targeting domain of the immunoconjugate comprises a mutated FVIIa protein (tissue factor targeting domain). The effector domain of the immunoconjugate comprises a Fc effector moiety of an IgF1 immunoglobulin. In one embodiment, the targeting domain is a mutated human FVIIa protein and the effector domain is a huma Fc effector moiety of an IgG1 immunoglobulin. In another embodiment the targeting domain is a mutated human FVIIa protein and the effector domain is a non-huma Fc effector moiety of an IgG1 immunoglobulin. In one embodiment the targeting domain is a non-mutated human FVIIa protein and the effector domain is a huma Fc effector moiety of an IgG1 immunoglobulin. In one embodiment the targeting domain is a non-mutated human FVIIa protein and the effector domain is a non-human (from the same species as the targeting domain) Fc effector moiety of an IgG1 immunoglobulin. In one embodiment the targeting domain is a non-mutated human FVIIa protein and the effector domain is a non-human (from a different species as that of the targeting domain) Fc effector moiety of an IgG1 immunoglobulin. In some embodiments, the Fc is from an isotype other than IgG1.

As provided herein, the immunoconjugate binds to TF, but (1) does not initiate, or (2) exhibits deceased initiation of the clotting cascade. The immunoconjugate comprising the mutated FVIIa protein is designed such that FVIIa's normal role to initiate the clotting cascade does not occur or is reduced.

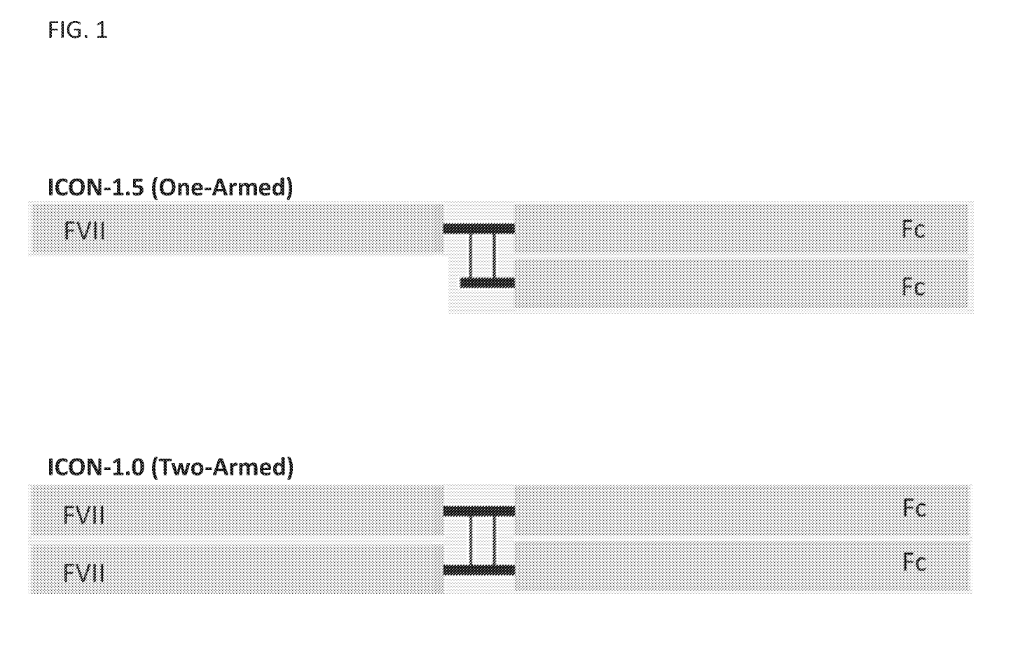

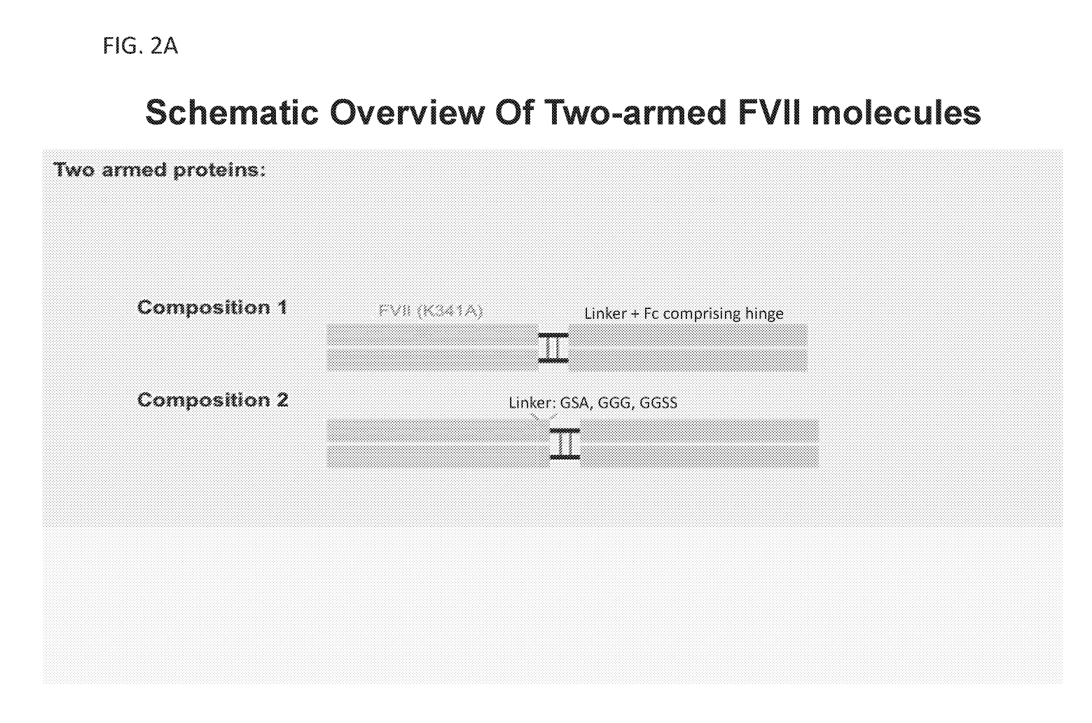

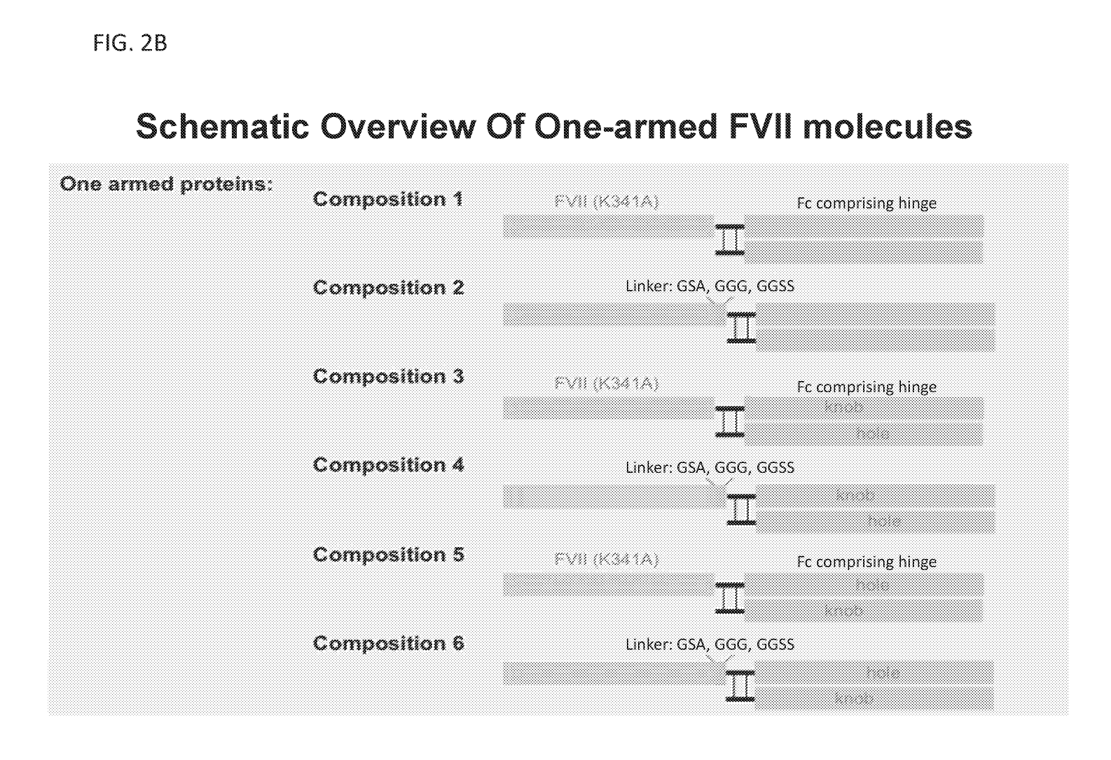

As used herein, “immunoconjugate” or “immunoconjugates” refer to two types of conjugated or fused proteins: (1) ICON-1.5, a one-armed FVII-Fc fusion protein comprising two dimerized immunoglobulin (Ig) Fc monomers, and a mutated FVII protein, wherein the mutated FVII protein is fused to only one of the Fc monomers; and (2) ICON-1, a two-armed FVII-Fc fusion protein comprising two dimerized immunoglobulin (Ig) Fc monomers fused to two mutated FVII proteins (See FIG. 1 and FIG. 2B).

ICON-1.5 is manufactured as a homogenous molecule without impurities such as the presence of un-conjugated FVII or Fc (monomeric or dimeric), or monomeric FVII fused to Fc. In this regard, the production of ICON-1.5 provides significant advantages in the manufacturing environment, reducing the number of products of interest that are not ICON-1.5.

ICON-1.5 is less prone to self-aggregation than ICON-1, resulting in an ease of manufacturability. ICON-1.5 and ICON-1 share similar degrees of (1) binding and ADCC activity, and (2) FXa conversion; as presented in greater detail below.

In some embodiments, the one-armed ICON-1.5 immunoconjugate exhibits a substantially greater inhibitory effect on cytokine signaling than the two-armed ICON-1. In some embodiments, administration of the one-armed immunoconjugate decreases pro-inflammatory cytokine signaling. In some embodiments, the administration of a one-armed immunoconjugate decreases pro-inflammatory cytokine signaling by 1.1, 1.2, 1.3, 1.4, 1.5, 1.6, 1.7, 1.8, 1.9, 2.1, 2.2, 2.3, 2.4, 2.5, 2.6, 2.7, 2.8, 2.9, 3, 3.5, 4, 4.5, 5, 6, 7, 8, 9, 10, 11, 12, 13, 14, 15, 16, 17, 18, 19, 20, 21, 22, 23, 24, 25, 50, or 100-fold greater than a two-armed immunoconjugate decreases pro-inflammatory cytokine signaling. In some embodiments, the administration of a one-armed immunoconjugate decreases pro-inflammatory cytokine signaling by about 1.1, about 1.2, about 1.3, about 1.4, about 1.5, about 1.6, about 1.7, about 1.8, about 1.9, about 2.1, about 2.2, about 2.3, about 2.4, about 2.5, about 2.6, about 2.7, about 2.8, about 2.9, about 3, about 3.5, about 4, about 4.5, about 5, about 6, about 7, about 8, about 9, about 10, about 11, about 12, about 13, about 14, about 15, about 16, about 17, about 18, about 19, about 20, about 21, about 22, about 23, about 24, about 25, about 50, or about 100-fold greater than a two-armed immunoconjugate decreases pro-inflammatory cytokine signaling.

In one exemplary embodiment, the pro-inflammatory cytokine is IL-8. In another exemplary embodiment, the pro-inflammatory cytokine is GM-CSF.

In some embodiments, the Fc region includes the native hinge region, and the immunoconjugate is fused directly between the FVII protein and hinge region of the Fc region. In other embodiments, the FVII-Fc immunoconjugate is separated by a linker region. In some embodiments, the hinge region and/or the linker region is absent from the immunoconjugate.

FIG. 1 and FIG. 2A provide the generalized structure of multiple non-limiting embodiments of a two-armed FVII-Fc protein, while FIG. 2B provide the generalize structure of multiple non-limiting embodiments of a one-armed FVII-Fc protein.

In one embodiment, the immunoconjugate binds to TF expressed on cancer cells. In a further embodiment, the immunoconjugate binds to cancer cells or other cells overexpressing or aberrantly expressing TF.

In some embodiments, the immunoconjugate is post-translationally modified. Post-translational modification includes but is not limited to: myristoylation, glypiation, palmitoylation, prenylation, lipoylation, acylation, alkylation, butrylation, gamma-carboxylation, glycosylation (N-glycosylation, O-glycosylation, fucosylation, and mannosylation), propionylation, succinylation, and sulfation.

In some embodiments, the compositions of the present disclosure, which are utilized in administering to patients, include mixtures comprising both one-armed and two-armed immunoconjugates. In some embodiments the composition comprises one-armed immunoconjugates and two-armed immunoconjugates at a ratio of about 1:1000, 1:900, 1:800, 1:700, 1:600, 1:500, 1:400, 1:300, 1:200, 1:150, 1:100, 1:95, 1:90, 1:85, 1:80, 1:75, 1:70, 1:65, 1:60, 1:55, 1:50, 1:45, 1:40, 1:35, 1:30, 1:25, 1:20, 1:15, 1:14, 1:13, 1:12, 1:11, 1:10, 1:9, 1:8, 1:7, 1:6, 1:5, 1:4, 1:3, 1:2, or 1:1. In some embodiments the composition comprises two-armed immunoconjugates and one-armed immunoconjugates at a ratio of about 1:1000, 1:900, 1:800, 1:700, 1:600, 1:500, 1:400, 1:300, 1:200, 1:150, 1:100, 1:95, 1:90, 1:85, 1:80, 1:75, 1:70, 1:65, 1:60, 1:55, 1:50, 1:45, 1:40, 1:35, 1:30, 1:25, 1:20, 1:15, 1:14, 1:13, 1:12, 1:11, 1:10, 1:9, 1:8, 1:7, 1:6, 1:5, 1:4, 1:3, 1:2, or 1:1.

In some embodiments, the composition comprises one-armed immunoconjugates at a relative abundance of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%/a, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 700%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% relative to the abundance of two-armed immunoconjugates.

As provided throughout, in embodiments described herein, an immunoconjugate comprising a tissue factor targeting domain comprising a mutated Factor VIIa domain is provided. The targeting domain comprises a mutated Factor VIIa that has been mutated to inhibit (or reduce) initiation of the coagulation pathway without reducing binding affinity to tissue factor. In one embodiment, the mutation in human Factor VIIa is a single point mutation at residue 341. In a further embodiment, the mutation in human Factor VIIa is from Lys341 to Ala341. In other embodiments, where the mutant Factor VIIa is from a non-human species, it can comprise a mutation that corresponds to a mutation at residue 341 of the human Factor VIIa. Other mutations that inhibit the coagulation pathway are encompassed by the immunoconjugates provided herein. The mutated Factor VIIa domain (also referred to as the TF targeting domain), in the aspects provided herein, binds tissue factor with high affinity and specificity, but does not initiate coagulation, or minimizes coagulation normally associated with tissue factor binding.

The effector domain of the immunoconjugates provided herein comprise an Fc effector moiety of an IgG1 immunoglobulin. In one embodiment, the effector domain mediates both complement and natural killer (NK) cell cytotoxicity pathways. In one embodiment, cytotoxicity of immunologic cells such as NK cells and macrophages are activated by activating the Fc effector moiety when bound to Fc receptors present on cells of the immune system. The IgG1 Fc effector domain can trigger a cytolytic response against cells which bind the immunoconjugate, by the natural killer (NK) cell and complement pathways. In one embodiment, the IgG1 Fc effector domain comprises both the CH2 and CH3 regions of the IgG1 Fc region.

The reaction between FVIIa and TF is species-specific (Janson et al., 1984; Schreiber et al., 2005; Peterson et al., 2005): murine FVII appears to be active in many heterologous species including rabbits, pigs and humans, whereas human FVIIa is appreciably active in humans, non-human primates, dogs, rabbits, and pigs. Conversely, the human IgG Fc domain is active in both humans and mice. Accordingly, depending on the patient, the immunoconjugate is constructed using targeting and effector domains derived from the corresponding species, or from a species that is known to be active in the patient. For example, in the human treatment methods provided herein, the mutated tissue factor targeting domain can be derived from human Factor VIIa conjugated to an effector domain comprising the Fc region of a human IgG1 immunoglobulin.

In one embodiment, the immunoconjugate is present in a composition comprising about 0.01, about 0.05, about 0.1, about 0.15, about 0.2, about 0.25, about 0.3, about 0.35, about 0.4, about 0.45, about 0.5, about 0.55, about 0.6, about 0.7, about 0.8, about 0.9, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, or about 25 mM HEPES or other pharmaceutically acceptable buffer.

In one embodiment, the immunoconjugate is present in composition comprising 0.01, 0.05, 0.1, 0.15, 0.2, 0.25, 0.3, 0.35, 0.4, 0.45, 0.5, 0.55, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, or 25 mM HEPES or other pharmaceutically acceptable buffer.

In one embodiment, the immunoconjugate is present in a composition comprising about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 175, about 180, about 185, about 190, about 195, about 200, about 205, about 210, about 215, or about 220 mM NaCl.

In one embodiment, the immunoconjugate is present in a composition comprising 40, 45, 50, 55, 60, 65, 70, 75, 80, 85, 90, 95, 100, 105, 110, 115, 120, 125, 130, 135, 140, 145, 150, 155, 160, 175, 180, 185, 190, 195, 200, 205, 210, 215, or 220 mM NaCl.

In one embodiment, the immunoconjugate is present in a composition comprising about 5, about 10, about 15, about 20, about 25, about 30, about 35, about 40, about 45, about 50, about 55, about 60, about 65, about 70, or about 75 mM Arginine, Glycine, Histidine, or any other naturally occurring amino acid. In some embodiments, the composition comprises combinations of amino acids, e.g. arginine and histidine.

In one embodiment, the immunoconjugate is present in a composition comprising 5, 10, 15, 20, 25, 30, 35, 40, 45, 50, 55, 60, 65, 70, or 75 mM Arginine, Glycine, Histidine, and/or any other naturally occurring amino acid.

In one embodiment, the immunoconjugate is present in a composition with a pH of about 7.0, about 7.05, about 7.10, about 7.15, about 7.2, about 7.25, about 7.3, about 7.35, about 7.4, about 7.45, about 7.5, about 7.55, about 7.6, about 7.65, about 7.7, about 7.75, or about 7.75.

In one embodiment, the immunoconjugate is present in a composition with a pH of 7.0, 7.05, 7.10, 7.15, 7.2, 7.25, 7.3, 7.35, 7.4, 7.45, 7.5, 7.55, 7.6, 7.65, 7.7, 7.75, or 7.75.

In one embodiment, the immunoconjugate is present in a composition comprising about 0.001%, about 0.0015%, about 0.002%, about 0.0025%, about 0.003%, about 0.0035%, about 0.004%, about 0.0045%, about 0.005%, about 0.0055%, about 0.006%, about 0.0065%, about 0.007%, about 0.0075%, about 0.0085%, about 0.009%, about 0.0095%, about 0.01%, about 0.015%, about 0.02%, about 0.025%, about 0.03%, about 0.035%, about 0.04%, about 0.045%, or about 0.05% polysorbate-80.

In one embodiment, the immunoconjugate is present in a composition comprising 0.001%, 0.0015%, 0.002%, 0.0025%, 0.003%, 0.0035%, 0.004%, 0.0045%, 0.005%, 0.0055%, 0.006%, 0.0065%, 0.007%, 0.0075%, 0.0085%, 0.009%, 0.0095%, 0.01%, 0.015%, 0.02%, 0.025%, 0.03%, 0.035%, 0.04%, 0.045%, or 0.05% polysorbate-80.

In one embodiment, the immunoconjugate is present in a composition comprising about 0.05, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, about 10, about 11, about 12, about 13, about 14, about 15, about 16, about 17, about 18, about 19, or about 20 CaCl₂.

In one embodiment, the immunoconjugate is present in a composition comprising 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, or 20 CaCl₂.

### Tissue Factor (TF)

TF is cell surface receptor for the serine protease factor VIIa. While TF is involved with a variety of functions at the cellular and organismal level, it is most widely known for its role in blood coagulation. TF complexed with factor VIIa activates factor IX and catalyzes the conversion of factor X to active factor Xa, a protease necessary for the common pathway of coagulation. TF is expressed by many types of cancers. TF-dependent activation has been implicated in cancer-associated thrombosis and metastasis. In addition to possessing procoagulant activity, TF has cell signaling properties. The formation of the TF activated-FVII complex on the surface of tumor cells leads to cleavage and activation of the G-protein-coupled receptor PAR2. The TF-FVIIa-PAR2 signaling pathway promotes tumor growth. See van den Berg. 2012. Blood. 119(4):924-932; and Kasthuri. 2009. J. Clin. Oncol. 27(29):4834-4838).

In one aspect of the present invention, methods for treating a patient having a disease or disorder associated with aberrant expression of TF, such as cancer, hematogenous metastasis, cancer-associated thromboembolism, etc., are provided. As described herein, administration may be local or systemic, depending upon the type of disease or disorder involved in the therapy.

### Factor VII (FVII)

Factor VII (FVII), sometimes referred to as the TF targeting domain, binds TF with high affinity and specificity. FVII is one of the proteins that causes blood to clot in the coagulation cascade. FVII is involved in the initiation of the coagulation cascade by binding to TF and activating, which in turn initiates the coagulation cascade. Certain mutations to FVII, can result in a FVII that retains its ability to bind TF, but does not initiate coagulation or exhibit coagulation normally associated with the binding to TF as compared to the non-mutated form.

The reaction between FVII and TF has been reported to be species-specific (Janson et al., 1984; Schreiber et al., 2005; Peterson et al., 2005). Murine FVII appears to be active in many heterologous species including rabbit, pig and human, whereas human FVII has been reported appreciably active in human, dog, rabbit and pig. Accordingly, depending on the patient, the immunoconjugate is constructed using targeting and effector domains derived from the corresponding species, or from a species that is known to be active in the patient. For example, in the human treatment methods provided herein, the mutated tissue factor targeting domain may be derived from human Factor VII conjugated to an effector domain comprising the Fc region of a human IgG1 immunoglobulin. For example, in one embodiment, the mutated FVII moiety of the immunoconjugate comprises the amino acid sequence of SEQ ID NO:34. In one embodiment, the immunoconjugate comprises a targeting domain (mutated FVII domain) joined to an effector domain (human IgG1 Fc) via a linker and/or hinge region.

In some embodiments, the targeting domain comprises a mutated Factor VII that has been mutated to inhibit initiation of the coagulation pathway without reducing binding affinity to tissue factor. In one embodiment, the mutation in Factor VII is a single point mutation at residue 341. In a further embodiment, the mutation is from Lys341 to Ala341. In a further embodiment, the mutation is from Ser344 to Ala344. However, other mutations that inhibit the coagulation pathway are encompassed by the immunoconjugates provided herein.

In some embodiments, the immunoconjugates comprise FVII, wherein the FVII comprises the heavy chain and the light chain. In one embodiment, the FVII comprises only the heavy chain. In a further embodiment, the FVII consists of a fragment of the heavy chain. In one embodiment, the FVII comprises only the light chain. In a further embodiment, the FVII consists of a fragment of the light chain. In some embodiments, the light chain fragment consists of amino acid residues 1-120, 1-125, 1-130, 1-135, 1-140, 1-150, 1-155, 1-160, 1-165, 1-170 of the FVII light chain. In some embodiments, the light chain fragment consists of amino acid residues 1-145, 1-146, 1-147, 1-148, 1-149, 1-150, 1-151, 1-152, 1-153, 1-154, 1-155, 1-156, 1-157, 1-158, 1-159, or 1-160.

In some embodiments, one-armed and two-armed FVII-Fc immunoconjugates of the present disclosure exhibit a reduced coagulation response, in vitro or in vivo, as compared to immunoconjugates or compositions thereof comprising a wild-type Factor VII domain or the Factor VII domain encoded by NCBI Accession AF272774. In some embodiments, one-armed and two-armed FVII-Fc immunoconjugates of the present disclosure do not exhibit a coagulation response in vitro or in vivo.

In some embodiments, the FVII is post-translationally modified. Post-translational modification includes: myristoylation, glypiation, palmitoylation, prenylation, lipoylation, acylation, alkylation, butrylation, gamma-carboxylation, glycosylation (N-glycosylation, O-glycosylation, fucosylation, and mannosylation), propionylation, succinylation, and sulfation.

In some embodiments, the immunoconjugates may comprise an FVII targeting domain selected from: SEQ ID NO:12. SEQ ID NO:33, or SEQ ID NO:34.

### Fc Proteins

Fragment crystallizable (Fc) proteins are antibody fragments, generally comprising the antibody tail region. Fc proteins naturally interact with cell surface receptors (Fc receptors) and proteins of the complement system, and this property allows the Fc regions to activate the host immune system. The immunoglobulin Fc effector domain of the immunoconjugate can trigger a cytolytic response against cells which bind the immunoconjugate. The cytolytic response can be triggered, for example, by the natural killer (NK) cell and complement pathways. In some embodiments, the Fc effector domain comprises both the CH2 and CH3 regions. In some embodiments, the Fc effector domain comprises only the CH2 region. In some embodiments, the Fc effector domain comprises only the CH3 region. In some embodiments, the Fc is from another immunoglobulin, i.e., IgM, IgE, IgA, etc. In some embodiments, the IgG Fc region is modified in an immunoconjugate described herein. In one embodiment, the IgG Fc region comprises SEQ ID NO:13. In one embodiment, the IgG Fc comprises SEQ ID NO:14, which comprises a protA mutation, relative to SEQ ID NO:13. In one embodiment, the IgG Fc region comprises SEQ ID NO:15, which comprises a T366Y mutation, relative to SEQ ID NO:13. The T366Y mutation of Fc is referred to as a knob mutation. In one embodiment, the IgG Fc region comprises SEQ ID NO:17, which comprises a Y407T mutation, relative to SEQ ID NO:13. The Y407T mutation of Fc is referred to as a hole mutation. In one embodiment, the IgG Fc region comprises SEQ ID NO:26, SEQ ID NO:27, SEQ ID NO:28, SEQ ID NO:29, SEQ ID NO:30, SEQ ID NO:31, or SEQ ID NO:32. The knob and hole mutations, when expressed together creating the immunoconjugate comprising the knob/hole Fc heterodimer, result in an increased yield of wholly formed Fc-containing immunoconjugate molecules. See Ridgway et al. (1996. Protein Engineering. 9(7):617-621). In some embodiments, S354C or T366W are viable knob mutations, and Y349C, T366S, L368A, and Y407V are viable hole mutations. While the aforementioned knob and hole mutations contribute to the yield, by means of an increased stability, Klein et al. (2012. MAbs. 4(6):653-663) sets forth additional modifications for stabilizing the Fc. In the absence of a specific reference residue, reference to a hole mutation is to be interpreted as Y407T, and reference to a knob mutation is to be interpreted as T366Y. It is also understood that the residue number in other Fc variants may be different, in which case references is made to the residue corresponding to the Y407 (in the case of the hole mutation) and T366 (in the care of the knob mutation).

In one embodiment, the IgG Fc region comprises SEQ ID NO:14 or SEQ ID NO:28, which comprises the protA mutation. In one embodiment, the IgG Fc region comprises SEQ ID NO:16 or SEQ ID NO:30, which comprise the T366Y knob mutation and the protA mutation. In one embodiment, the IgG Fc region comprises SEQ ID NO:18 or SEQ ID NO:32, which comprises the Y407T hole mutation and the protA mutation. Other mutations, including knob and hole as well as mutations that change or improve effector function may be used in the immunoconjugate. In some embodiments, the effector domain comprising the Ig Fc region of the immunoconjugates provided herein mediates both complement and natural killer (NK) cell cytotoxicity pathways.

In some embodiments, the IgG1 Fc region is substituted for an IgG2, IgG3, or IgG4 region. In some embodiments, the substitution of the IgG1 Fc region with that of another IgG Fc region modulates the effector function. In some embodiments, the substitution of the IgG1 Fc region with that of another IgG Fc region causes an increase in effector function. In some embodiments, the substitution of the IgG1 Fc region with that of another IgG Fc region causes a decrease in effector function. In some embodiments, the substitution of the IgG1 Fc region with that of another IgG Fc results in the modulation of antibody-dependent cell-mediated cytotoxicity (ADCC). In some embodiments, the Fc dimers are homodimers and in other embodiments the Fc dimers are heterodimers.

In some embodiments, the IgG Fc is post-translationally modified. Post-translational modification includes: myristoylation, glypiation, palmitoylation, prenylation, lipoylation, acylation, alkylation, butrylation, gamma-carboxylation, glycosylation (N-glycosylation, O-glycosylation, fucosylation, and mannosylation), propionylation, succinylation, and sulfation. In other embodiments, the IgG Fc modified such that fucosylation is removed. In further embodiments, the removal of fucosylation of Fc increases effector function.

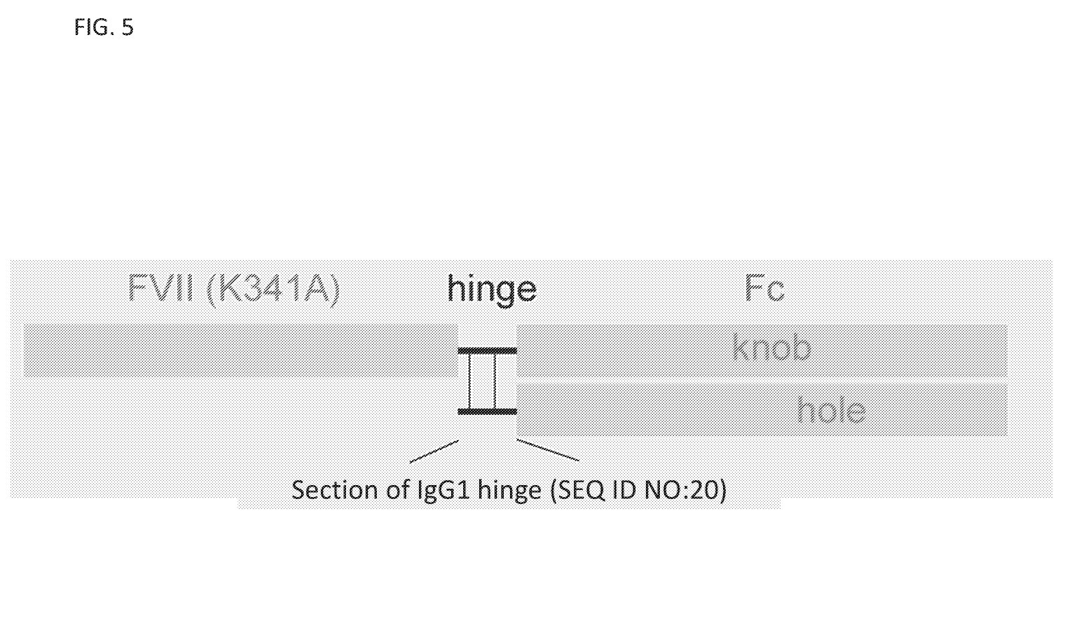

In one embodiment, the IgG Fc regions of the dimer were engineered to be complementary knob-hole mutants, with a knob mutation (SEQ ID NO:15 or 29) occurring in one of the Fc monomers of the dimer and a hole mutation (SEQ ID NO:17 or 31) occurring in the other Fc monomer (See FIG. 5 for one embodiment). In one embodiment, the use of the joint knob-hole Fc mutants results in the preferential production of the one-armed immunoconjugate over the two-armed conjugate. See U.S. Pat. No. 5,731,168. In some embodiments, the protein A mutations are engineered into Fc, Fc knob mutants, Fc hole mutants to facilitate the removal of Fc-only (no FVII fusion) dimer. See U.S. Pat. No. 8,586,713.

In some embodiments, a two-step purification strategy is utilized: (1) a protein A capture step with commonly used MabSelect SuRe resin, followed by (2) size exclusion chromatography (SEC) or anion exchange chromatography to remove two-armed homodimers and Fc-only homodimers.

In some embodiments, the immunoconjugates may comprise an IgG Fc effector domain selected from: SEQ ID NO:13, SEQ ID NO:14, SEQ ID NO:15, SEQ ID NO:16, SEQ ID NO:17, SEQ ID NO:18, SEQ ID NO:26, SEQ ID NO:27, SEQ ID NO:28, SEQ ID NO:29, SEQ ID NO:30, SEQ ID NO:31, and SEQ ID NO:32.

### Hinge Region

The hinge region of immunoglobulins is a flexible amino acid stretch in the heavy chains of immunoglobulins which are a point of origin for linking the heavy chains together by disulfide bonds. The hinge region is a structure that confers both stability and flexibility, which are properties that can be modulated to increase or decrease stability and/and or flexibility. In order to improve the ease of manufacturability of the immunoconjugates, without affecting their binding properties, the hinge regions may be modified. The hinge region of IgG1 comprises cysteine amino acids which form one or more disulfide bonds that result in the dimerization of the IgG1 Fc region. In some embodiments, the hinge region comprises any one of SEQ ID NOs:19-25. See WO2012123586A1 for exemplary Ig hinge regions.

In one embodiment, the hinge region is naturally occurring. In another embodiment the hinge region is not naturally occurring. In one embodiment is of human origin. The hinge region of an IgG1 immunoglobulin, for example the hinge region of the human IgG1 immunoglobulin, in one embodiment, is utilized to link the FVII region to the Fc region in the immunoconjugate described herein.

In one embodiment, the hinge region of the immunoconjugate is the IgG1 hinge region, EPKSCDKTHTCPPCPAPELLGGP (SEQ ID NO:21). In some embodiments, the hinge is selected from any one of SEQ ID NO:19 to 25. In one embodiment, the native IgG1 Fc hinge region is included as the N-terminal portion of the IgG1 Fc of the present disclosure.

In some embodiments, the hinge region includes one or more cysteine amino acids which form one or more disulfide bonds between to monomer chains (e.g., as depicted in FIGS. 1 and 2). In some embodiments, the hinge region is modified. Without wishing to be bound by theory, it is thought that such a modification can aid in the yield of the immunoconjugate. In some embodiments, the hinge region of the immunoconjugate is altered to improve manufacturability of the immunoconjugate without affecting the binding properties to TF or Fc-receptors, all the while maintaining the flexibility of the region.

In one embodiment, the immunoconjugate lacks the hinge region. In some embodiments, the hinge of the immunoconjugates of the present disclosure share at least about 25%, at least about 50%, at least about 60%, at least about 70%, at least about 75%, at least about 80%, at least about 85%, at least about 90%, or at least about 95% sequence identity with the amino acid sequences of any one of SEQ ID NOs:8-11 and 19-25.

In some embodiments, the hinge of the immunoconjugates of the present disclosure comprise conserved amino acid substitutions wherein at least one amino acid residue is substituted for another in the same class, wherein the amino acids are divided into non-polar, acidic, basic, and neutral classes, as follows: non-polar: Ala, Val, Leu, Ile, Phe, Trp, Pro, Met; acidic: Asp, Glu; basic: Lys, Arg, His; neutral: Gly, Ser, Thr, Cys, Asn, Gln, Tyr. In further embodiments, at least two, at least three, at least four, at least five, at least six, at least seven, at least eight, at least 9, or at least 10 conserved amino acid residues are substituted for another in the same class. In some embodiments, the hinge of the immunoconjugate of the present disclosure comprises non-conserved amino acid substitutions are made, wherein the residues do not fall into the same class, for example, a substitution of a basic amino acid for a neutral or non-polar amino acid. In further embodiments, at least two, at least three, at least four, at least five, at least six, at least seven, at least eight, at least nine, or at least ten non-conserved amino acid residues are substituted, wherein the residues do not fall into the same class. In further embodiments, the hinge of the immunoconjugates of the present disclosure are substituted with both conserved and non-conserved amino acid substitutions.

In some embodiments, the hinge of the immunoconjugates of the present disclosure is at least one, at least two, at least three, at least four, at least five, at least six, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acid residues in length.

In some embodiments, the hinge of the immunoconjugates of the present disclosure have deletions of at least one, at least two, at least three, at least four, at least five, or at least six residues from a N- and/or C-terminus naturally occurring hinge sequence. In some embodiments, the hinge of the immunoconjugates of the present disclosure have additions of at least one, at least two, at least three, at least four, at least five, or at least six residues from the N- and/or C-terminus of a naturally occurring hinge. In some embodiments, the immunoconjugate lacks a hinge region, and comprises the hingeless portion of Fc and the FVII. In some embodiments, the immunoconjugate lacks a hinge region and comprises the hingeless portion of the Fc, the FVII, and a linker between the two. In some embodiment, the disulfide bonding to maintain the dimerization is achieved by other means.

In some embodiments, the immunoconjugate may comprise a hinge region selected from: SEQ ID NO:8, SEQ ID NO:9, SEQ ID NO:10, SEQ ID NO:11, or any one of SEQ ID NO:19-25. In some embodiments, the immunoconjugate lacks a hinge region.

### Linker Region

The linker region of the immunoconjugates acts as the bridge or conjugation point between the FVII and the Fc. The composition of the linker region plays a role in manufacturing ease as well as the stability and flexibility of the immunoconjugate. Modulating the linker composition can be done without affecting the binding properties of the immunoconjugate.

In some embodiments, a linker region occurs in the immunoconjugate between the FVII protein and the Fc region. In some embodiments, the linker region comprises one or more of GSA, GGG, or GGSS (SEQ ID NO:11). In one embodiment, the immunoconjugate lacks a linker region.

In some embodiments, the linker regions of GSA, GGG, or GGSS (SEQ ID NO:11) are modified. In some embodiments, the linker of the immunoconjugates of the present disclosure share at least about 25%, at least about 50%, at least about 75% with GSA, GGG, or GGSS (SEQ ID NO:11).

In some embodiments, the linker of the immunoconjugates of the present disclosure comprise conserved amino acid substitutions wherein at least one amino acid residue is substituted for another in the same class, wherein the amino acids are divided into non-polar, acidic, basic, and neutral classes, as follows: non-polar: Ala, Val, Leu, Ile, Phe, Trp, Pro, Met; acidic: Asp, Glu; basic: Lys, Arg, His; neutral: Gly, Ser, Thr, Cys, Asn, Gin, Tyr. In further embodiments, at least two, at least three, at least four, at least five, at least six, at least seven, at least eight, at least 9, or at least 10 conserved amino acid residues are substituted for another in the same class. In some embodiments, the linker of the immunoconjugate of the present disclosure comprises non-conserved amino acid substitutions are made, wherein the residues do not fall into the same class, for example, a substitution of a basic amino acid for a neutral or non-polar amino acid. In further embodiments, at least two, at least three, at least four, at least five, at least six, at least seven, at least eight, at least nine, or at least ten non-conserved amino acid residues are substituted, wherein the residues do not fall into the same class. In further embodiments, the linker of the immunoconjugates of the present disclosure are substituted with both conserved and non-conserved amino acid substitutions.

In some embodiments, the linker of the immunoconjugates of the present disclosure is at least one, at least two, at least three, at least four, at least five, at least six, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acid residues.

In some embodiments, the linker of the immunoconjugates of the present disclosure have deletions of at least one, at least two, at least three, at least four, at least five, or at least six residues from the N- and/or C-terminus. In some embodiments, the linker of the immunoconjugates of the present disclosure have additions of at least one, at least two, at least three, at least four, at least five, or at least six residues from the N- and/or C-terminus.

In some embodiments, the linker may comprise at least 1, 2, 3, 4, 5, 6, 7, 8, 9, or 10 repeats of GSA, GGG, and/or GGSS (SEQ ID NO:11)

In some embodiments, the immunoconjugate comprises a linker region comprising GSA, GGG, or GGSS (SEQ ID NO:11). In one embodiment, the immunoconjugate lacks a linker region. In some embodiments, the immunoconjugate comprises a linker region comprising one or more repeats of GSA, GGG, or GGSS (SEQ ID NO:11).

### Exemplary Immunoconjugates

Exemplary sequences for components of the immunoconjugate dimers described herein are provided in Table 1. Tables 2A and 2B provide exemplary immunoconjugates; these exemplary immunoconjugates by no way limit the genus of immunoconjugates presented within the scope of the disclosure.

In one embodiment described herein, the immunoconjugate is or comprises a protein of SEQ ID NO: 4. In a further embodiment, a monomer of the immunoconjugate is or comprises a protein of SEQ ID NO: 4. In one embodiment, a monomer of the immunoconjugate is encoded by the sequence of SEQ ID NO: 6 or 7.

In one embodiment, the immunoconjugate is or comprises a protein of SEQ ID NO:4. In one embodiment, the immunoconjugate described herein consists of the sequence of SEQ ID NO:4.

In one embodiment, the immunoconjugate is or comprises a protein of SEQ ID NO:5. In one embodiment, the immunoconjugate described herein consists of the sequence of SEQ ID NO:5.

In one embodiment, a monomer of the immunoconjugate is or comprises an FVII selected from SEQ ID NO:1, 2, 12, 33, or 34; and a Fc selected from SEQ ID NO:26-32, and wherein a hinge separates the Fc from the FV, and wherein the hinge is selected from one of SEQ ID NO:19-25 In one embodiment, a linker separates the FVII from the hinge. In one embodiment, the linker separates the FVII from the Fc.

In one embodiment, the targeting domain of the immunoconjugate is or comprises a sequence of SEQ ID NO:1. In one embodiment, the targeting domain of the immunoconjugate consists of a sequence of SEQ ID NO:1.

In one embodiment, the targeting domain of the immunoconjugate is or comprises a sequence of SEQ ID NO:2. In one embodiment, the targeting domain of the immunoconjugate consists of a sequence of SEQ ID NO:2.

In one embodiment, the targeting domain of the immunoconjugate is or comprises a sequence of SEQ ID NO:12. In one embodiment, the targeting domain of the immunoconjugate consists of a sequence of SEQ ID NO:12.

In one embodiment, the targeting domain of the immunoconjugate is or comprises a sequence of SEQ ID NO:33. In one embodiment, the targeting domain of the immunoconjugate consists of a sequence of SEQ ID NO:33.

In one embodiment, the targeting domain of the immunoconjugate is or comprises a sequence of SEQ ID NO:34. In one embodiment, the targeting domain of the immunoconjugate consists of a sequence of SEQ ID NO:34.

In one embodiment, the effector domain of the immunoconjugate comprises the sequence of SEQ ID NO:13. In one embodiment, the effector of the immunoconjugate consists of the sequence of SEQ ID NO:13.

In one embodiment, the effector domain of the immunoconjugate comprises the sequence of SEQ ID NO:14. In one embodiment, the effector domain of the immunoconjugate consists of the sequence of SEQ ID NO:14.

In one embodiment, the effector domain of the immunoconjugate comprises the sequence of SEQ ID NO:15. In one embodiment, the effector domain of the immunoconjugate consists of the sequence of SEQ ID NO:15.

In one embodiment, the effector domain of the immunoconjugate comprises the sequence of SEQ ID NO:16. In one embodiment, the effector domain of the immunoconjugate consists of the sequence of SEQ ID NO:16.

In one embodiment, the effector domain of the immunoconjugate comprises the sequence of SEQ ID NO:17. In one embodiment, the effector domain of the immunoconjugate consists of the sequence of SEQ ID NO:17.

In one embodiment, the effector domain of the immunoconjugate comprises the sequence of SEQ ID NO:18. In one embodiment, the effector domain of the immunoconjugate consists of the sequence of SEQ ID NO:18.

In one embodiment, the effector domain of the immunoconjugate comprises the sequence of SEQ ID NO:26. In one embodiment, the effector domain of the immunoconjugate consists of the sequence of SEQ ID NO:26.

In one embodiment, the effector domain of the immunoconjugate comprises the sequence of SEQ ID NO:27. In one embodiment, the effector domain of the immunoconjugate consists of the sequence of SEQ ID NO:27.

In one embodiment, the effector domain of the immunoconjugate comprises the sequence of SEQ ID NO:28. In one embodiment, the effector domain of the immunoconjugate consists of the sequence of SEQ ID NO:28.

In one embodiment, the effector domain of the immunoconjugate comprises the sequence of SEQ ID NO:29. In one embodiment, the effector domain of the immunoconjugate consists of the sequence of SEQ ID NO:29.

In one embodiment, the effector domain of the immunoconjugate comprises the sequence of SEQ ID NO:30. In one embodiment, the effector domain of the immunoconjugate consists of the sequence of SEQ ID NO:30.

In one embodiment, the effector domain of the immunoconjugate comprises the sequence of SEQ ID NO:31. In one embodiment, the effector domain of the immunoconjugate consists of the sequence of SEQ ID NO:31.

In one embodiment, the effector domain of the immunoconjugate comprises the sequence of SEQ ID NO:32. In one embodiment, the effector domain of the immunoconjugate consists of the sequence of SEQ ID NO:32.

In one embodiment a targeting domain comprising the sequence of SEQ ID NO:12 and is conjugated to an effector domain (inclusive of a hinge region), wherein the effector domain (inclusive of a hinge region) comprises the sequence of SEQ ID NO:13, SEQ ID NO:14, SEQ ID NO:15, SEQ ID NO:16, SEQ ID NO:17, or SEQ ID NO:18.

In one embodiment a targeting domain comprising the sequence of SEQ ID NO:33 and is conjugated to an effector domain (inclusive of a hinge region), wherein the effector domain (inclusive of a hinge region) comprises the sequence of SEQ ID NO:13, SEQ ID NO:14, SEQ ID NO:15, SEQ ID NO:16, SEQ ID NO:17, or SEQ ID NO:18.

In one embodiment a targeting domain monomer comprising the sequence of SEQ ID NO:34 is conjugate to an effector domain dimer, wherein the effector domain comprises the sequence of SEQ ID NO:13, SEQ ID NO:14, SEQ ID NO:15, SEQ ID NO:16, SEQ ID NO:17, or SEQ ID NO:18.

In one embodiment, the elements of the immunoconjugates are modular such that it is contemplated that any one of the effector domains may be conjugated to any one of the targeting domains, and wherein any of the linker sequences may separate the effector domain from the targeting domain. In some embodiments, the hinge sequence may separate the Fc from the FVII or may separate the linker from the Fc.

TABLE 1
Exemplary Sequences
Table 1 - Exemplary Sequences

| Sequence Identifier | Description |
|---|---|
| SEQ ID NO: 1 | Wild-type human FVII amino acid sequence |
| | with signal sequence and propeptide sequence |
| SEQ ID NO: 2 | Wild-type human FVII amino acid sequence |
| | without signal sequence and propeptide |
| | sequence |
| SEQ ID NO: 3 | Homo sapiens FVII active site mutant |
| | immunoconjugate mRNA, complete CDS; |
| | NCBI Accession AF272774 |
| SEQ ID NO: 4 | Homo sapiens FVII active site mutant |
| | immunoconjugate amino acid sequence |
| SEQ ID NO: 5 | Homo sapiens FVII active site mutant |
| | immunoconjugate amino acid sequence, |
| | S344A and A341K (relative to SEQ ID NO: 4) |
| SEQ ID NO: 6 | Homo sapiens FVII active site mutant |
| | immunoconjugate coding sequence |
| SEQ ID NO: 7 | Homo sapiens FVII active site mutant |
| | immunoconjugate coding sequence |
| SEQ ID NO: 8 | Immunoconjugate linker - upper hinge region |
| | amino acid sequence |
| SEQ ID NO: 9 | Mutant immunoconjugate linker - upper |
| | hinge region amino acid sequence; C-terminal |
| | cysteine mutated to serine (relative to SEQ ID |
| | NO: 8) |
| SEQ ID NO: 10 | Mutant immunoconjugate linker - upper |
| | hinge region amino acid sequence; lysine at |
| | position 6 mutated to alanine (relative to SEQ |
| | ID NO: 9) |
| SEQ ID NO: 11 | Mutant immunoconjugate linker - upper |
| | hinge region amino acid sequence; serine at |
| | position 2 mutated to glycine and residues 3-6 |
| | deleted (relative to SEQ ID NO: 9) |
| SEQ ID NO: 12 | Human FVII active site mutant amino acid |
| | sequence |
| SEQ ID NO: 13 | IgG1 Fc amino acid sequence (with lower |
| | hinge) |
| SEQ ID NO: 14 | IgG1 Fc amino acid sequence (with lower |
| | hinge) with protA mutation |
| SEQ ID NO: 15 | IgG1 Fc amino acid sequence with T366Y |
| | knob mutation (relative to SEQ ID NO: 13) |
| SEQ ID NO: 16 | IgG1 Fc amino acid sequence with T366Y |
| | knob mutation (relative to SEQ ID NO: 13) |
| | and protA mutation |
| SEQ ID NO: 17 | IgG1 Fc amino acid sequence with Y407T |
| | hole mutation (relative to SEQ ID NO: 13) |
| SEQ ID NO: 18 | IgG1 Fc region with Y407T hole mutation |
| | (relative to SEQ ID NO: 13) and protA |
| | mutation |
| SEQ ID NO: 19 | Upper hinge region amino acid sequence |
| SEQ ID NO: 20 | Lower hinge region amino acid sequence |
| SEQ ID NO: 21 | IgG1 hinge amino acid sequence |
| SEQ ID NO: 22 | Truncated IgG1 hinge amino acid sequence |
| SEQ ID NO: 23 | Mutated IgG1 hinge amino acid sequence - |
| | N-terminal cysteine mutated to serine (relative |
| | to SEQ ID NO: 21) |
| SEQ ID NO: 24 | Mutated IgG1 hinge amino acid sequence - |
| | N-terminal lysine mutated to alanine (relative |
| | to SEQ ID NO: 23) |
| SEQ ID NO: 25 | IgG4 hinge amino acid sequence |
| SEQ ID NO: 26 | IgG1 Fc amino acid sequence comprising |
| | IgG1 hinge amino acid sequence |
| SEQ ID NO: 27 | IgG1 Fc amino acid sequence without hinge |
| | sequence |
| SEQ ID NO: 28 | IgG1 Fc amino acid sequence with protA |
| | mutation and without hinge sequence |
| SEQ ID NO: 29 | IgG1 Fc amino acid sequence with T366Y |
| | knob mutation (relative to SEQ ID NO: 13) |
| | and without hinge sequence |
| SEQ ID NO: 30 | IgG1 Fc amino acid sequence with T366Y |
| | knob mutation (relative to SEQ ID NO: 13) |
| | with protA mutation and without hinge |
| | sequence |
| SEQ ID NO: 31 | IgG1 Fc amino acid sequence with Y407T |
| | hole mutation (relative to SEQ ID NO: 13) and |
| | without hinge sequence |
| SEQ ID NO: 32 | IgG1 Fc amino acid sequence with Y407T |
| | hole mutation (relative to SEQ ID NO: 13) |
| | with protA mutation and without hinge |
| | sequence |
| SEQ ID NO: 33 | Human FVII active site mutant amino acid |
| | sequence |
| SEQ ID NO: 34 | Human FVII active site mutant amino acid |
| | sequence with mutations S344A and A341K |
| | relative to SEQ ID NO: 33 |
| SEQ ID NO: 35 | Nucleic acid sequence of pbudcd4.1 ICON |
| | Fc/knob and Fe/hole (construct JT075) |
| SEQ ID NO: 36 | 5′ Vector of the construct of SEQ ID NO: 35 |
| SEQ ID NO: 37 | 3′ Vector of the construct of SEQ ID NO: 35 |
| SEQ ID NO: 38 | Insert of the construct of SEQ ID NO: 35 |
| SEQ ID NO: 39 | Translated insert of SEQ ID NO: 38 |
| SEQ ID NO: 40 | Nucleic acid sequence of pVITRO2 ICON |
| | Fc/knob and Fc/hole (construct JT077) |
| SEQ ID NO: 41 | 5′ Vector of the construct of SEQ ID NO: 40 |
| SEQ ID NO: 42 | 3′ Vector of the construct of SEQ ID NO: 40 |
| SEQ ID NO: 43 | Insert of the construct of SEQ ID NO: 40 |
| SEQ ID NO: 44 | Translated insert of SEQ ID NO: 43 |
| SEQ ID NO: 45 | Nucleic acid sequence of pSELECT-neo |
| | Fc/hole (construct JT090) |
| SEQ ID NO: 46 | 5′ Vector of the construct of SEQ ID NO: 45 |
| SEQ ID NO: 47 | 3′ Vector of the construct of SEQ ID NO: 45 |
| SEQ ID NO: 48 | Insert of the construct of SEQ ID NO: 45 |
| SEQ ID NO: 49 | Translated insert of SEQ ID NO: 48 |
| SEQ ID NO: 50 | Nucleic acid sequence of pSELECT-puro |
| | Fc/hole (construct JT091) |
| SEQ ID NO: 51 | 5′ Vector of the construct of SEQ ID NO: 50 |
| SEQ ID NO: 52 | 3′ Vector of the construct of SEQ ID NO: 50 |
| SEQ ID NO: 53 | Insert of the construct of SEQ ID NO: 50 |
| SEQ ID NO: 54 | Translated insert of SEQ ID NO: 53 |
| SEQ ID NO: 55 | Nucleic acid sequence of pSELECT-puro |
| | FVII-Fc/hole (construct JT092) |
| SEQ ID NO: 56 | 5′ Vector of the construct of SEQ ID NO: 55 |
| SEQ ID NO: 57 | 3′ Vector of the construct of SEQ ID NO: 55 |
| SEQ ID NO: 58 | Insert of the construct of SEQ ID NO: 55 |
| SEQ ID NO: 59 | Translated insert of SEQ ID NO: 58 |

### Immunoconjugate Production

In some embodiments, methods of producing the immunoconjugate include expression in mammalian cells such as BHK cells. In further embodiments, cell lines may include HEK 293, CHO, and SP2/0. Two-armed FVII-Fc immunoconjugates may be generated by mammalian expression of the expression constructs described in Table 2A. One-armed FVII-Fc immunoconjugates are generated by co-expression of the expression constructs listed in Table 2B. A two-step purification process may be utilized to purify the one-armed molecules from cell culture supernatants, which may also contain two-armed molecules and unarmed Fc molecules. In some embodiments, the one-armed or two-armed FVII-Fc immunoconjugates are produced as fusion proteins (FVII-Fc) or produced as chemical conjugates.

In some embodiments, the production of the immunoconjugates is by way of transient expression from viral-vectors infecting host cells. In other embodiments, production of the immunoconjugates is by way of expression of stably-transformed expression vectors in the host cell genome.

In some embodiments, one or more of the sequences of SEQ ID NOs:35-59 may be utilized to produce the ICON-1.5 immunoconjugate.

In some embodiments, the production of one-armed immunoconjugates results in a composition substantially free of two-armed immunoconjugates. In some embodiments, the production of two-armed immunoconjugates results in a composition substantially free of one-armed immunoconjugates. In some embodiments, compositions or formulations comprising the one-armed immunoconjugates are substantially free of two-armed immunoconjugates. In some embodiments, compositions or formulations comprising the two-armed immunoconjugates are substantially free of one-armed immunoconjugates.

Expression of the one-armed immunoconjugate comprises the utilization of two open reading frames. The first open reading frame (FVII-Fc expression construct) encodes a mutated FVII sequence, win frame with or without a linker region, with or without the human IgG1 hinge sequence, and the human IgG1 Fc region (SEQ ID NO:27). The second open reading frame (Fc-only expression construct) encodes the human IgG1 hinge and Fc sequence (SEQ ID NO:26). For transient transfections, expression of the open reading frames is accomplished with mammalian promoter sequences that enable expression in mammalian expression hosts such as BHK21, CHO-S, and HEK293 cells.

The one-armed immunoconjugate may be generated by transient transfection of ExpiCHO or Expi293 cells using the corresponding vendor's transfection protocols (Catalog number A29133 and A14635; ThermoFisher Scientific, Waltham, Mass.). Cells at the manufacturer's recommended cell density and viability are transfected with 1 microgram of DNA per mL of culture with ⅓ of the DNA (i.e. 0.33 micrograms/mL) being the FVII-Fc expression construct, and two thirds of the DNA (i.e. 0.66 micrograms/mL) the Fc-only expression construct. One day post-transfection, the cells are fed with the appropriate reagents. Once viability is between 70% and 80%, the cell culture supernatant is collected by centrifugation and depth filtration. Immunoconjugates are isolated from the cleared supernatant with a protein A capture step using MabSelect SuRe resin (GE Healthcare Bio-Sciences, Pittsburgh, Pa.). One-armed immunoconjugates are subsequently isolated from the neutralized affinity eluate by size exclusion chromatography or anion exchange chromatography using appropriate resins (GE Healthcare Bio-Sciences. Pittsburgh, Pa.). The size exclusion and/or anion exchange chromatography enables removal of protein aggregates, two-armed immunoconjugates, and Fc-only homodimers when present in the affinity eluate.

In some embodiments, the immunoconjugate is post-translationally modified. Post-translational modification includes: myristoylation, glypiation, palmitoylation, prenylation, lipoylation, acylation, alkylation, butrylation, gamma-carboxylation, glycosylation (N-glycosylation, O-glycosylation, fucosylation, and mannosylation), propionylation, succinylation, and sulfation.

In one embodiment the immunoconjugates are stably expressed. In one embodiment, the immunoconjugates are transiently expressed

TABLE 2A
Immunoconjugate
Exemplary Two-Armed Immunoconjugates

| | Expression | Component | Component | |
|---|---|---|---|---|
| Immunoconjugate No. | Cassette No. | No. | description | Component sequence ID |
| Immunoconjugate No. 1 | 1 | 1 | FVII | SEQ ID 12 |
| | | 2 | Fc | SEQ ID 13 |
| Immunoconjugate No. 2 | 2 | 1 | FVII | SEQ ID 12 |
| | | 2 | Linker (with upper | SEQ ID 8, 9, 10, 11, or |
| | | | hinge) | GSA |
| | | 3 | Fc | SEQ ID 13 |
| Immunoconjugate No. 3 | 1 | 1 | FVII | SEQ ID 34 |
| | | 2 | Fc and hinge | SEQ ID 27 and 25 |
| Immunoconjugate No. 4 | 2 | 1 | FVII | SEQ ID 33 |
| | | 2 | Linker | GSA |
| | | 3 | Fc and hinge | SEQ ID 27 and 23 |

TABLE 2B
Exemplary One-Armed Immunoconjugates

| Immunoconjugate | Expression | Component | | |
|---|---|---|---|---|
| No. | Cassette No. | No. | Component description | Component sequence ID |
| Immunoconjugate No. 1 | 1 | 1 | FVII | SEQ ID 12 |
| | | 2 | Fc | SEQ ID 13 |
| | 2 | 1 | Fc | SEQ ID 13 |
| Immunoconjugate No. 2 | 1 | 1 | FVII | SEQ ID 12 |
| | | 2 | Linker (with upper hinge) | SEQ ID 8, 9, 10, 11, |
| | | | | or GSA |
| | | 3 | Fc | SEQ ID 13 |
| | 2 | 1 | Fc | SEQ ID 13 |
| Immunoconjugate No. 3 | 1 | 1 | FVII | SEQ ID 12 |
| | | 2 | Fc with T366Y knob | SEQ ID 15 |
| | 2 | 1 | Fc with Y407T hole | SEQ ID 17 |
| Immunoconjugate No. 4 | 1 | 1 | FVII | SEQ ID 12 |
| | | 2 | Linker (with upper hinge) | SEQ ID 8, 9, 10, 11, |
| | | | | or GSA |
| | | 3 | Fc with T366Y knob | SEQ ID 15 |
| | 2 | 1 | Fc with Y407T hole | SEQ ID 17 |
| Immunoconjugate No. 5 | 1 | 1 | FVII | SEQ ID 12 |
| | | 2 | Fc with Y407T hole | SEQ ID 17 |
| | 2 | 1 | Fc with T366Y knob | SEQ ID 15 |
| Immunoconjugate No. 6 | 1 | 1 | FVII | SEQ ID 10 |
| | | 2 | Linker (with upper hinge) | SEQ ID 8, 9, 10, 11, |
| | | | | or GSA |
| | | 3 | Fc with Y407T hole | SEQ ID 17 |
| | 2 | 1 | Fc with T366Y knob | SEQ ID 15 |
| Immunoconjugate No. 7 | 1 | 1 | FVII | SEQ ID 34 |
| | | 2 | Fc and hinge | SEQ ID 28 and 22 |
| | 2 | 1 | Fc and hinge | SEQ ID 28 and 22 |
| Immunoconjugate No. 8 | 1 | 1 | FVII | SEQ ID 34 |
| | | 2 | Linker | GSA |
| | | 3 | Fc and hinge | SEQ ID 27 and 22 |
| | 2 | 1 | Fc and hinge | SEQ ID 27 and 22 |
| Immunoconjugate No. 9 | 1 | 1 | FVII | SEQ ID 33 |
| | | 2 | Fc (with T366Y knob) | SEQ ID 29 and 22 |
| | | | and hinge | |
| | 2 | 1 | Fc (with Y407T hole) and | SEQ ID 31 and 22 |
| | | | hinge | |
| Immunoconjugate No. | 1 | 1 | FVII | SEQ ID 34 |
| 10 | | 2 | Linker | GGG |
| | | 3 | Fc (with T366Y knob) | SEQ ID 30 and 22 |
| | | | and hinge | |
| | 2 | 1 | Fc (with Y407T hole) and | SEQ ID 32 and 22 |
| | | | hinge | |
| Immunoconjugate No. | 1 | 1 | FVII | SEQ ID 34 |
| 11 | | 2 | Fc (with Y407T hole) and | SEQ ID 30 and 22 |
| | | | hinge | |
| | 2 | 1 | Fc (with T366Y knob) | SEQ ID 32 and 22 |
| | | | and hinge | |
| Immunoconjugate No. | 1 | 1 | FVII | SEQ ID 34 |
| 12 | | 2 | Linker | GSA |
| | | 3 | Fc (with Y407T hole) and | SEQ ID 29 and 22 |
| | | | hinge | |
| | 2 | 1 | Fc (with T366Y knob) | SEQ ID 31 and 22 |
| | | | and hinge | |

### Arming the Immunoconjugate

In some embodiments, radionuclides, radioisotopes, or other entities such as toxins are coupled to an immunoconjugate of the present disclosure. In some embodiments, bifunctional chelators are utilized to link a radionuclide to the immunoconjugate. In one embodiment, the chelator is first attached to the immunoconjugate, and the chelator-immunoconjugate is contacted with a metallic radioisotope, thus arriving at an immunoconjugate further conjugated to radionuclide or radioisotope. Methods of conjugating radionuclides to proteins are disclosed in U.S. Pat. Nos. 4,824,659, 5,574,140, and U.S. Patent Publication US20040136908A1.

In some embodiments, the conjugation of the molecules to the immunoconjugates of the present disclosure may be done via lysine or cysteine residues of the Fc or FVII regions. In one embodiment, an arming molecule is conjugated to the immunoconjugate at a lysine or cysteine residue of the immunoconjugate's Fc region. In one embodiment, the conjugation occurs at only one of the Fc monomers of the Fc dimer. In one embodiment, the conjugation occurs at both of the Fc monomers of the Fc dimer, resulting in multiple arming molecules conjugated to the immunoconjugate. In one embodiment, the arming molecule is conjugated to the immunoconjugate's FVII at a lysine or cysteine residue.

In some embodiments, bifunctional chelators include the following: dithylenetriamine pentaacetic acid (DTPA) series of amino acids, hydroxamic acid-based bifunctional chelating agents, p-SCN-Bz-HEHA (1,4,7,10,13,16-hexaazacyclo-octadecane-N,N′,N,N′-hexaacetic acid, 1,4,7,10-tetraazacyclododecane N,N′,N″,N′″-tetraacetic acid (DOTA), NOTA, TETA, diethylenetriaminepentaacetic acid (DTPA), monomethyl or cyclohexyl analogs of 2-benzyl-DTPA, ethylenediaminetetraacetic acid (EDTA), macrocyclic polyethers, porphyrins, polyamines, crown ethers, polyoximes, and thiosemicarbazones.

In some embodiments, the immunoconjugate is conjugated to a cytotoxic agent. The term “cytotoxic agent” as used herein refers to a substance that inhibits or prevents the function of cells and/or causes destruction of cells. The term is intended to include radioactive isotopes (e.g., ²¹¹At, ¹³¹I, ¹²⁵I, ⁹⁰Y, ¹⁸⁶Re, ¹⁸⁸Re, ¹⁵³Sm, ²¹²Bi, ³²P, αCo, and radioactive isotopes of Lu), chemotherapeutic agents, alkylating agents, and toxins such as small molecule toxins or enzymatically active toxins of bacterial, fungal, plant, or animal origin; including synthetic analogs and derivatives thereof.

In some embodiments, therapeutic agents such as cytotoxic agents, anti-angiogenic agents, pro-apoptotic agents, hormones, hormone antagonists, chemokines, drugs, prodrugs, toxins, enzymes, or other agents may be used, conjugated to the immunoconjugate of the present disclosure Drugs of use may possess a pharmaceutical property selected from the group consisting of antimitotic, antikinase, alkylating, antimetabolite, antibiotic, alkaloid, anti-angiogenic, pro-apoptotic agents, and combinations thereof.

In some embodiments, photosensitizers may be conjugated to the immunoconjugates of the present disclosure. In some embodiments, photosensitizers may be selected from photodynamic dyes (those capable of destroying target tissue), hematoporphyrins such as dihematoporphyrin ethers and dimers and trimers thereof, aminolevulinic acids, porphyrins such as boronated porphyrins and benzoporphyrins, merocyanines, porphycenes, porfimer sodium, verteporfin, VYSUDINE, CIBA VISION, PHOTOFRIN II, PH-10, chlorins, zinc phthalocyanine, purpurins, pheophorbides, SnCe6, and monoclonal antibody-dye conjugates. See U.S. Pat. Nos. 6,693,093, 6,443,976, 4,968,715, 5,190,966, 5,028,621, 4,866,168, 4,649,151, 5,438,071, 5,079,262, 4,883,790, 4,920,143, 5,095,030, 5,171,749, 5,171,741, 5,173,504, 5,166,197, 5,198,460, 5,002,962, and 5,093,349; all incorporated by reference herein. In some embodiments, targeted photodynamic therapy (PDT) is utilized to activate the conjugated photosensitizers, thus weakening/lysing the cells in closest proximity. In one embodiment, the PDT activates the photosensitizer with a non-thermal laser.

In some embodiments, drugs of use may include 5-fluorouracil, aplidin, azaribine, anastrozole, anthracyclines, bendamustine, bleomycin, bortezomib, bryostatin-1, busulfan, calicheamycin, camptothecin, carboplatin, 10-hydroxycamptothecin, carmustine, celebrex, chlorambucil, cisplatin (CDDP), Cox-2 inhibitors, irinotecan (CPT-11), SN-38, carboplatin, cladribine, camptothecans, cyclophosphamide, cytarabine, dacarbazine, docetaxel, dactinomycin, daunorubicin, doxorubicin, 2-pyrrolinodoxorubicine (2P-DOX), cyano-morpholino doxorubicin, doxorubicin glucuronide, epirubicin glucuronide, estramustine, epipodophyllotoxin, estrogen receptor binding agents, etoposide (VP16), etoposide glucuronide, etoposide phosphate, floxuridine (FUdR), 3′,5′-O-dioleoyl-FudR, (FUdR-dO), fludarabine, flutamide, farnesyl-protein transferase inhibitors, gemcitabine, hydroxyurea, idarubicin, ifosfamide. L-asparaginase, lenolidamide, leucovorin, lomustine, mechlorethamine, melphalan, mercaptopurine, 6-mercaptopurine, methotrexate, mitoxantrone, mithramycin, mitomycin, mitotane, navelbine, nitrosourea, plicomycin, procarbazine, paclitaxel, pentostatin. PSI-341, raloxifene, semustine, streptozocin, tamoxifen, taxol, temazolomide (an aqueous form of DTIC), transplatinum, thalidomide, thioguanine, thiotepa, teniposide, topotecan, uracil mustard, vinorelbine, vminblastine, vincristine, vmca alkaloids, maytansinoids, maytansinoid analogs, benzodiazepine, taxoid, CC-1065, CC1065 analog, duocarmycin, duocarmycin analog, calicheamicin, dolastatin, dolastatin analog, auristatin, tomaymycin derivative, and a leptomycin derivative.

In some embodiments, toxins of use may include ricin, abrin, alpha toxin, saporin, ribonuclease (RNase), e.g., onconase, DNase 1, Staphylococcal enterotoxin-A, pokeweed antiviral protein, gelonin, diphtheria toxin, Pseudomonas exotoxin, and Pseudomonas endotoxin.

In some embodiments, chemokines of use may include RANTES, MCAF, MIP1-alpha. MIP1-Beta and IP-10.

In some embodiments, anti-angiogenic agents, such as angiostatin, baculostatin, canstatin, maspin, anti-VEGF antibodies, anti-P1GF peptides and antibodies, anti-vascular growth factor antibodies, anti-Flk-1 antibodies, anti-Flt-1 antibodies and peptides, anti-Kras antibodies, anti-cMET antibodies, anti-MIF (macrophage migration-inhibitory factor) antibodies, laminin peptides, fibronectin peptides, plasminogen activator inhibitors, tissue metalloproteinase inhibitors, interferons, interleukin-12, IP-10, Gro-B, thrombospondin, 2-methoxyoestradiol, proliferin-related protein, carboxiamidotriazole, CMI01, Marimastat, pentosan polysulphate, angiopoietin-2, interferon-alpha, herbimycin A, PNU 45156E, 16K prolactin fragment, Linomide (roquinimex), thalidomide, pentoxifylline, genistein, TNP-470, endostatin, paclitaxel, accutin, angiostatin, cidofovir, vincristine, bleomycin, AGM-1470, platelet factor 4 or minocycline may be of use.

In some embodiments, immunomodulators of use may be selected from a cytokine, a stem cell growth factor, a lymphotoxin, a hematopoietic factor, a colony stimulating factor (CSF), an interferon (IFN), erythropoietin, thrombopoietin and a combination thereof. Specifically useful are lymphotoxins such as tumor necrosis factor (TNF), hematopoietic factors, such as interleukin (IL), colony stimulating factor, such as granulocyte-colony stimulating factor (G-CSF) or granulocyte macrophage-colony stimulating factor (GM-CSF), interferon, such as interferons-α, β or γ, and stem cell growth factor, such as that designated “Si factor”. Included among the cytokines are growth hormones such as human growth hormone, N-methionyl human growth hormone, and bovine growth hormone; parathyroid hormone; thyroxine; insulin; proinsulin, relaxin; prorelaxin; glycoprotein hormones such as follicle stimulating hormone (FSH), thyroid stimulating hormone (TSH), and luteinizing hormone (LH), hepatic growth factor: prostaglandin, fibroblast growth factor, prolactin; placental lactogen, OB protein, tumor necrosis factor-α and -β, mullerian-inhibiting substance: mouse gonadotropin-associated peptide; inhibin: activin; vascular endothelial growth factor; integrin; thrombopoietin (TPO); nerve growth factors such as NGF-1: platelet-growth factor; transforming growth factors (TGFs) such as TGF-α and TGF-β; insulin-like growth factor-I and -II: erythropoietin (EPO); osteoinductive factors; interferons such as interferon-α, -β, and -γ; colony stimulating factors (CSFs) such as macrophage-CSF (M-CSF); interleukins (ILs) such as IL-1, IL-la, IL-2, IL-3. IL-4, IL-5, IL-6. IL-7, IL-8, IL-9, IL-10, IL-11, IL-12; IL-13, IL-14. IL-15, IL-16, IL-17, IL-18, IL-21, IL-25, LIF, kit-ligand or FLT-3, angiostatin, thrombospondin, endostatin, tumor necrosis factor, and LT.

In some embodiments, immunoconjugates of the present disclosure are co-administered with IL-15.

In some embodiments, radionuclides or radioisotopes of use include, but are not limited to a gamma-emitter, a beta-emitter, an alpha-emitter, a positron-emitter, and combinations of two or more thereof. In some embodiments radionuclides of use include, but are not limited to ¹¹¹In, ¹⁷⁷Lu, ¹³⁷Ba, ²¹²Bi, ²¹³Bi, ²¹¹At, ⁶⁰Co, ⁶²Cu, ⁶⁷Cu, ¹³⁷Cs, ⁹⁰Y, ¹²⁵I, ¹³¹I, ³²P, ³³P, ⁴⁷Sc, ¹¹¹Ag, ⁶⁷Ga, ¹⁴²Pr, ¹⁵³Sm, ¹⁶¹Tb, ¹⁶⁶Dy, ¹⁶⁶Ho, ¹⁸⁶Re, ¹⁸⁸Re, ¹⁸⁹Re, ²¹²Pb, ²²³Ra, ²²⁵Ac, ⁵⁹Fe, ⁷⁵Se, ⁷⁷As, ⁸⁹Sr, ⁹⁹Mo, ¹⁰⁵Rh, ¹⁰⁹Pd, ¹⁴³R, ¹⁴⁹Pm, ¹⁶⁹Er, ¹⁹⁴Ir, ¹⁹⁹Au, and ²¹¹Pb.

In some embodiments, the radionuclides preferably have a decay-energy in the range of 20 to 6,000 keV, preferably in the ranges 60 to 200 keV for an Auger emitter, 100-2,500 keV for a beta emitter, 20-4,000 keV for a gamma emitter, and 4,000-6,000 keV for an alpha emitter. Maximum decay energies of useful beta-particle-emitting nuclides are preferably 20-5,000 keV, more preferably 100-4,000 keV, and most preferably 500-2,500 keV. Also preferred are radionuclides that substantially decay with Auger-emitting particles. For example, Co-58, Ga-67, Br-80 μm, Tc-99 μm, Rh-103 μm, Pt-109, In-111, Sb-119, I-125, Ho-161, Os-189m and Ir-192. Decay energies of useful beta-particle-emitting nuclides are preferably <1,000 keV, more preferably <100 keV, and most preferably <70 keV. Also preferred are radionuclides that substantially decay with generation of alpha-particles. Such radionuclides include, but are not limited to: Dy-152, At-211, Bi-212, Ra-223, Rn-219, Po-215, Bi-211, Ac-225, Fr-221, At-217, Bi-213 and Fm-255. Decay energies of useful alpha-particle-emitting radionuclides are preferably 2,000-10,000 keV, more preferably 3,000-8,000 keV, and most preferably 4,000-7,000 keV.

In some embodiments, additional radionuclides of use include ¹¹C, ¹³N, ¹⁵O, ⁷⁵Br, ¹⁹⁸Au, ²²⁴Ac, ¹²⁶I, ¹³³I, ⁷⁷Br, ¹¹³In, ⁹⁵Ru, ⁹⁷Ru, ¹⁰⁵Ru, ¹⁰⁷Hg, ²⁰³Hg, ^{121m}Te, ^{122m}Te, ^{125m}Te, ¹⁶⁵Tm, ¹⁶⁷Tm, ¹⁶⁸Tm, ¹⁹⁷Pt, ¹⁰⁹Pd, ¹⁰⁵Rh, ¹⁴²Pr, ¹⁴³Pr, ¹⁶¹Tb, ¹⁶⁶Ho, ¹⁹⁹Au, ⁵⁷Co, ⁵⁸Co, ⁵¹Cr, ⁵⁹Fe, ⁷⁵Se, ²⁰¹Tl, ²²⁵Ac, ⁷⁶Br, ¹⁶⁹Yb, and the like. Some useful diagnostic radionuclides include ¹⁸F, ⁵²Fe, ⁶²Cu, ⁶⁴Cu, ₆₇Cu, ⁶⁷Ga, ⁶⁸Ga, ⁸⁶Y, ⁸⁹Zr, ⁹⁴Tc, ^{94m}Tc, ^{99m}Tc, or ¹¹¹In.

In some embodiments, therapeutic agents of use may include a photoactive agent or dye. Fluorescent compositions, such as fluorochrome, and other chromogens, or dyes, such as porphyrins sensitive to visible light, have been used to detect and to treat lesions by directing the suitable light to the lesion. In therapy, this has been termed photoradiation, phototherapy, or photodynamic therapy. See Jon et al. (eds.), Photodynamic Therapy of Tumors and Other Diseases (Libreria Progetto 1985); van den Bergh, Chem. Britain (1986), 22:430 Moreover, monoclonal antibodies have been coupled with photoactivated dyes for achieving phototherapy. See Mew et al., J. Immunol. (1983), 130:1473; idem., Cancer Res (1985), 45:4380; Oserotff et al., Proc Natl. Acad. Sci. USA (1986), 83:8744; idem., Photochem. Photobiol. (1987), 46:83; Hasan et al., Prog Clin. Biol. Res. (1989), 288:471; Tatsuta et al., Lasers Surg. Med. (1989), 9:422; Pelegrin et al., Cancer (1991), 67:2529.

In some embodiments, other therapeutic agents of use may comprise oligonucleotides, such as antisense oligonucleotides that are directed against oncogenes and oncogene products, such as bcl-2 or p53. In one embodiment, a therapeutic oligonucleotide is an siRNA.

In some embodiments, therapeutic agents such as cytotoxic agents, anti-angiogenic agents, pro-apoptotic agents, hormones, hormone antagonists, chemokines, drugs, prodrugs, toxins, enzymes, or other agents may be linked or conjugated to the immunoconjugate of the present disclosure by a linker. Linkers disclosed herein are described in U.S. Patent Publication Nos.: US2005/0169933, US2009/0274713, and in WO/2009/0134976.

In one embodiment, a linker may be selected from the group of a cleavable linker, a non-cleavable linker, a hydrophilic linker, and a dicarboxylic acid based linker. Suitable linkers are known in the art, and may include, for example, disulfide groups, thioether groups, acid labile groups, photolabile groups, peptidase labile groups, and esterase labile groups. Linkers may also include charged linkers, and hydrophilic forms thereof.

In one embodiment, a linker may be selected from the group consisting of N-succinimidyl 4-(2-pyridyldithio)pentanoate (SPP) or N-succinimidyl 4-(2-pyridyldithio)-2-sulfopentanoate (sulfo-SPP); N-succinimidyl 4-(2-pyridyldithio)butanoate (SPDB) or N-succinimidyl 4-(2-pyridyldithio)-2-sulfobutanoate (sulfo-SPDB); N-succinimidyl 4-(maleimidomethyl)cyclohexanecarboxylate (SMCC); N-sulfosuccinimidyl 4-(maleimidomethyl)cyclohexanecarboxylate (sulfoSMCC); N-succinimidyl-4-(iodoacetyl)-aminobenzoate (SIAB); and N-succinimidyl-[(N-maleimidopropionamido)-tetraethyleneglycol]ester (NHS-PEG4-maleimide). In a certain embodiment, the linker is N-succinimidyl-[(N-maleimidopropionamido)-tetraethyleneglycol]ester (NHS-PEG4-maleimide).

### METHODS OF THE INVENTION

Provided herein are methods for using the immunoconjugate dimers described herein, for treating a patient having a disease or disorder associated with neovascularization (e.g. tumor-associated neovascularization, such as cancer). The methods provided herein comprise administering to the patient a therapeutically effective amount of one or more immunoconjugate dimers provided herein for the treatment of a disease associated with pathological neovascularization including, but not limited to atherosclerosis, rheumatoid arthritis, endometriosisocular melanoma, solid tumor, primary or metastatic solid tumors (including but not limited to melanoma, renal, prostate, breast, ovarian, brain, neuroblastoma, head and neck, pancreatic, bladder, endometrial and lung cancer), diabetic macular edema (DME), macular edema following retinal vein occlusion (RVO), proliferative diabetic retinopathy, wet age-related macular degeneration (AMD), retinopathy of prematurity (ROP), and neovascular glaucoma. The immunoconjugates provided herein are amenable for use in any disease or disorder in which neovascularization is implicated. In one embodiment, methods for treating a patient for any cancer are provided. In one embodiment, methods for treating ocular melanoma are provided.

As used herein, the term “patient” includes both humans and other species, including other mammal species. The invention thus has both medical and veterinary applications. In veterinary compositions and treatments, immunoconjugates are constructed using targeting and effector domains derived from the corresponding species.

In one aspect, an immunoconjugate dimer provided herein is administered to the eye of a patient in need of treatment of ocular melanoma. In one embodiment, the treatment comprises multiple dosing sessions of the immunoconjugate dimer. In one embodiment, a monomer unit of the immunoconjugate has the amino acid sequence of SEQ ID NO:4 or 5. In a specific embodiment, a monomer unit of the immunoconjugate dimer has the amino acid sequence of SEQ ID NO:4. In another specific embodiment, a monomer unit of the immunoconjugate dimer has the amino acid sequence of SEQ ID NO:5. In another specific embodiment, a monomer unit of the immunoconjugate dimer comprises the targeting domain of SEQ ID NO:1, SEQ ID NO:2, SEQ ID NO:12, SEQ ID NO:33, and SEQ ID NO:34. In one aspect, the immunoconjugate dimer provided herein is administered to treat a metastasis of ocular melanoma. Such metastasis includes metastatic events that occur distal to the eye, i.e., liver, lung, bone, skin, brain, lymph nodes, and adrenal tissues.

In one aspect, an immunoconjugate dimer provided herein is administered to the eye of a patient in need of treatment of wet AMD. In one embodiment, the treatment comprises multiple dosing sessions of the immunoconjugate dimer. In one embodiment, a monomer unit of the immunoconjugate dimer has the amino acid sequence of SEQ ID NO:4 or 5. In a specific embodiment, a monomer unit of the immunoconjugate dimer has the amino acid sequence of SEQ ID NO:4. In another specific embodiment, a monomer unit of the immunoconjugate dimer has the amino acid sequence of SEQ ID NO:5. In another specific embodiment, a monomer unit of the immunoconjugate dimer comprises the targeting domain of SEQ ID NO:1, SEQ ID NO:2, SEQ ID NO:12, SEQ ID NO:33, or SEQ ID NO:34, and comprises an effector domain (inclusive of a hinge region) of SEQ ID NO:13, SEQ ID NO:14, SEQ ID NO:15, SEQ ID NO:16, SEQ ID NO:17, SEQ ID NO:18, and/or SEQ ID NO:26.

In one embodiment, the method of treating wet AMD comprises preventing, inhibiting or reversing choroidal neovascularization in the eye of the patient in need of treatment. In a further embodiment, choroidal neovascularization is reversed by at least about 10%, at least about 20%, at least about 30% or at least about 40% after treatment, as compared to the choroidal neovascularization that was present in the afflicted eye of the patient prior to treatment.

Other ocular disorders associated with ocular neovascularization are treatable with the immunoconjugates and methods provided herein. The ocular neovascularization, in one embodiment, is choroidal neovascularization. In another embodiment the ocular neovascularization is retinal neovascularization. In yet another embodiment, the ocular neovascularization is corneal neovascularization. In yet another embodiment, the ocular neovascularization is an tumor-associated neovascularization of the eye. Accordingly, in one embodiment, an ocular disorder associated with choroidal, retinal or corneal neovascularization is treatable by one or more of the methods provided herein. In a further embodiment, the method comprises administering to the eye of a patient in need thereof, one of the immunoconjugate dimers described herein. In a further embodiment, the treatment comprises multiple dosing sessions of the immunoconjugate dimer. In one embodiment, a monomer unit of the immunoconjugate dimer has the amino acid sequence of SEQ ID NO:4 or 5. In a specific embodiment, a monomer unit of the immunoconjugate dimer has the amino acid sequence of SEQ ID NO:4. In another specific embodiment, a monomer unit of the immunoconjugate dimer has the amino acid sequence of SEQ ID NO:5. In another specific embodiment, a monomer unit of the immunoconjugate dimer comprises the targeting domain of SEQ ID NO:1 SEQ ID NO:12, SEQ ID NO:33, or SEQ ID NO:34 and comprises effector domain (inclusive of a hinge region) of SEQ ID NO:13, SEQ ID NO:14, SEQ ID NO:15, SEQ ID NO:16, SEQ ID NO:17, SEQ ID NO:18, and/or SEQ ID NO:26. In yet a further embodiment, the immunoconjugate comprises the mutated FVII domain of SEQ ID NO:2 conjugated to a human IgG1 region of SEQ ID NO:13-18. Conjugation, in one embodiment is via an IgG1 hinge region, e.g., via the sequence of SEQ ID NO:8-11 and 19-25. In another embodiment, conjugation is via the GSA, GGG, or GGSS (SEQ ID NO:11) linker, or repeats thereof.

For example, in one embodiment, a patient in need of treatment of proliferative diabetic retinopathy, wet age-related macular degeneration (AMD), retinopathy of prematurity (ROP), or neovascular glaucoma is treated with one of the immunoconjugates provided herein, for example, via intravitreal injection, suprachoroidal injection or topical administration (e.g., via eyedrops) of the immunoconjugate into the affected eye. Treatment in one embodiment occurs over multiple dosing sessions. With respect to the aforementioned disorders, ocular neovascularization is said to be “associated with” or “secondary to” the respective disorder.

In one embodiment, a patient in need of treatment of macular edema following retinal vein occlusion (RVO) is treated by one of the immunoconjugate dimers provided herein. In one embodiment, the treatment comprises multiple dosing sessions of the immunoconjugate dimer. In one embodiment, a monomer unit of the immunoconjugate dimer has the amino acid sequence of SEQ ID NO:4 or 5. In a specific embodiment, a monomer unit of the immunoconjugate dimer has the amino acid sequence of SEQ ID NO:4. In another specific embodiment, a monomer unit of the immunoconjugate dimer has the amino acid sequence of SEQ ID NO:5. In another specific embodiment, a monomer unit of the immunoconjugate dimer comprises the targeting domain of SEQ ID NOs:1, 2, 12, 33, or 34 and comprises an effector domain (inclusive of a hinge region) of SEQ ID NO:13, SEQ ID NO:14, SEQ ID NO:15, SEQ ID NO:16, SEQ ID NO:17, SEQ ID NO:18, and/or SEQ ID NO:26. In one embodiment, the immunoconjugate is administered to the patient over multiple dosing sessions, for example, via intravitreal administration at each dosing session.

In another embodiment, a patient in need of treatment of diabetic macular edema (DME) is treated by one of the immunoconjugate dimers provided herein. In one embodiment, the treatment comprises multiple dosing sessions of the immunoconjugate dimer. In one embodiment, a monomer unit of the immunoconjugate dimer has the amino acid sequence of SEQ ID NO:4 or 5. In a specific embodiment, a monomer unit of the immunoconjugate dimer has the amino acid sequence of SEQ ID NO:4. In another specific embodiment, a monomer unit of the immunoconjugate dimer has the amino acid sequence of SEQ ID NO:5. In another specific embodiment, a monomer unit of the immunoconjugate dimer comprises the targeting domain of SEQ ID NOs:1, 2, 12, 33, or 34 and comprises an effector domain (inclusive of a hinge region) of SEQ ID NO:13, SEQ ID NO:14, SEQ ID NO:15, SEQ ID NO:16, SEQ ID NO:17, SEQ ID NO:18, and/or SEQ ID NO:26. In one embodiment, the immunoconjugate dimer is administered to the patient over multiple dosing sessions, for example, via intravitreal administration at each dosing session. In even a further embodiment, the immunoconjugate dimer is administered intravitreally at each dosing session.

In yet another embodiment, diabetic retinopathy is treated via one of the immunoconjugates provided herein, in a patient in need thereof, for example, a patient with DME. In one embodiment, the treatment comprises multiple dosing sessions of the immunoconjugate dimer. In one embodiment, a monomer unit of the immunoconjugate dimer has the amino acid sequence of SEQ ID NO:4 or 5. In a specific embodiment, a monomer unit of the immunoconjugate dimer has the amino acid sequence of SEQ ID NO:4. In another specific embodiment, a monomer unit of the immunoconjugate dimer has the amino acid sequence of SEQ ID NO:5. In another specific embodiment, a monomer unit of the immunoconjugate dimer comprises the targeting domain of SEQ ID NOs: 1, 2, 12, 33, or 34 and comprises an effector domain (inclusive of a hinge region) of SEQ ID NO:13, SEQ ID NO:14, SEQ ID NO:15, SEQ ID NO:16, SEQ ID NO:17, SEQ ID NO:18, and/or SEQ ID NO:26. In one embodiment, the immunoconjugate dimer is administered to the patient over multiple dosing sessions, for example, via intravitreal administration at each dosing session. In even a further embodiment, the immunoconjugate dimer is administered to the patient over multiple dosing sessions, for example, via intravitreal administration at each dosing session.

In one embodiment described herein, one or more of the immunoconjugates provided herein is used in a method to treat a disease or disorder associated with tumor neovascularization in a patient in need thereof, for example, a cancer patient. In one embodiment, the method comprises administering to the patient, for example via intratumoral or intravenous injection, a composition comprising a therapeutically effective amount of an immunoconjugate dimer described herein. In one embodiment, the treatment comprises multiple dosing sessions of the immunoconjugate dimer. In one embodiment, a monomer unit of the immunoconjugate dimer has the amino acid sequence of SEQ ID NO:4 or 5. In a specific embodiment, a monomer unit of the immunoconjugate dimer has the amino acid sequence of SEQ ID NO:4. In another specific embodiment, a monomer unit of the immunoconjugate dimer has the amino acid sequence of SEQ ID NO:5. In another specific embodiment, a monomer unit of the immunoconjugate dimer comprises the targeting domain of SEQ ID NOs:1, 2, 12, 33, or 34 and comprises an effector domain (inclusive of a hinge region) of SEQ ID NO:13, SEQ ID NO:14, SEQ ID NO:15, SEQ ID NO:16, SEQ ID NO:17, SEQ ID NO:18, and/or SEQ ID NO:26.

In cancer treatments, the immunoconjugate dimer is used for treating a variety of cancers, particularly primary or metastatic solid tumors, including but not limited to melanoma, renal, prostate, breast, ovarian, brain, neuroblastoma, head and neck, pancreatic, bladder, endometrial and lung cancer. In one embodiment, the cancer is a gynecological cancer. In a further embodiment, the gynecological cancer is serous, clear cell, endometriod or undifferentiated ovarian cancer. The immunoconjugate dimer in one embodiment is employed to target the tumor vasculature, particularly vascular endothelial cells, and/or tumor cells. Without wishing to be bound by theory, targeting the tumor vasculature can offer several advantages for cancer immunotherapy with one or more of the immunoconjugate dimers described herein, as follows. (i) some of the vascular targets including tissue factor should be the same for all tumors; (ii) immunoconjugates targeted to the vasculature do not have to infiltrate a tumor mass in order to reach their targets; (iii) targeting the tumor vasculature should generate an amplified therapeutic response, because each blood vessel nourishes numerous tumor cells whose viability is dependent on the functional integrity of the vessel; and (iv) the vasculature is unlikely to develop resistance to an immunoconjugate, because that would require modification of the entire endothelium layer lining a vessel. Unlike previously described antiangiogenic methods that inhibit new vascular growth, immunoconjugate dimers provided herein elicit a cytolytic response to the neovasculature.

In another embodiment, one or more of the immunoconjugates described herein is used in a method for treating atherosclerosis or rheumatoid arthritis. In one embodiment, the treatment comprises multiple dosing sessions of the immunoconjugate dimer. In one embodiment, a monomer unit of the immunoconjugate dimer has the amino acid sequence of SEQ ID NO:4 or 5. In a specific embodiment, a monomer unit of the immunoconjugate dimer has the amino acid sequence of SEQ ID NO:4. In another specific embodiment, a monomer unit of the immunoconjugate dimer has the amino acid sequence of SEQ ID NO:5. In another specific embodiment, a monomer unit of the immunoconjugate dimer comprises the targeting domain of SEQ ID NOs:1, 2, 12, 33, or 34 and comprises an effector domain (inclusive of a hinge region) of SEQ ID NO:13, SEQ ID NO:14, SEQ ID NO:15, SEQ ID NO:16, SEQ ID NO:17, SEQ ID NO:18, and/or SEQ ID NO:26.

In one embodiment of a method for treating an ocular disorder such as ocular melanoma with an immunoconjugate dimer, for example, a method for treating wet AMD, diabetic retinopathy, diabetic macular edema, or choroidal neovascularization secondary to an ocular disorder such as wet AMD, the patient subjected to the treatment method substantially maintains his or her vision subsequent to the treatment (e.g., the single dosing session or multiple dosing sessions), as measured by losing fewer than 15 letters in a best-corrected visual acuity (BCVA) measurement, compared to the patient's BCVA measurement prior to undergoing treatment. In a further embodiment, the patient loses fewer than 10 letters, fewer than 8 letters, fewer than 6 letters or fewer than 5 letters in a BCVA measurement, compared to the patient's BCVA measurement prior to undergoing treatment.

In another embodiment of a method for treating an ocular disorder with an immunoconjugate dimer, for example, a method for treating ocular melanoma, wet AMD, diabetic retinopathy, diabetic macular edema, tumor-associated neovascularization, or choroidal neovascularization secondary to an ocular disorder such as wet AMD, the patient subjected to the treatment method substantially maintains his or her vision subsequent to the treatment (e.g., the single dosing session or multiple dosing sessions), as measured by gaining 15 or more letters in a best-corrected visual acuity (BCVA) measurement, compared to the patient's BCVA prior to the multiple dosing sessions. In a further embodiment, the patient gains about 15 letters or more, about 20 letters or more, about 25 letters or more in a BCVA measurement, compared to the patient's BCVA measurement prior to undergoing treatment. In even a further embodiment, the patient gains from about 15 to about 30 letters, from about 15 letters to about 25 letters or from about 15 letters to about 20 letters in a BCVA measurement, compared to the patient's BCVA measurement prior to undergoing treatment.

In one embodiment of a method for treating an ocular disorder in the eye of a patient in need thereof with an immunoconjugate dimer, for example, a method for treating wet AMD, diabetic retinopathy, diabetic macular edema, or choroidal neovascularization secondary to an ocular disorder such as wet AMD provided herein, the ocular neovascularization area, e.g., the choroidal neovascularization area of the eye of the patient is reduced in the eye of the patient, as compared to the ocular neovascularization area (e.g., CNV area) prior to treatment. As provided herein, treatment can include one dosing session or multiple dosing sessions, and reduction in ocular neovascularization area (e.g., CNV area), in one embodiment, is assessed after individual dosing sessions, or multiple dosing sessions. In a further embodiment, the ocular neovascularization area (e.g., CNV area) is reduced by at least about 5%, or at least about 10%, or at least about 15%, or at least about 20%, or at least about 25%, or at least about 30%, or at least about 35%, or at least about 40%, or at least about 45%, or at least about 50%, as measured by fluorescein angiography.

In one embodiment of a method for treating an ocular disorder in the eye of a patient in need thereof with an immunoconjugate dimer, for example, a method for treating wet AMD, diabetic retinopathy, diabetic macular edema, or choroidal neovascularization secondary to an ocular disorder such as wet AMD provided herein, the retinal thickness of the treated eye is reduced in the eye of the patient, as compared to the retinal thickness prior to treatment, as measured by optical coherence tomography (OCT) As provided herein, treatment can include one dosing session or multiple dosing sessions, and reduction in retinal thickness, in one embodiment, is assessed after individual dosing sessions, or multiple dosing sessions. In a further embodiment, the retinal thickness is reduced by at least about 5%, or at least about 10%, or at least about 15%, or at least about 20%, or at least about 25%, or at least about 30%, or at least about 35%, or at least about 40%, or at least about 45%, or at least about 50%, as measured by OCT. In a further embodiment, the decreased retinal thickness is decreased central retinal subfield thickness (CST), decreased center point thickness (CPT), or decreased central foveal thickness (CFT).

In one embodiment of a method of the present disclosure, the patient exhibits a decrease in the size, volume, and/or thickness of the ocular melanoma subsequent to the one or more dosing sessions, as compared to the size, volume, and/or thickness of the ocular melanoma prior to the one or more dosing sessions. In a further embodiment, the decrease in size, volume, and/or thickness of the ocular melanoma is measured by ultrasound, high-resolution ultrasound biomicroscopy, magnetic resonance imaging, and/or computed axial tomography.

In one embodiment of an ocular melanoma treatment method provided herein, the patient exhibits a decrease in swelling and/or fluid accumulation beneath the retina or choroid subsequent to the one or more dosing sessions, as compared to the swelling and/or fluid accumulation beneath the retina or choroid prior to the one or more dosing session. In a further embodiment, the decrease in swelling and/or fluid accumulation is measured by ocular coherence tomography.

In one embodiment of a method of the present disclosure, the patient exhibits a decrease in leakage or blockage of blood vessels in the eye subsequent to the one or more dosing sessions, as compared to the leakage or blockage of blood vessels in the eye prior to the one or more dosing sessions. In a further embodiment, the decrease is measured by fluorescein angiography or indocyanine green angiography.

In one embodiment of a method of the present disclosure, the patient exhibits a decrease in the size and/or number of iris spots subsequent to the one or more dosing sessions, as compared to the size and/or number of iris spots prior to the one or more dosing sessions. In a further embodiment, the decrease is measured by gonioscope, slit-lamp biomicroscope, and/or ophthalmoscope.

The immunoconjugates provided herein are amenable for use in any disease or disorder in which pathological neovascularization is implicated. For example, in one aspect, an immunoconjugate dimer provided herein is administered to the eye of a patient in need of treatment of ocular melanoma. In one embodiment, the treatment comprises multiple dosing sessions of the immunoconjugate dimer. As provided throughout, the immunoconjugate dimer comprises monomer subunits that each include a mutated human Factor Vila (FVIIa) protein conjugated to the human immunoglobulin Gi (IgG1) Fc domain. In one embodiment, a monomer unit of the immunoconjugate dimer has the amino acid sequence of SEQ ID NO: 4 or 5. In a specific embodiment, a monomer unit of the immunoconjugate dimer has the amino acid sequence of SEQ ID NO: 4. In another specific embodiment, a monomer unit of the immunoconjugate dimer has the amino acid sequence of SEQ ID NO: 5. In another specific embodiment, a monomer unit of the immunoconjugate dimer comprises the targeting domain of SEQ ID NO: 1, 2, 12, 33, or 34 and comprises an effector domain (inclusive of a hinge region) of SEQ ID NO: 13-18 and 26-32.

In one embodiment, the method of treating ocular melanoma comprises preventing, inhibiting or reversing tumor-associated neovascularization in the eye of the patient in need of treatment. In a further embodiment, neovascularization is reversed by at least about 10%, at least about 20%, at least about 30% or at least about 40% after treatment, as compared to the choroidal neovascularization that was present in the afflicted eye of the patient prior to treatment.

In one embodiment of a method for treating an ocular melanoma with an immunoconjugate dimer, the patient subjected to the treatment method substantially maintains his or her vision subsequent to the treatment (e.g., the single dosing session or multiple dosing sessions), as measured by losing fewer than 15 letters in a best-corrected visual acuity (BCVA) measurement, compared to the patient's BCVA measurement prior to undergoing treatment. In a further embodiment, the patient loses fewer than 10 letters, fewer than 8 letters, fewer than 6 letters or fewer than 5 letters in a BCVA measurement, compared to the patient's BCVA measurement prior to undergoing treatment.

In another embodiment of a method for treating an ocular melanoma with an immunoconjugate dimer, the patient subjected to the treatment method substantially maintains his or her vision subsequent to the treatment (e.g., the single dosing session or multiple dosing sessions), as measured by gaining 15 or more letters in a best-corrected visual acuity (BCVA) measurement, compared to the patient's BCVA prior to the multiple dosing sessions. In a further embodiment, the patient gains about 15 letters or more, about 20 letters or more, about 25 letters or more in a BCVA measurement, compared to the patient's BCVA measurement prior to undergoing treatment. In even a further embodiment, the patient gains from about 15 to about 30 letters, from about 15 letters to about 25 letters or from about 15 letters to about 20 letters in a BCVA measurement, compared to the patient's BCVA measurement prior to undergoing treatment.

In one embodiment of a method for treating an ocular melanoma in the eye of a patient in need thereof with an immunoconjugate dimer provided herein, the ocular neovascularization area of the eye of the patient is reduced in the eye of the patient, as compared to the ocular neovascularization area prior to treatment. As provided herein, treatment can include one dosing session or multiple dosing sessions, and reduction in ocular neovascularization area (e.g., CNV area), in one embodiment, is assessed after individual dosing sessions, or multiple dosing sessions. In a further embodiment, the ocular neovascularization area (e.g., CNV area) is reduced by at least about 5%, or at least about 10%, or at least about 15%, or at least about 20%, or at least about 25%, or at least about 30%, or at least about 35%, or at least about 40%, or at least about 45%, or at least about 50%, as measured by fluorescein angiography.

In one embodiment of a method for treating an ocular melanoma in the eye of a patient in need thereof with an immunoconjugate dimer provided herein, the retinal thickness of the treated eye is reduced in the eye of the patient, as compared to the retinal thickness prior to treatment, as measured by optical coherence tomography (OCT). As provided herein, treatment can include one dosing session or multiple dosing sessions, and reduction in retinal thickness, in one embodiment, is assessed after individual dosing sessions, or multiple dosing sessions. In a further embodiment, the retinal thickness is reduced by at least about 5%, or at least about 10%, or at least about 15%, or at least about 20%, or at least about 25%, or at least about 30%, or at least about 35%, or at least about 40%, or at least about 45%, or at least about 50%, as measured by OCT. In a further embodiment, the decreased retinal thickness is decreased central retinal subfield thickness (CST), decreased center point thickness (CPT), or decreased central foveal thickness (CFT).

In one embodiment of a method of the present disclosure, the patient exhibits a decrease in the size, volume, and/or thickness of the ocular melanoma subsequent to the one or more dosing sessions, as compared to the size, volume, and/or thickness of the ocular melanoma prior to the one or more dosing sessions. In a further embodiment, the decrease in size, volume, and/or thickness of the ocular melanoma is measured by ultrasound, high-resolution ultrasound biomicroscopy, magnetic resonance imaging, and/or computed axial tomography.

In one embodiment of an ocular melanoma treatment method provided herein, the patient exhibits a decrease in swelling and/or fluid accumulation beneath the retina or choroid subsequent to the one or more dosing sessions, as compared to the swelling and/or fluid accumulation beneath the retina or choroid prior to the one or more dosing session. In a further embodiment, the decrease in swelling and/or fluid accumulation is measured by ocular coherence tomography.

In one embodiment of a method of the present disclosure, the patient exhibits a decrease in leakage or blockage of blood vessels in the eye subsequent to the one or more dosing sessions, as compared to the leakage or blockage of blood vessels in the eye prior to the one or more dosing sessions. In a further embodiment, the decrease is measured by fluorescein angiography or indocyanine green angiography.

In one embodiment of a method of the present disclosure, the patient exhibits a decrease in the size and/or number of iris spots subsequent to the one or more dosing sessions, as compared to the size and/or number of iris spots prior to the one or more dosing sessions. In a further embodiment, the decrease is measured by gonioscope, slit-lamp biomicroscope, and/or ophthalmoscope.

In one embodiment, solid tumors are treated with immunoconjugates of the present disclosure with the systemic administration of an immunoconjugate. In some embodiments, systemic administration of an immunoconjugate is preferential over a localized administration in view of an inaccessible solid tumor. In some embodiments, an immunoconjugate is administered both systemically and locally.

In other embodiments, the immunoconjugates of the present invention are amenable for use in any disease or disorder where aberrant expression of TF is observed. For example, in one aspect, an immunoconjugate provided herein is administered to a patient in need of treatment of TF-expressing glioma.

In some embodiments, the immunoconjugates provided herein are amenable for use in any disease or disorder in which neovascularization is implicated. For example, in one aspect, an immunoconjugate provided herein is administered to the eye of a patient in need of treatment of wet age-related macular degeneration (AMD). In another aspect, an immunoconjugate provided herein is administered to the eye of a patient in need of treatment of ocular melanoma. In one embodiment, the treatment comprises multiple dosing sessions of the immunoconjugate.

In some embodiments, in cancer treatments, the immunoconjugate is used for treating a variety of cancers, particularly primary or metastatic solid tumors, including melanoma, renal, prostate, breast, ovarian, brain, neuroblastoma, head and neck, pancreatic, bladder, endometrial and lung cancer. In some embodiments, the immunoconjugate is further used for treating the following cancers: Ewing tumor, Wilms tumor, vulvar, vaginal, uterine sarcoma, thyroid, thymus, testicular, stomach, small intestine, Merkel cell, basal cell carcinoma, squamous cell carcinoma, Waldenstrom macroglobulinemia, soft tissue sarcoma, salivary gland, rhabdomyosarcoma, retinoblastoma, prostate, pituitary, penile, pancreatic, glioma, gynecological (serous, clear cell, endometriod, undifferentiated ovarian), osteosarcoma, oropharyngeal, non-Hodgkin lymphoma, neuroblastoma, nasopharyngeal, nasal, paranasal, multiple myeloma, lymphoma, lung (small cell, non-small cell, carcinoid tumor), liver, leukemia (acute lymphocytic, acute myeloid, chronic lymphocytic, chronic myelomonocytic), laryngeal, hypopharyngeal, kidney, Kaposi sarcoma, Hodgkin disease, gestational trophoblastic disease, gastrointestinal (stromal tumor, carcinoid tumor), gallbladder, eye, esophagus, endometrial, colorectal, cervical, breast, brain, CNS, bone, bladder, bile duct, anal, and adrenal.

In one embodiment, the cancer is a gynecological cancer. In a further embodiment, the gynecological cancer is serous, clear cell, endometriod or undifferentiated ovarian cancer.

The immunoconjugate, in one embodiment, is employed to target the tumor vasculature, particularly vascular endothelial cells, and/or tumor cells. Without wishing to be bound by theory, targeting the tumor vasculature offers several advantages for cancer immunotherapy with one or more of the immunoconjugates described herein, as follows: (i) some of the vascular targets including tissue factor should be the same for all tumors; (ii) immunoconjugates targeted to the vasculature do not have to infiltrate a tumor mass in order to reach their targets; (iii) targeting the tumor vasculature should generate an amplified therapeutic response, because each blood vessel nourishes numerous tumor cells whose viability is dependent on the functional integrity of the vessel; and (iv) the vasculature is unlikely to develop resistance to an immunoconjugate, because that would require modification of the entire endothelium layer lining a vessel. Unlike previously described antiangiogenic methods that inhibit new vascular growth, immunoconjugates provided herein elicit a cytolytic response to the neovasculature.

### Formulations, Administration and Dosing

Provided herein are administration and dosing embodiments for the immunoconjugates or compositions comprising the immunoconjugates. In some embodiments, administration may be local or systemic, depending upon the type of pathological condition involved in the therapy.

In one embodiment, the immunoconjugate is administered as a solution or a suspension. The immunoconjugate dimer, in one embodiment, comprises arginine or protein A. In a further embodiment, the immunoconjugate dimer comprises arginine. In an even further embodiment, the arginine is present in the composition from about 20 mM to about 40 mM, e.g., at 25 mM. Other components of the composition, in one embodiment, included HEPES, sodium chloride, polysorbate-80, calcium chloride, or a combination thereof. In one embodiment, histidine is present.

In one embodiment, the immunoconjugate dimer is administered in a dose of between 1 μg and 1500 μg, In one embodiment, the immunoconjugate dimer is administered in a dose of between 10 μg and 600 μg, 10 μg and 500 μg, 10 μg and 400 μg, 10 g and 300 μg, 10 μg and 200 μg, 10 μg and 100 μg, 10 μg and 50 μg, 50 μg and 600 μg, 50 μg and 500 μg, 50 μg and 400 μg, 50 μg and 300 μg, 50 μg and 200 μg, 50 μg and 100 g, 100 μg and 600 μg, 100 μg and 500 μg, 100 μg and 400 μg, 100 μg and 300 μg, 100 μg and 200 μg, 200 μg and 600 μg, 200 μg and 500 μg, 200 μg and 400 μg, 200 μg and 300 μg, 300 μg and 500 μg, 300 μg and 400 μg, or 400 μg and 500 μg. In one embodiment, the immunoconjugate dimer is administered at single dose of 300 μg. In one embodiment, the immunoconjugate dimer is administered with multiples doses of 300 μg each. In one embodiment, the immunoconjugate dimer is administered at single dose of 600 μg. In one embodiment, the immunoconjugate dimer is administered with multiples doses of 600 μg each.

In one embodiment, the immunoconjugate dimer is administered in a dose consisting of about 1 μg, 10 μg, about 20 μg, about 30 μg, about 40 μg, about 50 μg, about 60 μg, about 70 μg, about 80 μg, about 90 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, about 500 μg, about 525 μg, about 550 μg, about 575 μg, about 600 μg, about 625 μg, about 650 μg, about 675 μg, about 700 μg, about 725 μg, about 750 μg, about 775 μg, about 800 μg, about 825 μg, about 850 μg, 875 μg, about 900 μg, about 925 μg, about 950 μg, about 975 μg, about 1000 μg, about 1100 μg, about 1200 μg, about 1300 μg, about 1400 μg, or about 1500 μg.

In some embodiments, a single dose of the immunoconjugate dimer is administered. In some embodiments, two or more doses of the immunoconjugate dimer is administered. In an exemplary embodiment, two doses of 300 μg each are administered, spaced by an interval of 1 week (7 days). In an exemplary embodiment, two doses of 600 μg each are administered, spaced by an interval of 1 week (7 days).

In one embodiment, the immunoconjugate dimer is administered in a solute volume of between 10 μL and 200 μL, 10 μL and 180 μL, 10 μL and 160 μL, 10 μL and 140 μL, 10 μL and 120 μL, 10 μL and 100 μL, 10 μL and 80 μL, 10 μL and 60 μL, 10 μL and 40 μL, 10 μL and 20 μL, 10 μL and 15 μL, 20 μL and 200 μL, 20 μL and 180 μL, 20 μL and 160 μL, 20 μL and 140 μL, 20 μL and 120 μL, 20 μL and 100 μL, 20 μL and 80 μL, 20 μL and 60 μL, 20 μL and 40 μL, 40 μL and 200 μL, 40 μL and 180 μL, 40 μL and 160 μL, 40 μL and 140 μL, 40 μL and 120 μL, 40 μL and 100 μL, 40 μL and 80 μL, 40 μL and 60 μL, 60 μL and 200 μL, 60 μL and 180 μL, 60 μL and 160 μL, 60 μL and 140 μL, 60 μL and 120 μL, 60 μL and 100 μL, 60 μL and 80 μL, 80 μL and 200 μL, 80 μL and 180 μL, 80 μL and 160 μL, 80 μL and 140 μL, 80 μL and 120 μL, 80 μL and 100 μL, 100 μL and 200 μL, 100 μL and 180 μL, 100 μL and 160 μL, 100 μL and 140 μL, 100 μL and 120 μL, 120 μL and 200 μL, 120 μL and 180 μL, 120 μL and 160 μL, 120 μL and 140 μL, 140 μL and 200 μL, 140 μL and 180 μL, 140 μL and 160 μL, 160 μL and 200 μL, 160 μL and 180 μL, or 180 μL and 200 μL.

In one embodiment, the immunoconjugate dimer is administered in a solute volume consisting of about 10 μL, about 15 μL, about 20 μL, about 25 μL, about 30 μL, about 35 μL, about 40 μL, about 45 μL, about 50 μL, about 55 μL, about 60 μL, about 65 μL, about 70 μL, about 75 μL, about 80 μL, about 85 μL, about 90 μL, about 95 μL, or about 100 μL.

Exemplary compositions of the present invention are provided in Tables 3-5 below.

TABLE 3
Exemplary immunoconjugates

| Component | Concentration |
|---|---|
| Immunoconjugate | 3 mg/mL in 15 mM HEPES |
| NaCl | 150 mM |
| Arginine | 25 mM, pH 7.4 |
| Polysorbate-80 | 0.01% |
| CaCl₂ | 5 mM |

TABLE 4
Exemplary immunoconjugates

| Component | Concentration |
|---|---|
| Immunoconjugate of SEQ ID NO: 4 or 5 | 3 mg/mL in 15 mM HEPES |
| NaCl | 150 mM |
| Arginine | 25 mM, pH 7.4 |
| Polysorbate-80 | 0.01% |
| CaCl₂ | 5 mM |

TABLE 5
Exemplary immunoconjugate compositions

| Component | Component |
|---|---|
| Immunoconjugate | Immunoconjugate |
| HEPES | HEPES |
| NaCl | NaCl |
| Arginine | Glycine |
| Polysorbate-80 | Polysorbate-80 |
| CaCl₂ | CaCl₂ |

It is to be understood that in particular embodiments, the formulations may comprise arginine, or may comprise histidine; or may comprise arginine and histidine, at various concentrations. The formulations may additionally, or alternatively, comprise other amino acids, or amino acid derivatives.

Administration methods encompassed by the methods provided herein include, but are not limited to intravitreal injection, suprachoroidal injection, topical administration (e.g., eye drops), intravenous and intratumoral administration, or any other method depending on the condition or disease to be treated. In another embodiment, administration is via intravenous, intramuscular, intratumoral, subcutaneous, intrasynovial, intraocular, intraplaque, intrathecal, or intradermal injection of the immunoconjugate or of a replication-deficient adenoviral vector, or other viral vectors carrying a cDNA encoding a secreted form of the immunoconjugate. In one embodiment, a systemic administration may occur via parenteral injection. In one embodiment, the patient in need of treatment is administered one or more fusion proteins via intravitreal, intravenous or intratumoral injection, or injection at other sites, of one or more immunoconjugate proteins. Alternatively, in one embodiment, a patient in need of treatment is administered one or more fusion proteins via intravenous or intratumoral injection, or injection at other sites, of one or more expression vectors carrying a cDNA encoding a secreted form of one or more of the fusion proteins provided herein. In some embodiments, the patient is treated by intravenous or intratumoral injection of an effective amount of one or more replication-deficient adenoviral vectors, or one or more adeno-associated vectors carrying cDNA encoding a secreted form of one or more types of immunoconjugate proteins.

In some embodiments, a systemic administration of the one-armed FVII-Fc immunoconjugate may occur via intravenous, intramuscular, subcutaneous, or intradermal injection. In some embodiments, systemic administration, as utilized herein, is the administration of a substance of the present disclosure to a patient in need, wherein the substance enters the circulatory system and is dispersed throughout the patient in need. In one embodiment, a systemic administration of a substance of the present disclosure includes the distribution of the substance across the blood-brain barrier.

In one embodiment, a method of intravitreal injection is employed. In a further embodiment, aseptic technique is employed when preparing the one-armed FVII-Fc immunoconjugate for injection, for example, via the use of sterile gloves, a sterile drape and a sterile eyelid speculum (or equivalent). In one embodiment, the patient is subjected to anesthesia and a broad-spectrum microbicide prior to the injection.

In one embodiment, intravitreal injection of one or more of the immunoconjugates provided herein is prepared by withdrawing the vial contents of the immunoconjugate composition solution through a 5-micron, 19-gauge filter needle attached to a 1-cc tuberculin syringe. The filter needle in a further embodiment, is then discarded and replaced with a sterile 30-gauge×½-inch needle for the intravitreal injection. The contents of the vial are expelled until the plunger tip is aligned with the line on the syringe that marks the appropriate dose for delivery.

In another embodiment, the treatment methods provided herein comprise multiple dosing sessions. In a further embodiment, the multiple dosing sessions are multiple intraocular injections of one of the immunoconjugates described herein. The multiple dosing sessions, in one embodiment comprise two or more, three or more, four or more or five or more dosing sessions. In a further embodiment, each dosing session comprises intraocular injection of one of the immunoconjugates described herein, or intratumoral injection of one of the immunoconjugates described herein (i.e., either as the expressed protein or via a vector encoding the soluble fusion protein). In another embodiment, each of said two or more, three or more, four or more or five or more dosing sessions may comprise a systemic dosing.

In one embodiment, from about 2 to about 24 dosing sessions are employed, for example, from about 2 to about 24 intraocular dosing sessions (e.g., intravitreal or suprachoroidal injection). In a further embodiment, from about 3 to about 30, or from about 5 to about 30, or from about 7 to about 30, or from about 9 to about 30, or from about 10 to about 30, or from about 12 to about 30 or from about 12 to about 24 dosing sessions are employed.

In one embodiment, where multiple dosing sessions are employed, the dosing sessions are spaced apart by from about 10 days to about 60 days, or from about 10 days to about 50 days, or from about 10 days to about 40 days, or from about 10 days to about 30 days, or from about 10 days to about 20 days. In another embodiment, where multiple dosing sessions are employed, the dosing sessions are spaced apart by from about 20 days to about 60 days, or from about 20 days to about 50 days, or from about 20 days to about 40 days, or from about 20 days to about 30 days. In even another embodiment, the multiple dosing sessions are bi-weekly (e.g., about every 14 days), monthly (e.g., about every 30 days), or bi-monthly (e.g., about every 60 days). In yet another embodiment, the dosing sessions are spaced apart by about 28 days.

### Co-Administration

In some embodiments, the immunoconjugates described herein are administered in a co-therapeutic regimen to treat a patient for one of the aforementioned diseases or disorders, for example, to treat wet AMD or another ocular disease associated with neovascularization. The method involves (either concurrent or non-concurrent) administration of a second active agent. In one embodiment, the second active agent is administered in the same composition as the immunoconjugate. However, in another embodiment, second active agent is administered in a separate composition. In one embodiment, the second active agent is a neovascularization inhibitor, an angiogenesis inhibitor, or a cancer chemotherapeutic. In one embodiment, the second active agent is a checkpoint inhibitor (anti-CTLA4, anti-PD1/PDL1). In another embodiment, the second active agent is an immunotherapeutic/immunotherapy.

In one embodiment, the second active agent is a vascular endothelial growth factor (VEGF) inhibitor, a VEGF receptor inhibitor, a platelet derived growth factor (PDGF) inhibitor, or a PDGF receptor inhibitor.

In another embodiment, the second active agent which is a neovascularization inhibitor is an integrin antagonist, a selectin antagonist, an adhesion molecule antagonist (e.g., antagonist of intercellular adhesion molecule (ICAM)-1, ICAM-2, ICAM-3, platelet endothelial adhesion molecule (PCAM), vascular cell adhesion molecule (VCAM)), lymphocyte function-associated antigen 1 (LFA-1)), a basic fibroblast growth factor antagonist, a vascular endothelial growth factor (VEGF) modulator, or a platelet derived growth factor (PDGF) modulator (e.g., a PDGF antagonist). In one embodiment of determining whether a patient is likely to respond to an integrin antagonist, the integrin antagonist is a small molecule integrin antagonist, for example, an antagonist described by Paolillo et al. (Mini Rev Med Chem, 2009, volume 12, pp. 1439-1446, incorporated by reference in its entirety), or a leukocyte adhesion-inducing cytokine or growth factor antagonist (e.g., tumor necrosis factor-α (TNF-α), interleukin-1β (IL-13), monocyte chemotactic protein-1 (MCP-1) and a vascular endothelial growth factor (VEGF)), as described in U.S. Pat. No. 6,524,581, incorporated by reference in its entirety herein.

In another embodiment, the second active agent which is a neovascularization inhibitor is one or more of the following angiogenesis inhibitors: interferon gamma 1β, interferon gamma 113 (Actimmune®) with pirfenidone, ACUHTR028, αVβ5, aminobenzoate potassium, amyloid P, ANG1122, ANG1170, ANG3062, ANG3281, ANG3298, ANG4011, anti-CTGF RNAi, Aplidin, astragalus membranaceus extract with salvia and schisandra chinensis, atherosclerotic plaque blocker, Azol, AZXI00, BB3, connective tissue growth factor antibody, CT140, danazol, Esbriet, EXC001, EXC002, EXC003, EXC004, EXC005, F647, FG3019, Fibrocorin, Follistatin, FTO 11, a galectin-3 inhibitor, GKT137831, GMCTOI, GMCT02, GRMD01, GRMD02, GRN510, Heberon Alfa R, interferon α-2β, ITMN520, JKB119, JKBI21, JKB122, KRX168, LPA1 receptor antagonist, MGN4220, MIA2, microRNA 29a oligonucleotide, MMI0100, noscapine, PBI4050, PBI4419, PDGFR inhibitor, PF-06473871, PGN0052, Pirespa, Pirfenex, pirfenidone, plitidepsin, PRM151, Pxl02, PYN17, PYN22 with PYN17, Relivergen, rhPTX2 fusion protein, RXI109, secretin, STX100, TGF-3 Inhibitor, transforming growth factor, β-receptor 2 oligonucleotide, VA999260, XV615, or a combination thereof.

In another embodiment, the second active agent is an endogenous angiogenesis inhibitor. In a further embodiment, the endogenous angiogenesis inhibitor is endostatin, a 20 kDa C-terminal fragment derived from type XVIII collagen, angiostatin (a 38 kDa fragment of plasmin), or a member of the thrombospondin (TSP) family of proteins. In a further embodiment, the angiogenesis inhibitor is a TSP-1, TSP-2, TSP-3, TSP-4 and TSP-5. Methods for determining the likelihood of response to one or more of the following angiogenesis inhibitors are also provided a soluble VEGF receptor, e.g., soluble VEGFR-1 and neuropilin 1 (NPR1), angiopoietin-1, angiopoietin-2, vasostatin, calreticulin, platelet factor-4, a tissue inhibitor of metalloproteinase (TIMP) (e.g., TIMP1, TIMP2, TIMP3, TIMP4), cartilage-derived angiogenesis inhibitor (e.g., peptide troponin I and chrondomodulin I), a disintegrin and metalloproteinase with thrombospondin motif 1, an interferon (IFN) (e.g., IFN-α, IFN-0, IFN-γ), a chemokine, e.g., a chemokine having the C—X—C motif (e.g., CXCL10, also known as interferon gamma-induced protein 10 or small inducible cytokine B10), an interleukin cytokine (e.g., IL-4, IL-12, IL-18), prothrombin, antithrombin III fragment, prolactin, the protein encoded by the TNFSF15 gene, osteopontin, maspin, canstatin, proliferin-related protein.

In one embodiment, one or more of the following neovascularization inhibitors is administered with the immunoconjugate described herein: angiopoietin-1, angiopoietin-2, angiostatin, endostatin, vasostatin, thrombospondin, calreticulin, platelet factor-4, TIMP, CDAI, interferon α, interferon β, vascular endothelial growth factor inhibitor (VEGI) meth-1, meth-2, prolactin, VEGI, SPARC, osteopontin, maspin, canstatin, proliferin-related protein (PRP), restin, TSP-1, TSP-2, interferon gamma 10, ACUHTR028, αVβ5, aminobenzoate potassium, amyloid P, ANG1122, ANG1170, ANG3062, ANG3281, ANG3298, ANG4011, anti-CTGF RNAi, Aplidin, astragalus membranaceus extract with salvia and schisandra chinensis, atherosclerotic plaque blocker, Azol, AZX100, BB3, connective tissue growth factor antibody, CT140, danazol, Esbriet, EXC001, EXC002, EXC003, EXC004, EXC005, F647, FG3019, Fibrocorin, Follistatin, FT011, a galectin-3 inhibitor, GKT137831, GMCT01, GMCT02, GRMD01, GRMD02, GRN510, Heberon Alfa R, interferon α-2β, ITMN520, JKB119, JKB121, JKB122, KRX168, LPA1 receptor antagonist, MGN4220, MIA2, microRNA 29a oligonucleotide, MM1000, noscapine, PB14050, PBI4419, PDGFR inhibitor, PF-06473871, PGN0052, Pirespa, Pirfenex, pirfenidone, plitidepsin, PRMI51, Px102, PYN17, PYN22 with PYN17, Relivergen, rhPTX2 fusion protein, RXI109, secretin, STX100, TGF-β Inhibitor, transforming growth factor, 3-receptor 2 oligonucleotide, VA999260, XV615, or a combination thereof.

Yet another co-therapy embodiment includes administration of one of the immunoconjugates described herein with one or more of the following: pazopanib (Votrient), sunitinib (Sutent), sorafenib (Nexavar), axitinib (Inlyta), ponatinib (Iclusig), vandetanib (Caprelsa), cabozantinib (Cometrig), bevacizumab (Avastin), ramucirumab (Cyramza), regorafenib (Stivarga), ziv-aflibercept (Zaltrap), or a combination thereof. In yet another embodiment, the angiogenesis inhibitor is a VEGF inhibitor. In a further embodiment, the VEGF inhibitor is axitinib, cabozantinib, aflibercept, brivanib, tivozanib, ramucirumab or motesanib. In one exemplary embodiment, the co-therapy comprises administration of the one-armed immunoconjugate dimer (ICON-1.5) and aflibercept.

In other embodiments, additional co-therapies includes administration of one of the immunoconjugates described herein with one or more of the following immune checkpoint inhibitors: ipilimiumab, nivoluman, pembrolizumab, and other molecules affecting the tumor microenvironment.

In one embodiment, the angiogenesis inhibitor is ranibizumab or bevacizumab. In a further embodiment, the angiogenesis in inhibitor is ranibizumab. In an exemplary embodiment, the co-therapy comprises administration of the one-armed immunoconjugate dimer (ICON-1.5) and ranibizumab. In even a further embodiment, ranibizumab is administered at a dosage of 0.5 mg or 0.3 mg per dosing session, and is administered as indicated in the prescribing information for LUCENTIS.

In one embodiment, the co-therapy comprises administration of an antagonist of a member of the platelet derived growth factor (PDGF) family, for example, a drug that inhibits, reduces or modulates the signaling and/or activity of PDGF-receptors (PDGFR). For example, the PDGF antagonist, in one embodiment, is an anti-PDGF aptamer, an anti-PDGF antibody or fragment thereof, an anti-PDGFR antibody or fragment thereof, or a small molecule antagonist. In one embodiment, the PDGF antagonist is an antagonist of the PDGFR-α or PDGFR-β. In one embodiment, the PDGF antagonist is the anti-PDGF-β aptamer E10030, sunitinib, axitinib, sorefenib, imatinib, imatinib mesylate, nintedanib, pazopanib HCl, ponatinib, MK-2461, dovitinib, pazopanib, crenolanib, PP-121, telatinib, imatinib, KRN 633, CP 673451, TSU-68, Ki8751, amuvatinib, tivozanib, masitinib, motesanib diphosphate, dovitinib dilactic acid, linifanib (ABT-869).

### Pharmaceutical Compositions

The present application provides pharmaceutical compositions comprising any one of the immunoconjugate dimers described herein with one or more pharmaceutically acceptable excipients. In some embodiments, the composition is sterile. The pharmaceutical compositions generally comprise an effective amount of the immunoconjugate dimer.

### Kits and Articles of Manufacture

The present application provides kits comprising an immunoconjugate dimer described herein. In some embodiments, the kits further contain a pharmaceutically acceptable excipient and instruction manual. In one specific embodiment, the kit comprises any one or more of the therapeutic compositions described herein, with one or more pharmaceutically acceptable excipients. The present application also provides articles of manufacture comprising any one of the therapeutic compositions or kits described herein. Examples of an article of manufacture include vials (including sealed vials).

The invention will be more fully understood by reference to the following examples. They should not, however, be construed as limiting the scope of the invention. It is understood that the examples and embodiments described herein are for illustrative purposes only.

### EXAMPLES

The present invention is further illustrated by reference to the following Examples. The Examples, like the embodiments described above, are illustrative and are not to be construed as restricting the scope of the invention in any way.

### Example 1—Expression and Purification of ICON-1.5 in Patients with CNV Secondary to Age-Related Macular Degeneration

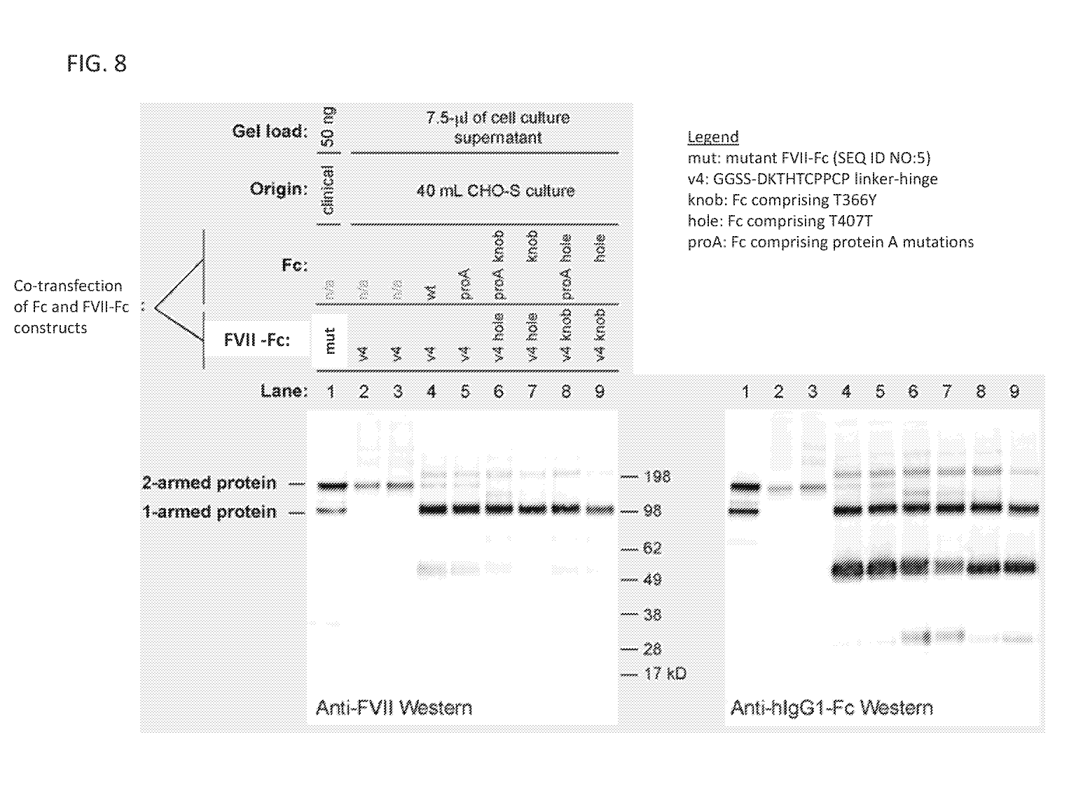

In this study, the ICON-1.5 immunoconjugate was expressed and purified. Mammalian CHO-S cells were utilized for production of a variety of constructs related to the present invention. As noted in FIG. 8, constructs were co-transformed into CHO-S cells, and the cell culture supernatant was collected and evaluated for the presence of proteins relating to the present invention, specifically those that were bound Anti-FVII antibodies and Anti-human IgG1 Fc antibodies.

One day post-transfection, the cells were fed with the appropriate reagents. Once viability was between 70% and 80%, the cell supernatant was collected by centrifugation and depth filtration.

The following supernatants were run on Western blots in duplicate, with one of the runs exposed to anti-FVII and the other exposed to Anti-human IgG1 Fc. Lane 1 comprises supernatant from just transfection of a construct comprising mutant FVII-Fc (SEQ ID NO:4), while lanes 2 and 3 each have supernatant from cells having been transfected with just a single construct comprising variant 4 (v4) of the FVII-Fc, which comprises the mutant FVII (SEQ ID NO:12) fused to a GGSS linker (SEQ ID NO:11) connected to the lower hinge of IgG1 followed by the remaining IgG1 sequence (SEQ ID NO:13). Lane 4 comprises supernatant from cells co-transfected with the v4 construct and a second construct, the Fc-only expression construct (SEQ ID NO:13). Lane 5 comprises supernatant from cells co-transfected with the v4 construct and a second construct expressing Fc with a protein A mutation (SEQ ID NO:14). Lane 6 comprises supernatant from cells co-transfected with the v4 hole construct (v4, but featuring the Fc hole mutation, wherein SEQ ID NO:13 is replaced with SEQ ID NO:17), and a second construct expressing Fc with both the protein A and knob mutations (SEQ ID NO:16). Lane 7 comprises supernatant from cells co-transfected with the v4 hole construct, and a second construct expressing the Fc with a knob mutation (SEQ ID NO:15). Lane 8 comprises supernatant from cells co-transfected with the v4 knob construct (v4, but featuring the Fc knob mutation, wherein SEQ ID NO:13 was replaced with SEQ ID NO:15), and a second construct expressing the Fc with protein A and hole mutations (SEQ ID NO:18). Lane 9 comprises supernatant from cells co-transfected with the v4 knob construct, and a second construct expressing the Fc protein with a hole mutation (SEQ ID NO:17).

The Western blots shoed that the one-armed ICON-1.5 immunoconjugate was expressed in a greater amount than the two-armed immunoconjugate, particularly in the cells that utilized the knob-hole mutant Fc heterodimers.

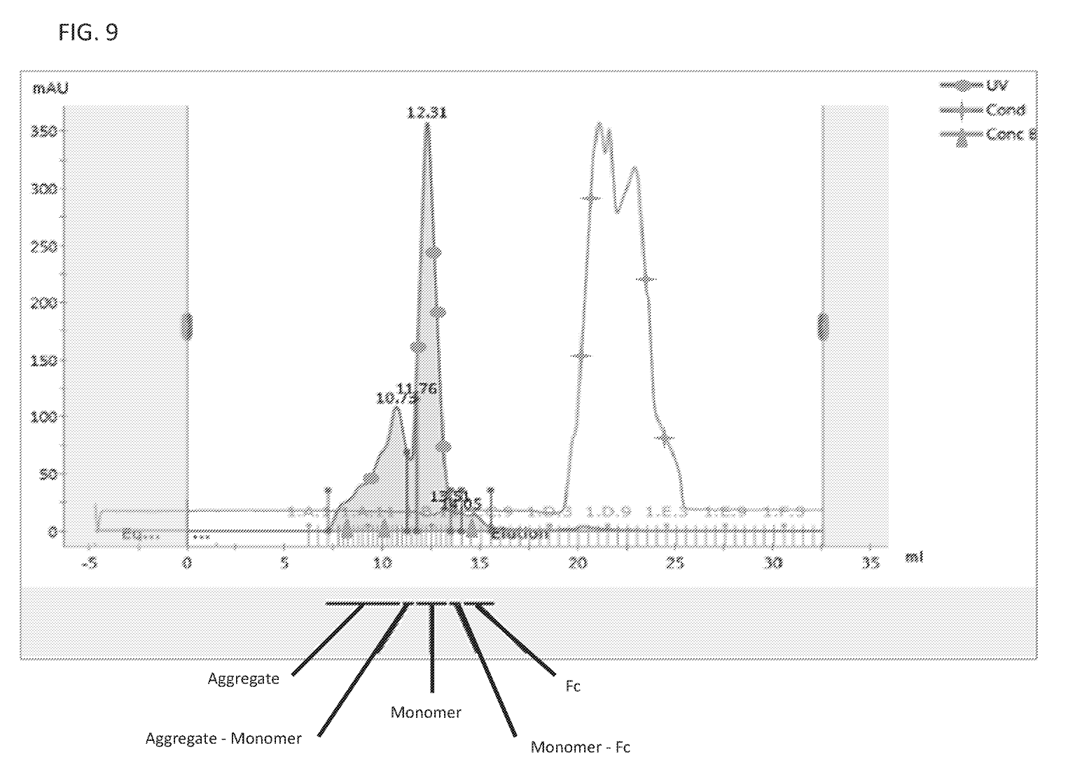

The immunoconjugates were isolated from the supernatant through size exclusion chromatography, as evidenced by FIG. 9, which depicts the dominant peak as comprising the ICON-1.5 immunoconjugate, labeled as “Monomer” in FIG. 9. Thus, the ICON-1.5 immunoconjugate can be isolated and further purified from the cellular supernatant.

### Example 2—Characterization of the ICON-1.5 One-Armed FVII-Fc Immunoconjugate

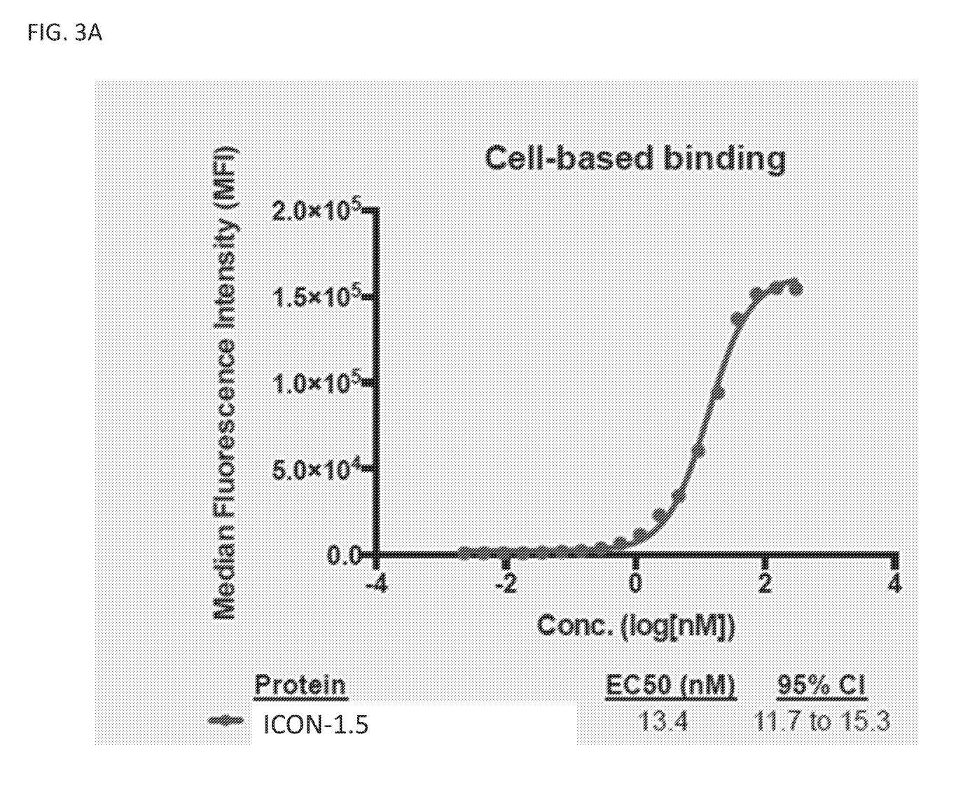

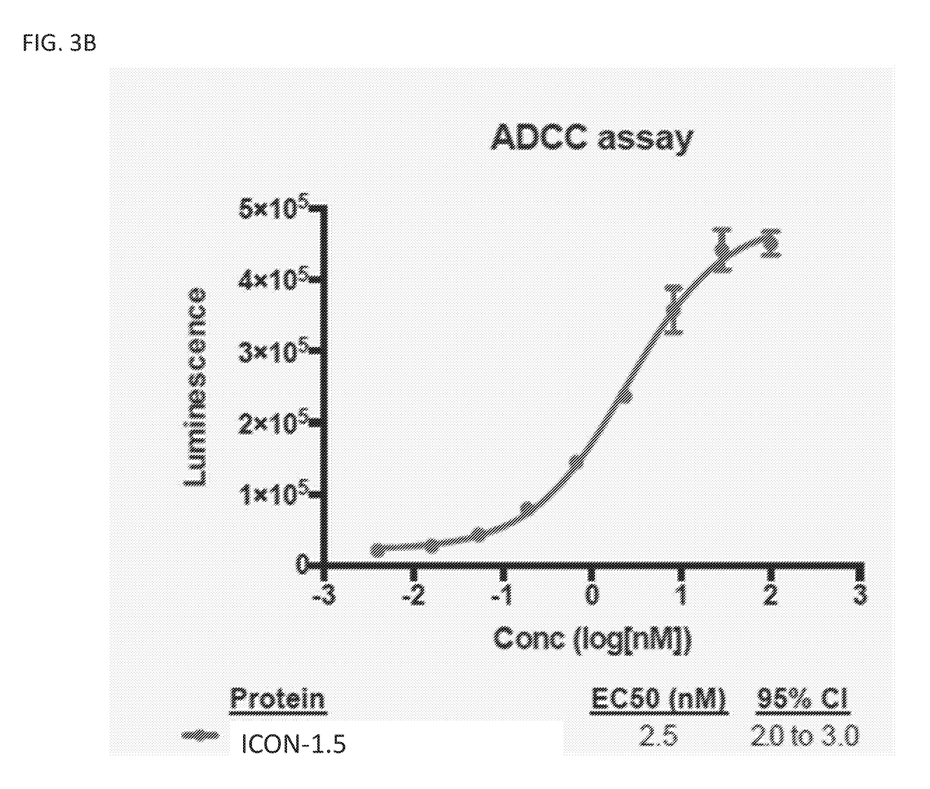

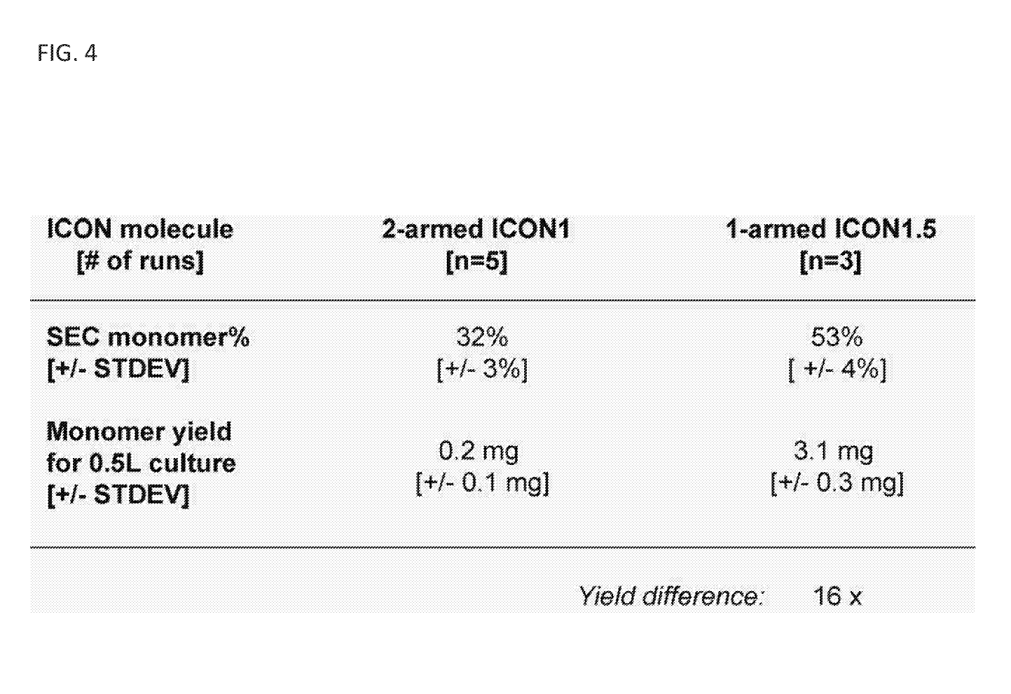

The ICON-1.5 one-armed FVII-Fc immunoconjugate was characterized for cell-based binding affinity of the antibody. Cells from human epidermoid carcinoma cell line A431 (ATCC CRL-1555™, Manassas, Va.) expressing TF were utilized. Antibody binding was performed using serially diluted protein (18 different concentrations). Binding of the test proteins was detected using an appropriate Phycoerythrin-labelled anti-Fc secondary antibody. Flow cytometry was utilized to determine antibody-cell binding affinity. All measurements were carried out of viable cells as determined using a TO-PRO-3 Iodide staining assay to identify and eliminate dead cells from the binding affinity assay. Binding was reported as the mean fluorescence intensity (MFI) of viable cells (See FIG. 3A).

ICON-1.5 one-armed FVII-Fc immunoconjugate variant was further characterized with regard to the ability to activate, in immunological effector cells, antibody-dependent cell-mediated cytotoxicity (ADCC). The Promega ADCC reporter bioassay kit (cat#G7018, Fitchburg, Wis.) was utilized to determine ADCC activity of the antibody, following the protocol provided by the manufacturer. The data readout from this assay is luminescence signal from an inducible NFAT response element that drives the expression of firefly luciferase. Following engagement with the Fc region of a relevant antibody bound to a target cell, ADCC bioassay effector cells expressing a specific FcγR transduce intracellular signals resulting in NFAT-mediate luciferase activity (See FIG. 3B). The cell line utilized in the ADCC assays is of a TF expressing cell line, which demonstrates that even in the absence of vasculature, given that these are in vitro assays, there is clear induction of ADCC with the one-armed FVII-Fc immunoconjugate.

### Example 3—Production Yield of the ICON-1.5 One-Armed FVII-Fc Immunoconjugate Versus the ICON-1 Two-Armed FVII-Fc Immunoconjugate

The ICON-1.5 immunoconjugate and the ICON-1 immunoconjugate was transiently expressed in CHO-S mammalian cells in separate experiments. The production yield of the ICON-1.5 immunoconjugate was 16-fold higher than the production yield of the ICON-1 immunoconjugate.

### Example 4—Characterization of the ICON-1.5 Binding and Activity of the One-Armed FVII-Fc Immunoconjugate Versus the Two-Armed FVII-Fc Immunoconjugate in CHO-S Derived Immunoconjugates

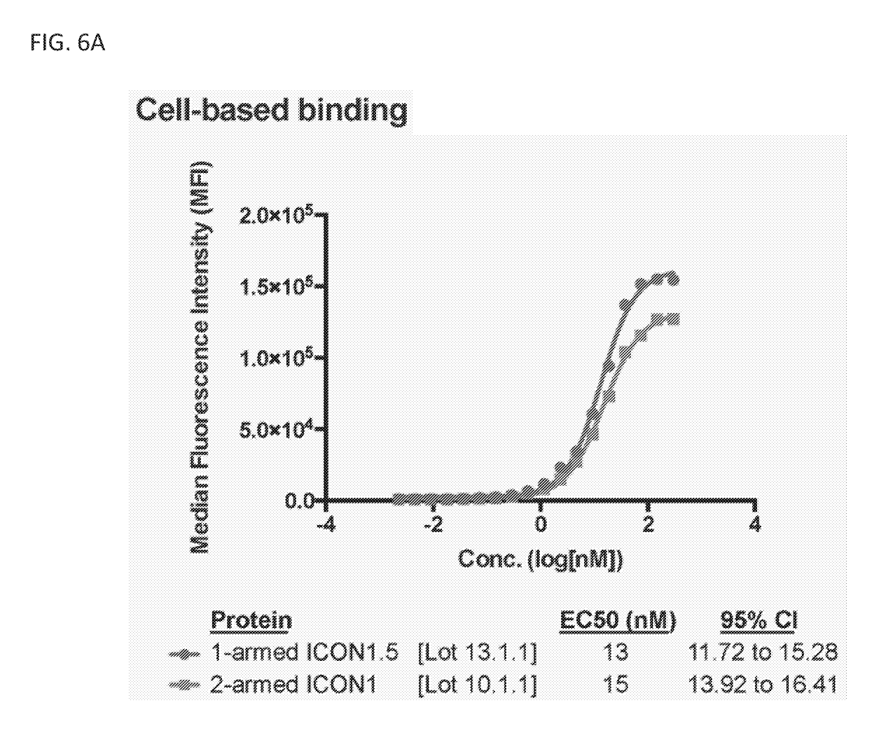

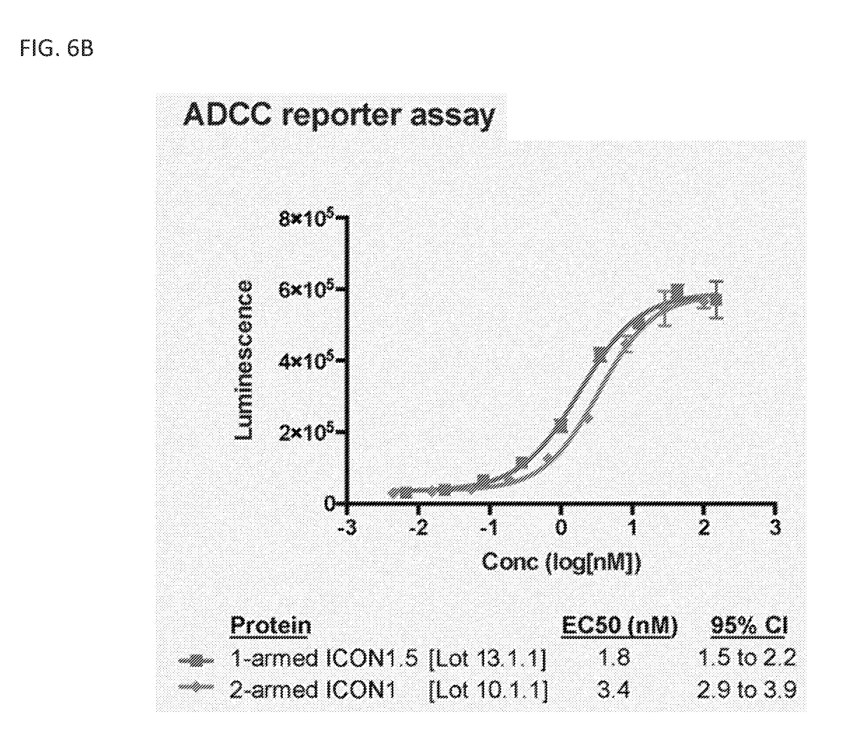

The ICON-1.5 one-armed immunoconjugate was characterized alongside the ICON-1 two-armed immunoconjugate with regard to a cell-based binding affinity of the antibody. Cells from human epidermoid carcinoma cell line A431 (ATCC CRL-1555™, Manassas, Va.) expressing TF were utilized. Antibody binding was performed using serially diluted protein (18 different concentrations). Binding of the test proteins was detected using an appropriate AF488-labelled anti-Fc secondary antibody. Flow cytometry was utilized to determine antibody-cell binding affinity. All measurements were carried out of viable cells as determined using a propidium iodide (PI) staining assay to identify and eliminate dead cells from the binding affinity assay. Binding was reported as the mean fluorescence intensity (MFI) of viable cells (See FIG. 6A). The results demonstrate that ICON-1.5 binds at least as effectively as ICON-1.

The ICON-1.5 one-armed immunoconjugate was further characterized alongside the ICON-1 two-armed immunoconjugate with regard to the ability to activate, in immunological effector cells, antibody-dependent cell-mediated cytotoxicity (ADCC). The Promega ADCC reporter bioassay kit (cat#G7018, Fitchburg, Wis.) was utilized to determine ADCC activity of the antibody, following the protocol provided by the manufacturer. The data readout from this assay is luminescence signal from an inducible NFAT response element that drives the expression of firefly luciferase. Following engagement with the Fc region of a relevant antibody bound to a target cell, ADCC bioassay effector cells expressing a specific FcγR transduce intracellular signals resulting in NFAT-mediated luciferase activity (See FIG. 6B). The results demonstrate that ICON-1.5 exhibits an equivalent activity in the ability to activate ADCC as ICON-1. The cell line utilized in the ADCC assays is of a TF expressing cell line, which demonstrates that even in the absence of vasculature, given that these are in vitro assays, there is clear induction of ADCC with the one-armed FVII-Fc immunoconjugate.

### Example 5—Characterization of the ICON-1.5 Binding and Activity of the One-Armed FVII-Fc Immunoconjugate Versus the Two-Armed FVII-Fc Immunoconjugate in HEK293 Derived Immunoconjugates

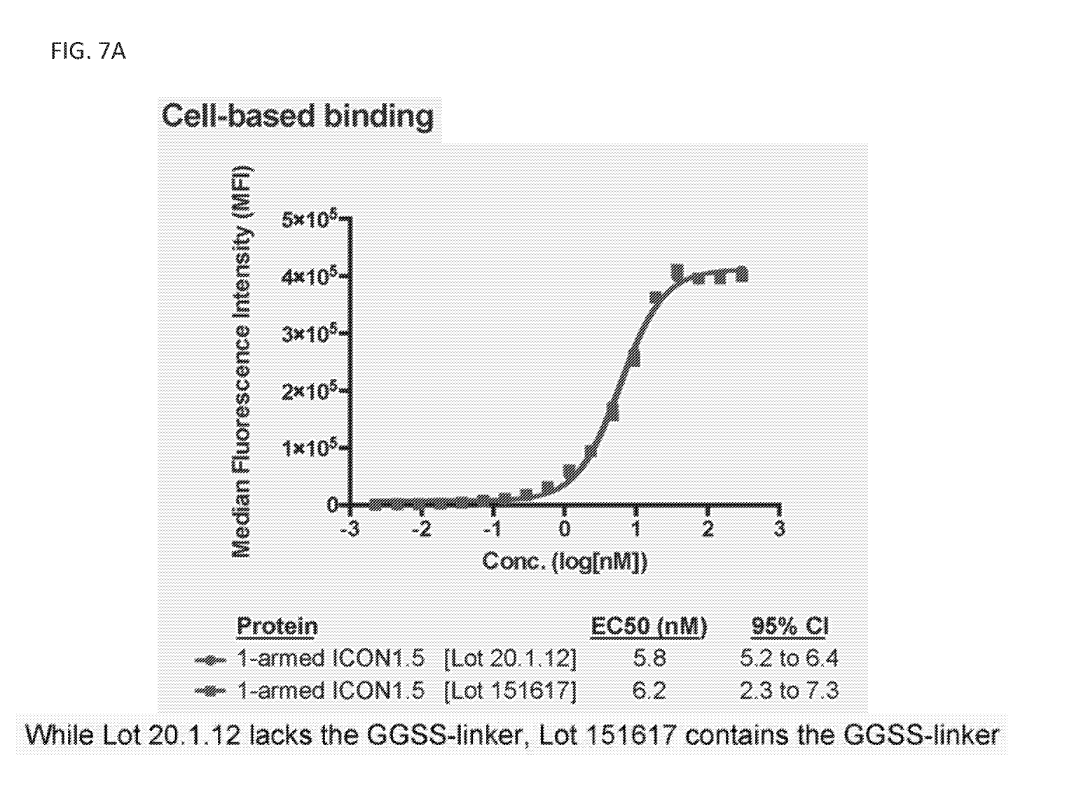

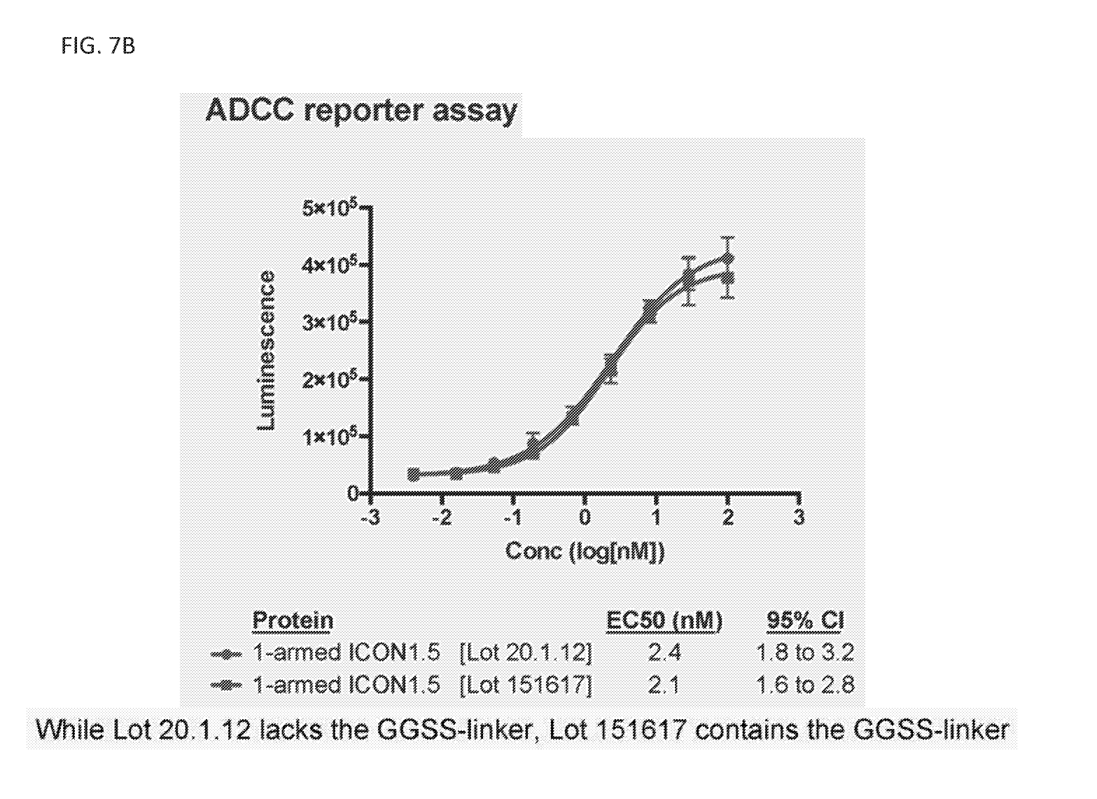

The HEK293 derived ICON-1.5 one-armed immunoconjugate comprising a GGSS (SEQ ID NO:11) linker sequence was characterized alongside the HEK293 derived ICON-1.5 one-armed immunoconjugate lacking a GGSS (SEQ ID NO:11) linker sequence with regard to a cell-based binding affinity of the antibody. Cells from human epidermoid carcinoma cell line A431 (ATCC CRL-1555™, Manassas, Va.) expressing TF were utilized. Antibody binding was performed using serially diluted protein (18 different concentrations). Binding of the test proteins was detected using an appropriate Phycoerythrin-labelled anti-Fc secondary antibody. Flow cytometry was utilized to determine antibody-cell binding affinity. All measurements were carried out of viable cells as determined using a TO-PRO-3 Iodide staining assay to identify and eliminate dead cells from the binding affinity assay. Binding was reported as the mean fluorescence intensity (MFI) of viable cells (See FIG. 7A). The results demonstrate that the ICON-1.5 immunoconjugate lacking the GGSS (SEQ ID NO:11) linker sequence was not discernable from the ICON-1.5 comprising said linker sequence with regard to binding.

The HEK293 derived ICON-1.5 one-armed immunoconjugate comprising a GGSS (SEQ ID NO:11) linker sequence was characterized alongside the HEK293 derived ICON-1.5 one-armed immunoconjugate lacking a GGSS (SEQ ID NO:11) linker sequence with regard to the ability to activate, in immunological effector cells, antibody-dependent cell-mediated cytotoxicity (ADCC). The Promega ADCC reporter bioassay kit (cat#G7018, Fitchburg, Wis.) was utilized to determine ADCC activity of the antibody, following the protocol provided by the manufacturer. The data readout from this assay is luminescence signal from an inducible NFAT response element that drives the expression of firefly luciferase. Following engagement with the Fc region of a relevant antibody bound to a target cell, ADCC bioassay effector cells expressing a specific FcγR transduce intracellular signals resulting in NFAT-mediated luciferase activity (See FIG. 7B). The results demonstrate that the ICON-1.5 immunoconjugate lacking the GGSS (SEQ ID NO:11) linker sequence was not discernable from the ICON-1.5 comprising said linker sequence with regard to activity in the ability to activate ADCC. The cell line utilized in the ADCC assays is of a TF expressing cell line, which demonstrates that even in the absence of vasculature, given that these are in vitro assays, there is clear induction of ADCC with the one-armed FVII-Fc immunoconjugate.

### Example 6—Randomized, Double-Masked, Multicenter, Active-Controlled Study Evaluating ICON-1.5 in Patients with CNV Secondary to Age-Related Macular Degeneration

In this study, the safety of intravitreal injections of one-armed FVII-Fc immunoconjugates, administered as monotherapy or in combination with ranibizumab (LUCENTIS) compared to ranibizumab monotherapy in patients with choroidal neovascularization (CNV) secondary to age-related macular degeneration (AMD) is assessed.

Additionally, the biological activity and pharmacodynamics effect of the one-armed FVII-Fc immunoconjugate, administered as monotherapy or in combination with ranibizumab (LUCENTIS) compared to ranibizumab monotherapy is assessed.

The study presented in this example is planned as a randomized, double-masked, active-controlled study. Patients are naïve to treatment for CNV. Patients are randomly assigned to one of the following three treatment arms in the selected study eye in a 1:1:1 ratio:
one-armed FVII-Fc immunoconjugate monotherapy (0.3 mg)+sham injection
ranibizumab monotherapy (0.5 mg)+sham injection
one-armed FVII-Fc immunoconjugate (0.3 mg)+ranibizumab (0.5 mg) combination therapy

Randomization is stratified by best-corrected visual acuity (BCVA) letter score in the study eye at baseline (≤54 letters versus ≥55 letters) and by study site.

Patients receive up to two intravitreal injections at each injection visit. In order to maintain the study mask among the treatment arms, a sham injection is employed in patients receiving monotherapy.

Patients are administered intravitreal injections in the study eye once every four weeks at months 0, 1 and 2. As of Month 3 (at Months 3, 4 and 5) patients are retreated according to their assigned treatment arm, based on their individual observed treatment response. The masked investigator uses the following retreatment criteria (based on the category of individual patient response) to determine if treatment is required at these visits:
Loss of ≥5 letters of BCVA due to AMD compared to the previous scheduled visit.
Independent of BCVA change, any anatomical evidenced of increased CNV activity (e.g., new or increased fluid and/or leakage, hemorrhage) compared to the previous scheduled visit.
No BCVA change compared to Baseline (Visit 2), but there is anatomical evidence of persistent CNV activity (e.g., same persistent fluid and CST compared to Baseline.

Rescue treatment with 0.5 mg of ranibizumab is administered to the study eye as an add-on therapy at any time during the 6-month treatment and follow-up period if either of the following conditions occur:
Loss of ≥15 letters of BCVA due to AMD compared to Baseline (Visit 2).
Loss of ≥10 letters from baseline (Visit 2) of BCVA due to AMD that is confirmed at two consecutive visits. Patients with a loss of ≥10 letters compared to baseline are requested to return within 7 days or as soon as possible for additional follow up at an unscheduled visit.

The masked physician will make the determination if rescue treatment is needed according to the above criteria.

If rescue treatment is administered to the study eye during a scheduled injection visit, to ensure that the study masking is maintained, the unmasked physician administers rescue treatment and the patient's scheduled study treatment/re-treatment is as follows.
one-armed FVII-Fc immunoconjugate monotherapy arm: immunoconjugate monomer (0.3 mg)+rescue therapy (0.5 mg ranibizumab).
ranibizumab monotherapy arm: ranibizumab (0.5 mg)+sham injection.
combination therapy: one-armed FVII-Fc immunoconjugate (0.3 mg)+ranibizumab (0.5 mg).

If rescue treatment is administered to the study eye at an unscheduled visit, the unmasked physician administers rescue treatment as requested.

If rescue treatment is administered to the study eye, the patient continues with the study visit schedule for the next visit in accordance with the protocol and continues receiving study treatment according to the assigned randomization arm.

Safety is evaluated by tracking of adverse events, clinical laboratory tests (serum chemistry, hematology and coagulation), vital signs measurements, abbreviated physical examinations, slit-lamp biomicroscopy, intraocular pressure (IOP) and dilated ophthalmoscopy. Pharmacodynamic and biological activity is measured by means of BCVA by ETDRS visual acuity chart, spectral-domain optical coherence tomography (sdOCT), color fundus photography (CFP), fundus fluorescein angiography (FA), fundus autofluorescence (FAF), contrast sensititivy, and microperimetry. Pharmacokinetic (PK) and immunogenicity is evaluated by means of measuring plasma concentrations of hI-con1 and anti-drug antibodies.

### Example 7—Binding Assay and ADCC Reporter Assay for CHO and 292-Derived ICON-1.5 Immunoconjugate Molecules

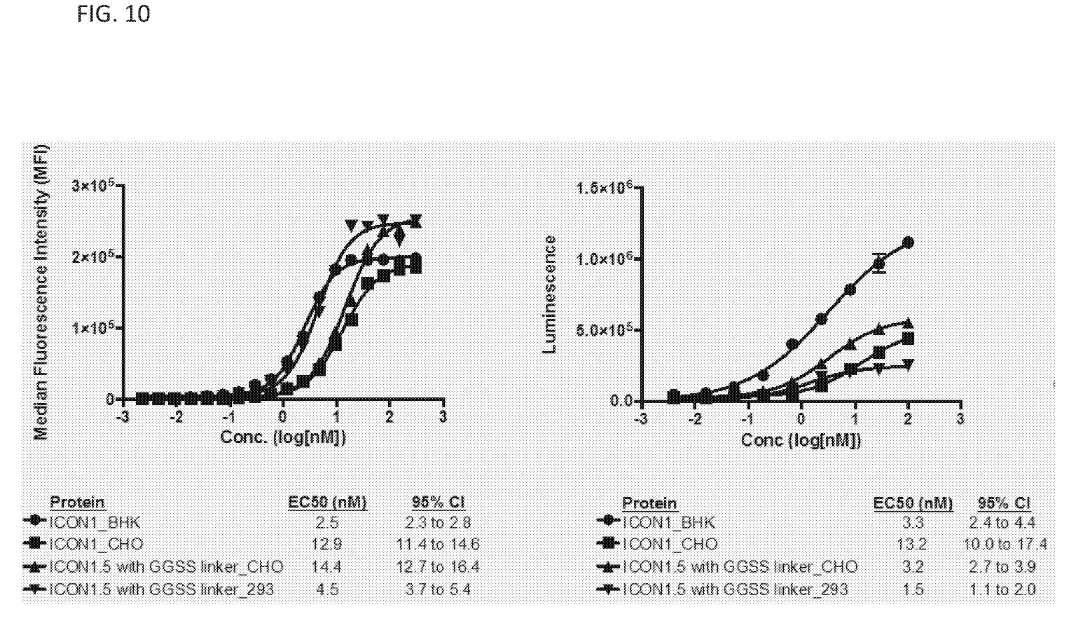

The one-armed ICON-1.5 immunoconjugate containing the GGSS linker sequence [SEQ ID no. 18 co-expressed with concatenate of SEQ ID nos. 12, 11 and 15] produced in CHO and 293 cells was characterized alongside the two-armed ICON-1 immunoconjugate produced in BHK and 293 cells to assess the effect of the production host cell line with regard to cell-based binding affinity of the Fc fusion protein. Cells from human epidermoid carcinoma cell line A431 (ATCC CRL-1555™, Manassas, Va.) expressing TF were utilized. Antibody binding was performed using serially diluted protein (18 different concentrations). Binding of the test proteins was detected by flow cytometry using an appropriate Phycoerythrin-labelled anti-Fc secondary antibody. All measurements were carried out on viable cells by using a TO-PRO-3 iodide staining assay to identify and eliminate dead cells from the binding affinity assay. Binding was reported as the mean fluorescence intensity (MFI) of viable cells and an EC50 value with a 95% confidence interval (95% CI) was derived using a four-parameter fit. See left panel of FIG. 10. The results demonstrate that ICON-1.5 produced in 293 binds TF similarly to ICON-1 produced in BHK, while both forms generated in CHO have reduced binding.

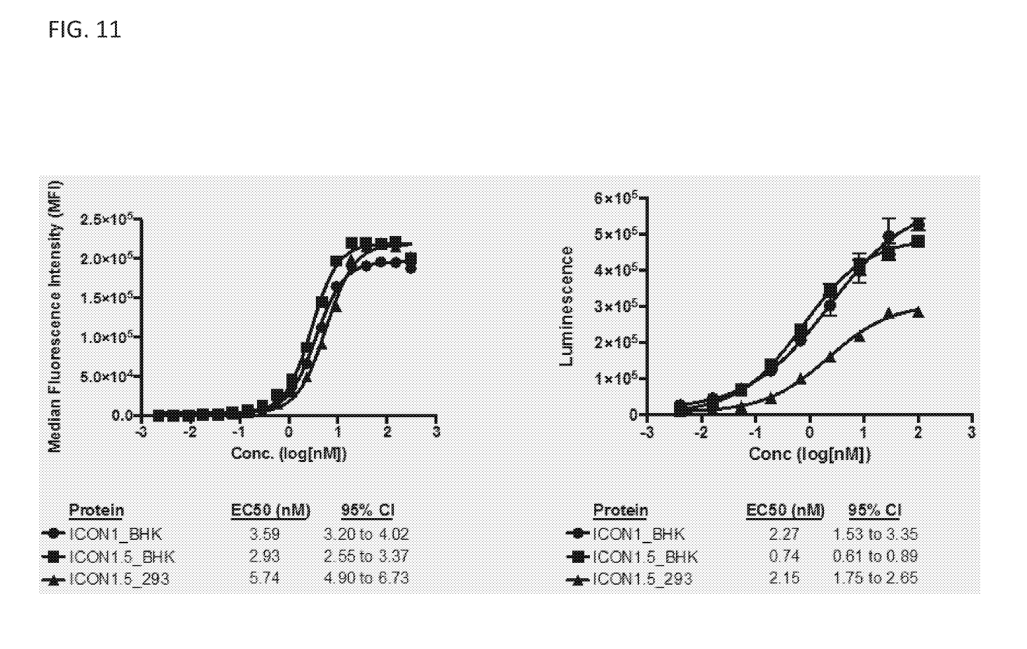

The one-armed ICON-1.5 immunoconjugate produced in CHO and 293 was further characterized alongside the two-armed ICON-1 immunoconjugate produced in BHK and in CHO with regard to the ability to activate, in immunological effector cells, antibody-dependent cell-mediated cytotoxicity (ADCC). The Promega ADCC reporter bioassay kit (cat#G7018, Fitchburg, Wis.) was utilized to determine ADCC activity of the Fc fusion protein, following the protocol provided by the manufacturer. The data readout from this assay is luminescence signal from an inducible NFAT response element that drives the expression of firefly luciferase. Following engagement with the Fc region of a relevant antibody or Fc fusion protein bound to a target cell, ADCC bioassay effector cells expressing a specific FcγR transduce intracellular signals resulting in NFAT-mediated luciferase activity (See right panel of FIG. 10. The results demonstrate that ICON-1 produced in BHK exhibits a more potent ADCC activity as observed by the lower EC50 and higher absolute signal than ICON-1 produced in CHO and the ICON-1.5 immunoconjugates produced in CHO and 293. Based on these findings, ICON-1.5 was also produced in BHK and underwent testing in the same types of assays. See FIG. 11.

### Example 8—Binding Assay and ADCC Reporter Assay for BHK and 292-Derived ICON-1.5 Immunoconjugate Molecules

The BHK derived one-armed ICON-1.5 immunoconjugate without a GGSS (SEQ ID NO:11) linker sequence (SEQ ID no. 18 co-expressed with concatenate of SEQ ID nos. 12 and 15) was characterized alongside the BHK derived ICON-1. Cells from human epidermoid carcinoma cell line A431 (ATCC CRL-1555™, Manassas, Va.) expressing TF were utilized. Antibody binding was performed using serially diluted protein (18 different concentrations). Binding of the test proteins was detected using an appropriate Phycoerythrin-labelled anti-Fc secondary antibody. Flow cytometry was utilized to determine antibody-cell binding affinity. All measurements were carried out on viable cells by using a TO-PRO-3 Iodide staining assay to identify and eliminate dead cells from the binding affinity assay. Binding was reported as the mean fluorescence intensity (MFI) of viable cells and an EC50 value with a 95% confidence interval (95% CI) was derived using a four-parameter fit. See left panel of FIG. 11. The results demonstrate that the BHK ICON-1.5 immunoconjugate was not discernible from the BHK ICON-1 with regard to binding.

The one-armed BHK derived ICON-1.5 immunoconjugate was characterized alongside the BHK derived ICON-1, with regard to the ability to activate, in immunological effector cells, antibody-dependent cell-mediated cytotoxicity (ADCC). The Promega ADCC reporter bioassay kit (cat#G7018, Fitchburg, Wis.) was utilized to determine ADCC activity of the Fc fusion protein, following the protocol provided by the manufacturer. The data readout from this assay is luminescence signal from an inducible NFAT response element that drives the expression of firefly luciferase. Following engagement with the Fc region of a relevant antibody or Fc fusion protein bound to a target cell, ADCC bioassay effector cells expressing a specific FcγR transduce intracellular signals resulting in NFAT-mediated luciferase activity. See right panel of FIG. 11. The results demonstrate that the BHK-derived ICON-1.5 immunoconjugate was not discernable from the BHK-derived ICON-1 with regard to activity in the ability to activate ADCC.

### Example 9—Cell-Based FXa Conversion Assay

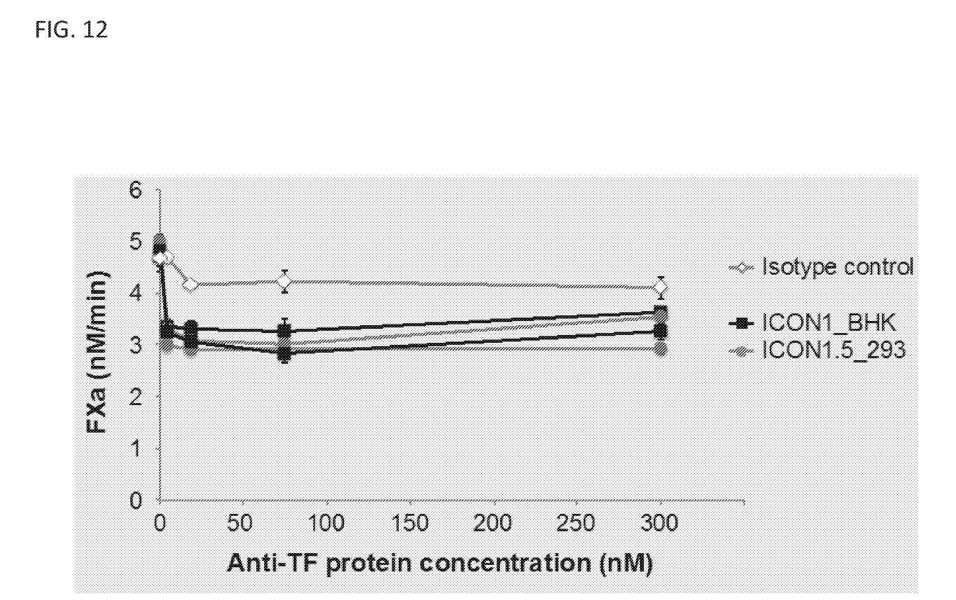

Binding of FVIIa to TF assembles FX zymogen leading to its conversion to active coagulation protease FXa which upon release from the TF:FVIIa complex promotes thrombin generation and blood clot formation. In order to determine the potential anticoagulant activity and ability to interfere with FVIIa-induced FX activation of ICON-1.5, cells were incubated with the indicated concentrations of ICON-1 and ICON 1.5 in the presence of 200 nM FX zymogen and 20 nM of FVIIa. After a 5 minute incubation, the reaction was quenched with ethylenediaminetetraacetic acid (EDTA) and the amount of FXa generated from FX was assessed by measuring the conversion of a FXa fluorogenic substrate called SN-7 (Haemtech SN-7 6-amino-1-naphthalenesulfonamide-based (ANSN) fluorogenic substrate). The results of this assay indicate that ICON-1 and ICON-1.5 have a comparable and limited ability to interfere with FVIIa-induced FX activation. See FIG. 12.

### Example 10—Recombinant TF Factor Xase Assay

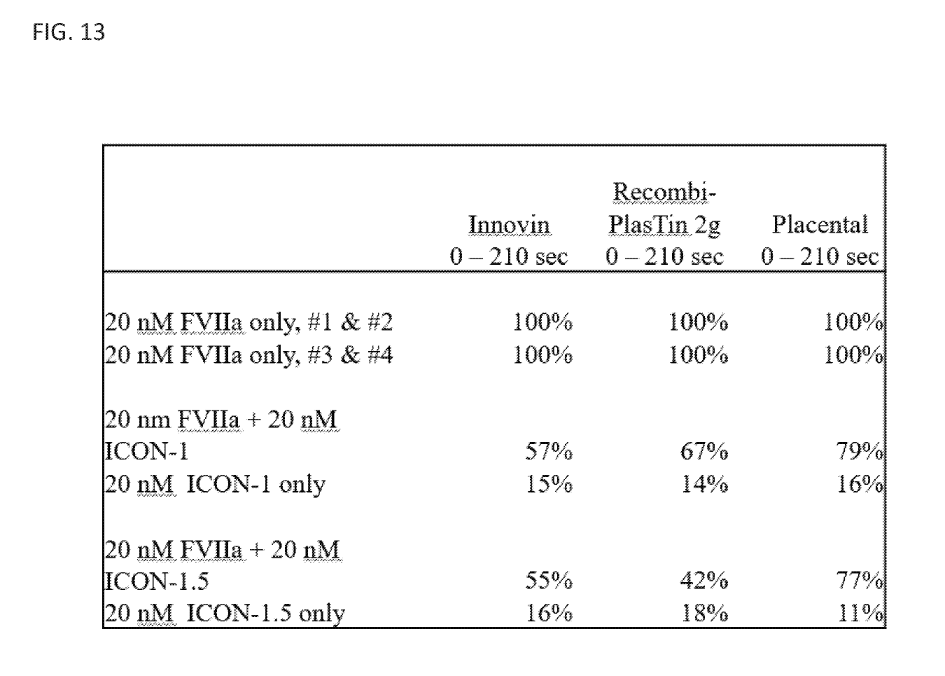

Xase is a proteolytic complex that consists of TF, FVIIa, phospholipids and Ca++. It cleaves Factor X to factor Xa. The basis of the assays is to incubate the Xase complex alone or in combination with test molecules and then add Factor X. If the test molecules are FVIIa anologues, such as ICON-1 and ICON-1.5, the reaction can be run in the absence of added FVIIa, to evaluate their procoagulant activity. The reaction is stopped at defined timepoints by addition of a solution that contains EDTA, which chelates Ca++. Spectrazyme FXa is then added to the chelated reaction mixture which contains FX along with FXa. The amount of Spectrazyme FXa cleaved is directly proportional to the amount of FXase present. If a test item interferes with the binding of FVIIa to TF, this will result in a decrease in FXase activity. The BHK derived one-armed ICON-1.5 immunoconjugate was characterized alongside the BHK derived ICON-1, with regard to the ability to modulate FXase using 3 different forms of TF: Innovin, RecombiplasTin 2G and placental TF. The data is expressed as percent of activity observed at 210 seconds with FVIIa added at 20 nM to the reaction mixture. The results indicate that ICON-1 and ICON-1.5 have comparable and weak ability to mediate FXase (less than 20% of activity observed with FVIIa). The results also indicate that ICON-1 and ICON-1.5 have similar inhibitory activity on the ability of recombinant FVIIa to interact with the three forms of TF in the in vitro FXase assay. See FIG. 13.

### Example 11—Secondary Antibody-Drug Conjugate Cytotoxicity Assay (ADC)

Secondary ADC assays allow to evaluate the ability of an antibody or a Fc fusion protein, following binding to its receptor, to internalize and mediate cell-killing without direct conjugation of a payload to this antibody or Fc fusion protein. A secondary antibody, in this case an anti-Fc Fab fragment coupled to the anti-tubulin agent MMAF (monomethyl auristatin F) or the DNA intercalator PNU-159268 (a derivative of nemorubicin), binds to the Fc portion of the molecule. If the cell internalizes the antibody or Fc Fusion protein in complex with the conjugated secondary antibody, dose-dependent cell killing is observed. The CellTiter-Glo 2.0 (CTG) assay, a method to determine the number of viable cells in culture based on quantitation of ATP (an indicator of metabolically active cells), was used.

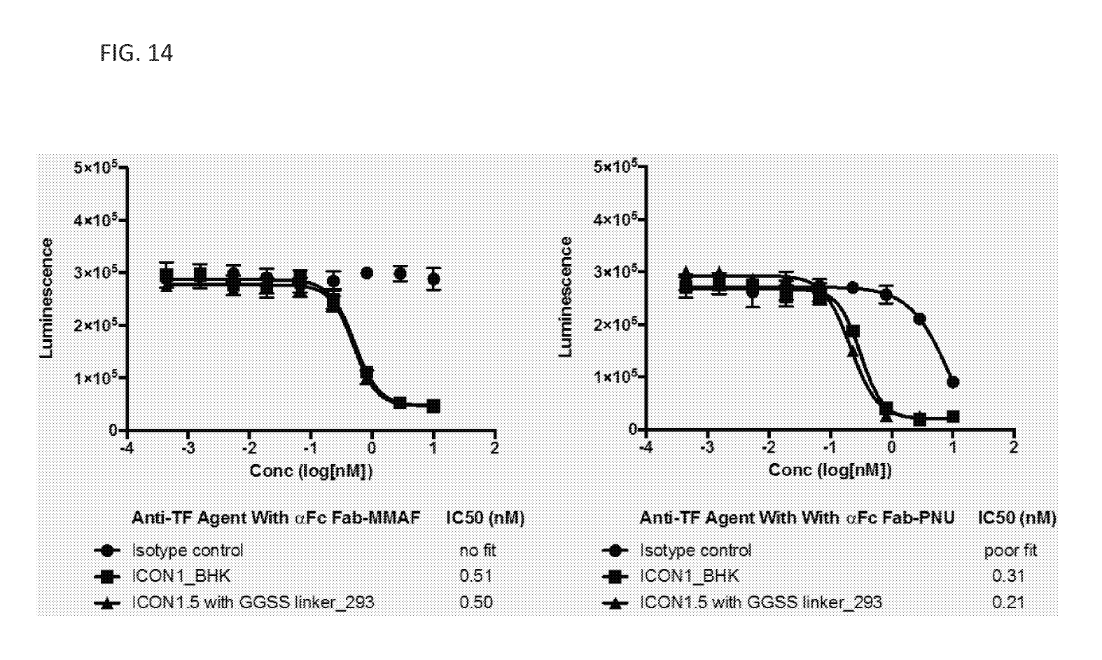

The ICON-1.5 immunoconjugate produced in 293 and ICON-1 produced in BHK were evaluated in secondary ADC assays with the Uveal Melanoma cell line Mel 290 which expresses high levels of TF. A 10-point, 3.5-fold titration starting at 10 nM of ICON-1 or ICON-1.5 and 60 nM of the secondary reagent was added to the cells. The cells were incubated for 3 days followed by evaluation of cell viability by the CTG assay. Comparable IC50's were observed for the one-armed ICON-1.5 and the two-armed ICON-1 immunoconjugates with both payloads, indicating a similar internalization rate. See FIG. 14.

### Example 12—Secondary Antibody-Drug Conjugate Cytotoxicity (ADC) Assay with Secondary Antibody from Multiple Cellular Backgrounds

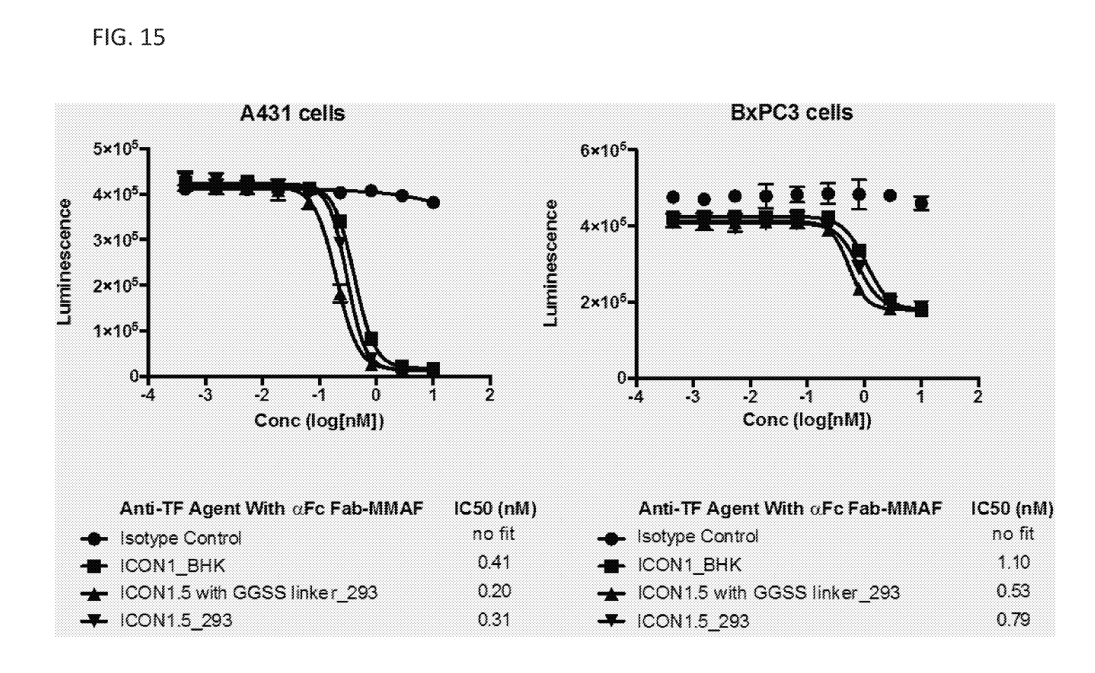

The one-armed ICON-1.5 immunoconjugate produced in 293 and the two-armed ICON-1 immunoconjugate produced in BHK were evaluated in ADC assays in which the secondary antibody is conjugated with the tubulin inhibitor MMAF in the epidermoid carcinoma cell line A431 and in the pancreatic adenocarcinoma cell line BxPC3; both cell lines express high levels of TF. A 10-point, 3.5-fold titration starting at 10 nM of ICON-1 and ICON-1.5 and 60 nM of the secondary reagent was added to the cells. The cells were incubated for 3 days followed by evaluation of cell viability by the CTG assay. Comparable IC50's were observed for the one-armed ICON-1.5 and the two-armed ICON-1 immunoconjugates with both payloads, indicating a similar internalization rate. See FIG. 15.

### Example 13—Secondary Antibody-Drug Conjugate Cytotoxicity (ADC) Assay with Secondary Antibody from MDA-MB-231 Triple Negative Breast Carcinoma Cell Line

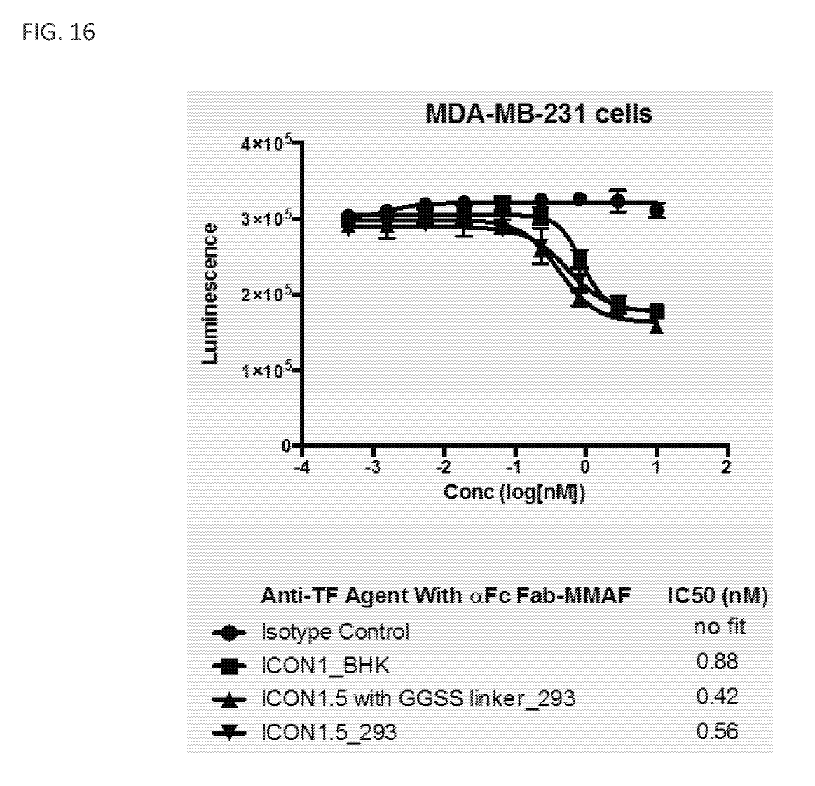

The one-armed ICON-1.5 immunoconjugate produced in 293 and the two-armed ICON-1 immunoconjugate produced in BHK were evaluated in ADC assays in which the secondary antibody is conjugated with the tubulin inhibitor MMAF in the triple negative breast carcinoma cell line MDA-MB-231 which expresses high levels of TF. A 10-point, 3.5-fold titration starting at 10 nM of ICON-1 or ICON-1.5 and 60 nM of the secondary reagent was added to the cells. The cells were incubated for 3 days followed by evaluation of cell viability by the CTG assay. Comparable IC50's were observed for the one-armed ICON-1.5 and the two-armed ICON-1 immunoconjugates with both payloads, indicating a similar internalization rate. See FIG. 16.

### Example 14—Effect of ICON-1 from BHK and ICON-1.5 from 293 on FVIIa-Induced Cell Signaling

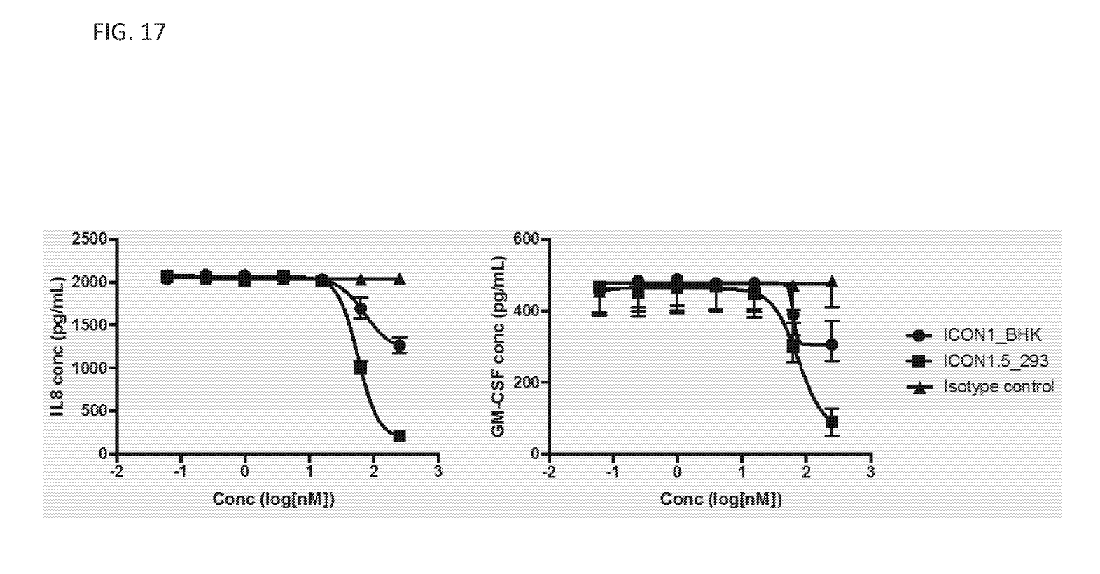

Binding of FVIIa to TF mediates activation of multiple signaling cascades, including proteolytic activation of PAR2 and MAPK signaling. These events lead to up-regulation of proinflammatory cytokines such as IL-8, GM-CSF and CXCL1, and result in promotion of neovascularization, tumor growth and metastasis. The effects of the one-armed ICON-1.5 immunoconjugate produced in 293 and the two-armed ICON-1 immunoconjugate produced in BHK were examined in FVIIa-induced cell-signaling. For these experiments, MDA-MB-231 cells were serum-starved for 2 hours, followed by a 30 min incubation with ICON-1 or ICON-1.5 (7-point, 4-fold titration, starting at 250 nM). FVIIa was then added to the Fc fusion protein containing media and cells for 5 hours and supernatants were collected. IL-8 and GM-CSF levels were measured by ELISA. The results indicate that the one-armed ICON-1.5 immunoconjugate has a more substantial inhibitory effect on cytokine signaling than the two-armed ICON-1 immunoconjugate. See FIG. 17.

### Example 15—Effect of Agents Directed Against TF on In Vivo Tumor Growth in a Murine Xenograft Study

To assess the potential effect of agents directed against TF on tumor growth in vivo, a xenograft study was performed in female athymic nude mice (Crl:NU(NCr)-Foxn1nu, Charles River). A431 cells were harvested during exponential growth and resuspended at a concentration of 1×10⁸ cells/mL in PBS. On the day of implantation, each test mouse received 1×10⁷ A431 cells (0.1 mL cell suspension) implanted subcutaneously in the right flank and tumor growth was monitored as the average size approached the target range of 100-125 mm3. Seven days later, designated as Day 1 of the study, the animals were sorted into 3 cohorts (n=10) with individual tumor volumes ranging from 88 to 172 mm3 and group mean tumor volumes from 108 to 140 mm3. To examine the effect on tumor growth of the one-armed ICON-1.5 immunoconjugate produced in 293 and the two-armed ICON-1 immunoconjugate produced in BHK, the proteins were delivered by intraperitoneal injection at a dose of 10 mg/kg, once a week for 3 weeks.

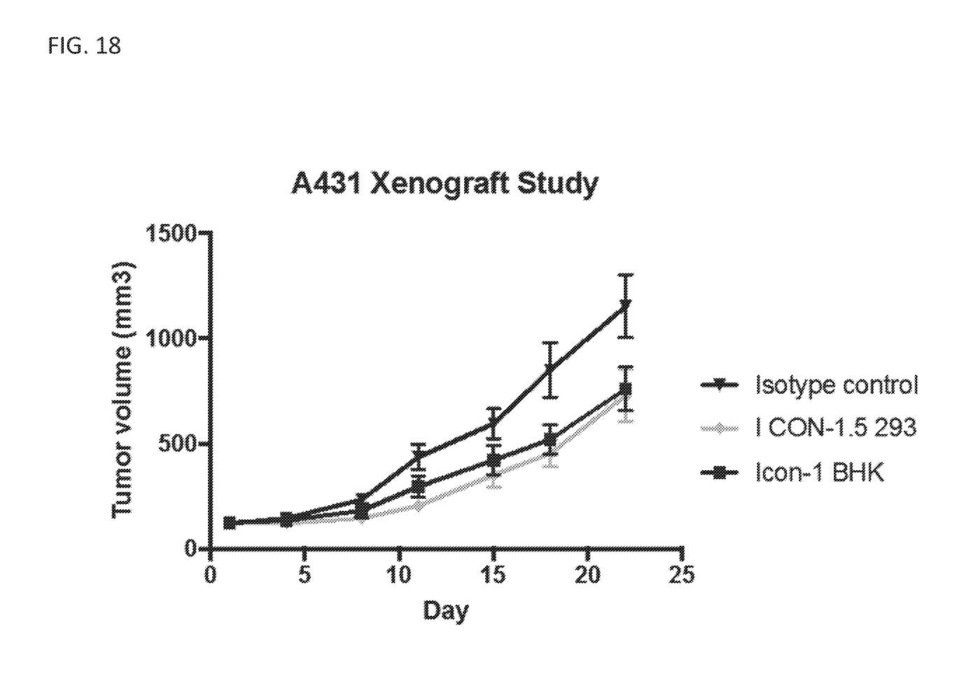

The tumors were measured twice weekly starting on Day 1 with a caliper in two dimensions to monitor size. The study endpoint was defined as a mean tumor volume of 2000 mm3 in the control group or 22 days, whichever came first. The study was terminated on Day 22. The results indicate that the one-armed immunoconjugate ICON-1.5 and the two-armed ICON-1 had a similar effect on tumor growth. See FIG. 18.

### Example 17—Pharmacological Study Evaluating the Efficacy of ICON-1.5 in a Rabbit Model of Laser-Induced Choroidal Neovascularization (CNV)

This study evaluates the efficacy of intravitreal injections of one-armed FVII-Fc immunoconjugates, administered as monotherapy or in combination with anti-VEGF agents such as ranibizumab (LUCENTIS) or aflibercept (EYLEA) compared to anti-VEGF monotherapy in a rabbit model of laser induced choroidal neovascularization (CNV). Four animals per group, and six groups. The groups consist of (1) vehicle, (2) 300 μg, (3) 600 μg, (4) 900 μg, (5) aflibercept 2.0 mg, and (6) aflibercept 2.0 mg+ICON-1 (ICON-1.5 in parallel study) at 600 μg.

Rabbits are lasered in both eyes (OU) on Day 0 (DO). Test articles and vehicle are dosed bilaterally via intravitreal (IVT) injection on D7. Ranibizumab (LUCENTIS) or aflibercept (EYLEA) are dosed on day 0 immediately after laser (DO). In the combination group (group 7), anti-VEGF agents are injected on DO and one-armed FVII-Fc immunoconjugates on D7.

Ocular Examination: Mydriasis for ocular examination is done using topical 1% tropicamide HCL (one drop in each eye 15 minutes prior to examination). Complete ocular examination (modified Hackett and McDonald) using a slit lamp biomicroscope and indirect ophthalmoscope to evaluate ocular surface morphology, anterior segment and posterior segment inflammation, cataract formation, and retinal changes are conducted by a veterinary ophthalmologist at baseline and D14.

Fluorescein angiography (FA): FA is done in both eyes of all animals on D7, D10, and D14 after laser. Mydriasis for FA is done using topical 1% Tropicamide HCL (one drop in each eye 15 minutes prior to examination). Full FA is performed for 1-5 minutes after intravenous sodium fluorescein injection (12 mg kg⁻¹). A reader analyzes the masked images obtained. The area of maximal fluorescein leakage is measured using Image J for each lesion.

All eyes are collected for in situ hybridization (ISH) and flat-mount analysis of choroidal vascularity detected by fluorescein isothiocyanate-dextran staining.

### Example 18—Pharmacological Study Evaluating the Efficacy of ICON-1.5 in a Swine Model of Laser-Induced Choroidal Neovascularization (CNV)

This study evaluates the efficacy of intravitreal injections of one-armed FVII-Fc immunoconjugates, administered as monotherapy or in combination with anti-VEGF agents such as ranibizumab (LUCENTIS) or aflibercept (EYLEA) compared to anti-VEGF monotherapy in a swine model of laser induced choroidal neovascularization (CNV). Four animals per group, and six groups. The groups consist of (1) vehicle, (2) 300 μg, (3) 600 μg, (4) 900 μg, (5) aflibercept 2.0 mg, and (6) aflibercept 2.0 mg+ICON-1 (ICON-1.5 in parallel study) at 600 μg.

Pigs are lasered in both eyes (OU) on Day 0 (DO). Test articles and vehicle are dosed bilaterally via intravitreal (IVT) injection on D7. Ranibizumab (LUCENTIS) or aflibercept (EYLEA) are dosed on day 0 immediately after laser (DO). In the combination group (group 7), anti-VEGF agents are injected on DO and one-armed FVII-Fc immunoconjugates on D7.

Ocular Examination: Mydriasis for ocular examination is done using topical 1% tropicamide HCL (one drop in each eye 15 minutes prior to examination). Complete ocular examination (modified Hackett and McDonald) using a slit lamp biomicroscope and indirect ophthalmoscope to evaluate ocular surface morphology, anterior segment and posterior segment inflammation, cataract formation, and retinal changes are conducted by a veterinary ophthalmologist at baseline and D14.

Fluorescein angiography (FA): FA is done in both eyes of all animals on D7, D10, and D14 after laser. Mydriasis for FA is done using topical 1% Tropicamide HCL (one drop in each eye 15 minutes prior to examination). Full FA is performed for 1-5 minutes after intravenous sodium fluorescein injection (12 mg kg⁻¹). A reader analyzes the masked images obtained. The area of maximal fluorescein leakage is measured using Image J for each lesion.

All eyes are collected for ISH and flatmount analysis.

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

All patents, patent applications, patent application publications, journal articles and protocols referenced herein are incorporated by reference in their entireties, for all purposes.

1. An immunoconjugate comprising two dimerized immunoglobulin (Ig) Fc monomers, and a mutated factor VII protein, wherein the mutated factor VII protein is fused to only one of the Fc monomers, and wherein the mutated factor VII protein exhibits a decreased coagulation response in a mammalian host, as compared to a wild-type factor VII protein.

2. The immunoconjugate of claim 1, wherein the mutated factor VII protein exhibits no coagulation response in the mammalian host.

3. The immunoconjugate of claim 1 further comprising a linker sequence between the Ig Fc monomer and the factor VII protein.

4. The immunoconjugate of claim 1, wherein each of the Ig Fc monomers comprise a hinge sequence.

5. (canceled)

6. (canceled)

7. The immunoconjugate of claim 6, wherein the two dimerized Ig Fc monomers are linked together by one or more disulfide bonds.

8-16. (canceled)

17. The immunoconjugate of claim 1, wherein the mutated human FVII protein comprises a single point mutation at Lys341 or Ser344.

18. The immunoconjugate of claim 17, wherein the single point mutation is Lys341 to Ala341.

19-29. (canceled)

30. A composition comprising the immunoconjugate of claim 1.

31. A method for decreasing cancer-related neovascularization in a patient in need thereof, comprising administering to the patient the composition of claim 30.

32-36. (canceled)

37. A method for preventing, inhibiting, or reversing ocular neovascularization in an eye of a patient in need thereof, comprising administering to the patient the composition of claim 30.

38. (canceled)

39. The method of claim 37, wherein the neovascularization is associated with proliferative diabetic retinopathy, wet age-related macular degeneration (AMD), retinopathy of prematurity (ROP), or neovascular glaucoma.

40. (canceled)

41. The method of claim 37, wherein the neovascularization is choroidal neovascularization.

42-45. (canceled)

46. The method of claim 37, wherein administering comprises intravitreal injection or suprachoroidal injection.

47. (canceled)

48. (canceled)

49. The method of claim 37, wherein administering comprises multiple dosing sessions.

50-67. (canceled)

68. The method of claim 37, further comprises administering an effective amount of a neovascularization inhibitor to the patient.

69. (canceled)

70. (canceled)

71. The method of claim 68, wherein the neovascularization inhibitor is a vascular endothelial growth factor (VEGF) inhibitor, a VEGF receptor inhibitor, a platelet derived growth factor (PDGF) inhibitor or a PDGF receptor inhibitor.

72. The method of claim 68, wherein the neovascularization inhibitor is ranibizumab.

73-79. (canceled)

80. A formulation comprising the immunoconjugate of claim 1 and a pharmaceutically acceptable excipient.

81. The formulation of claim 80, wherein the formulation further comprises ranibizumab.

82-94. (canceled)

95. A composition comprising the immunoconjugate of claim 1, wherein the composition is substantially free of two-armed immunoconjugates.
